SEQUENCE LISTING (103 sequences)

SEQ ID NO: 1 - protein, 227 aa, synthetic construct
  REGION 1..227: note = Trastuzumab (anti-HER2) Fab HC
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 2 - protein, 214 aa, synthetic construct
  REGION 1..214: note = Trastuzumab (anti-HER2) Fab LC
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 247 aa, synthetic construct
  REGION 1..247: note = Trastuzumab (anti-HER2) scFv-LH
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGSEGKSSGSGSESKSTGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSS

SEQ ID NO: 4 - protein, 245 aa, synthetic construct
  REGION 1..245: note = Trastuzumab (anti-HER2) spFv-LH
DIQMTQSPSSLSASVGDRVTITCRASQDVNTAVAWYQQKPGCAPKLLIYSASFLYSGVPSRFSGSRSGTDFTLTISSLQPEDFATYYCQQHYTTPPTFGQGTKVEIKGGGSGGSGGCPPCGGSGGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGCGTLVTVSS

SEQ ID NO: 5 - protein, 5 aa, synthetic construct
  REGION 1..5: note = Trastuzumab HCDR1
DTYIH

SEQ ID NO: 6 - protein, 17 aa, synthetic construct
  REGION 1..17: note = Trastuzumab HCDR2
RIYPTNGYTRYADSVKG

SEQ ID NO: 7 - protein, 11 aa, synthetic construct
  REGION 1..11: note = Trastuzumab HCDR3
WGGDGFYAMDY

SEQ ID NO: 8 - protein, 11 aa, synthetic construct
  REGION 1..11: note = Trastuzumab LCDR1
RASQDVNTAVA

SEQ ID NO: 9 - protein, 7 aa, synthetic construct
  REGION 1..7: note = Trastuzumab LCDR2
SASFLYS

SEQ ID NO: 10 - protein, 9 aa, synthetic construct
  REGION 1..9: note = Trastuzumab LCDR3
QQHYTTPPT

SEQ ID NO: 11 - protein, 226 aa, synthetic construct
  REGION 1..226: note = Pertuzumab (anti-HER2) Fab HC
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 12 - protein, 214 aa, synthetic construct
  REGION 1..214: note = Pertuzumab (anti-HER2) Fab LC
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 5 aa, synthetic construct
  REGION 1..5: note = Pertuzumab HCDR1
DYTMD

SEQ ID NO: 14 - protein, 17 aa, synthetic construct
  REGION 1..17: note = Pertuzumab HCDR2
DVNPNSGGSIYNQRFKG

SEQ ID NO: 15 - protein, 10 aa, synthetic construct
  REGION 1..10: note = Pertuzumab HCDR3
NLGPSFYFDY

SEQ ID NO: 16 - protein, 11 aa, synthetic construct
  REGION 1..11: note = Pertuzumab LCDR1
KASQDVSIGVA

SEQ ID NO: 17 - protein, 7 aa, synthetic construct
  REGION 1..7: note = Pertuzumab LCDR2
SASYRYT

SEQ ID NO: 18 - protein, 9 aa, synthetic construct
  REGION 1..9: note = Pertuzumab LCDR3
QQYYIYPYT

SEQ ID NO: 19 - protein, 247 aa, synthetic construct
  REGION 1..247: note = C98hc 1
QVQLQESGPGLVKPSQTLSLTCTVSGGSISSGAYYWSWIRQHPGKGLEWIGYIYYSGSTYYNPSLKSRVTISLDTSKNQFSLKLSSVTAADTAVYYCARAGPKWELWFDYWGQGTLVTVSSGTEGKSSGSGSESKSTQSALTQPASVSGSPGQSITISCTGTSSDVDNYNYVSWYLQHPGKAPKLMIYDVSNRPSGISTRFSGSKSGNTASLTISGLQAEDEADYYCSSFTSSSTWVFGGGTKLTVL

SEQ ID NO: 20 - protein, 11 aa, synthetic construct
  REGION 1..11: note = HCDR1
GGSISSGAYYW

SEQ ID NO: 21 - protein, 7 aa, synthetic construct
  REGION 1..7: note = HCDR2
IYYSGST

SEQ ID NO: 22 - protein, 13 aa, synthetic construct
  REGION 1..13: note = HCDR3
ARAGPKWELWFDY

SEQ ID NO: 23 - protein, 9 aa, synthetic construct
  REGION 1..9: note = LCDR1
SSDVDNYNY

SEQ ID NO: 24 - protein, 7 aa, synthetic construct
  REGION 1..7: note = LCDR2
DVSNRPS

SEQ ID NO: 25 - protein, 10 aa, synthetic construct
  REGION 1..10: note = LCDR3
SSFTSSSTWV

SEQ ID NO: 26 - protein, 247 aa, synthetic construct
  REGION 1..247: note = CD98hc 2 scFv
QVQLVESGGGVVQPGRSLRLSCAASGFTFSSYGMHWVRQAPGKGLEWVAIISYDGSNKHYADSVKGRFTISRDNSKNTLYLQMNSLRAEDTAVYYCARAPSSFYFDYWGQGTLVTVSSGTEGKSSGSGSESKSTDIVMTQSPDSLAVSLGERATINCKSSQSVLFSSNNKNYLAWYQQKPGQPPKLLIYWASTRESGVPDRFSGSGSGTDFTLTISSLQAEDVAVYYCQQYYSTPPTFGQGTKVVIK

SEQ ID NO: 27 - protein, 8 aa, synthetic construct
  REGION 1..8: note = HCDR1
GFTFSSYG

SEQ ID NO: 28 - protein, 8 aa, synthetic construct
  REGION 1..8: note = HCDR2
ISYDGSNK

SEQ ID NO: 29 - protein, 11 aa, synthetic construct
  REGION 1..11: note = HCDR3
ARAPSSFYFDY

SEQ ID NO: 30 - protein, 12 aa, synthetic construct
  REGION 1..12: note = LCDR1
QSVLFSSNNKNY

SEQ ID NO: 31 - skipped

SEQ ID NO: 32 - protein, 9 aa, synthetic construct
  REGION 1..9: note = LCDR3
QQYYSTPPT

SEQ ID NO: 33 - protein, 248 aa, synthetic construct
  REGION 1..248: note = CD98hc 2 scFv- His cap
QVQLVESGGGVVQPGRSLRLSCAASGFTFSSYGMHWVRQAPGKGLEWVAIISYDGSNKHYADSVKGRFTISRDNSKNTLYLQMNSLRAEDTAVYYCARAPSSFYFDYWGQGTLVTVSSGTEGKSSGSGSESKSTDIVMTQSPDSLAVSLGERATINCKSSQSVLFSSNNKNYLAWYQQKPGQPPKLLIYWASTRESGVPDRFSGSGSGTDFTLTISSLQAEDVAVYYCQQYYSTPPTFGQGTKVVIKH

SEQ ID NO: 34 - protein, 249 aa, synthetic construct
  REGION 1..249: note = CD98hc 2 spFv
QVQLVESGGGVVQPGRSLRLSCAASGFTFSSYGMHWVRQAPGCGLEWVAIISYDGSNKHYADSVKGRFTISRDNSKNTLYLQMNSLRAEDTAVYYCARAPSSFYFDYWGQGTLVTVSSGGGSGGSGGCPPCGGSGGDIVMTQSPDSLAVSLGERATINCKSSQSVLFSSNNKNYLAWYQQKPGQPPKLLIYWASTRESGVPDRFSGSGSGTDFTLTISSLQAEDVAVYYCQQYYSTPPTFGCGTKVVIK

SEQ ID NO: 35 - protein, 250 aa, synthetic construct
  REGION 1..250: note = CD98hc 2 spFv- His cap
QVQLVESGGGVVQPGRSLRLSCAASGFTFSSYGMHWVRQAPGCGLEWVAIISYDGSNKHYADSVKGRFTISRDNSKNTLYLQMNSLRAEDTAVYYCARAPSSFYFDYWGQGTLVTVSSGGGSGGSGGCPPCGGSGGDIVMTQSPDSLAVSLGERATINCKSSQSVLFSSNNKNYLAWYQQKPGQPPKLLIYWASTRESGVPDRFSGSGSGTDFTLTISSLQAEDVAVYYCQQYYSTPPTFGCGTKVVIKH

SEQ ID NO: 36 - protein, 238 aa, synthetic construct
  REGION 1..238: note = CD98hc 3
QVQLQESGPGLVKPSETLSLTCTVSGGSISSSYWSWIRQPPGKGLEWIGYIYNSGSTNYNPSLKSRVTISVDTSKNQFSLKLSSVTAADTAVYYCARETYYFDYWGQGTLVTVSSGTEGKSSGSGSESKSTDIVMTQSQKFMSTSVGDRVSVTCKASQNVGINVAWYQKKPGQPPKALIYSAAYRYSGVPDRFTGSGSGTDFTLTISNVQSEDLAEYFCQQYNSYPLTFGSGTKLEIK

SEQ ID NO: 37 - protein, 8 aa, synthetic construct
  REGION 1..8: note = HCDR1
GGSISSSY

SEQ ID NO: 38 - protein, 7 aa, synthetic construct
  REGION 1..7: note = HCDR2
IYNSGST

SEQ ID NO: 39 - protein, 9 aa, synthetic construct
  REGION 1..9: note = HCDR3
ARETYYFDY

SEQ ID NO: 40 - protein, 6 aa, synthetic construct
  REGION 1..6: note = LCDR1
QNVGIN

SEQ ID NO: 41 - skipped

SEQ ID NO: 42 - protein, 9 aa, synthetic construct
  REGION 1..9: note = LCDR3
QQYNSYPLT

SEQ ID NO: 43 - protein, 251 aa, synthetic construct
  REGION 1..251: note = CD98hc 4
QVQLVQSGAEVKKPGASVKVSCKASGYTFTSYYIHWVRQAPGQGLEWMGLINPSGGTTNYPQKFQGRVTMTRDTSTSTVYMELSSLRSEDTAVYYCARDPNDYGGPKYFPHWGQGTLVTVSSGTEGKSSGSGSESKSTDIVMTQSPDSLAVSLGERATINCRSSQSVLYRSNNKNYLGWYQQKPGQPPKLLIYWASTRESGVPDRFSGSGSGTDFTLTISSLQAEDVAVYYCHQYYSTPLTFGGGTKVDIK

SEQ ID NO: 44 - protein, 8 aa, synthetic construct
  REGION 1..8: note = HCDR1
GYTFTSYY

SEQ ID NO: 45 - protein, 8 aa, synthetic construct
  REGION 1..8: note = HCDR2
INPSGGTT

SEQ ID NO: 46 - protein, 15 aa, synthetic construct
  REGION 1..15: note = HCDR3
ARDPNDYGGPKYFPH

SEQ ID NO: 47 - protein, 12 aa, synthetic construct
  REGION 1..12: note = LCDR1
QSVLYRSNNKNY

SEQ ID NO: 48 - protein, 9 aa, synthetic construct
  REGION 1..9: note = LCDR3
HQYYSTPLT

SEQ ID NO: 49 - protein, 253 aa, synthetic construct
  REGION 1..253: note = CD98hc5
QVQLVQSGAEVKKPGASVRVSCKASEYTFTSYYMHWIRQAPGQGLEWMGLINPSGGSTSYAQKFQGRVTMTRDTSTTTVYMELSSLRSEDTALYYCARAGYCTINSCFNWFDPWGQGTLVTVSSGTEGKSSGSGSESKSTDIVMTQSPDSLAESLGERATINCKSSQSVLYASRNKNYLAWYQQRPGQPPKLLIYWASTRESGVPDRFSGSGSGTDFTLTISSLQAEDVAIYYCQQYYSTPFTFGPGTKVDIK

SEQ ID NO: 50 - protein, 8 aa, synthetic construct
  REGION 1..8: note = HCDR1
EYTFTSYY

SEQ ID NO: 51 - protein, 8 aa, synthetic construct
  REGION 1..8: note = HCDR2
INPSGGST

SEQ ID NO: 52 - protein, 17 aa, synthetic construct
  REGION 1..17: note = HCDR3
ARAGYCTINSCFNWFDP

SEQ ID NO: 53 - protein, 12 aa, synthetic construct
  REGION 1..12: note = LCDR1
QSVLYASRNKNY

SEQ ID NO: 54 - protein, 8 aa, synthetic construct
  REGION 1..8: note = LCDR3
QYYSTPFT

SEQ ID NO: 55 - protein, 249 aa, synthetic construct
  REGION 1..249: note = CD98hc6
QVHLVQSGVEVKKPGASVKVSCKASGYTFTSYYIHWVRQAPGQGLEWMGLFNPSGGSTSYAQKFQGRVTLTRDTSTSTVYMELSSLRSEDTAVYYCARGGVGATGYFDYWGQGTLVTVSSGTEGKSSGSGSESKSTDIVMTQSPDSLAVSLGERATINCKSSQSVLYRSNNKNYLAWYQQKPGQPPKLLIYWASTRESGVPDRFSGSGSETDFTLTISSLQAEDVALYYCQQYYGTPFTFGPGTKVDIK

SEQ ID NO: 56 - protein, 8 aa, synthetic construct
  REGION 1..8: note = HCDR2
FNPSGGST

SEQ ID NO: 57 - protein, 13 aa, synthetic construct
  REGION 1..13: note = HCDR3
ARGGVGATGYFDY

SEQ ID NO: 58 - protein, 9 aa, synthetic construct
  REGION 1..9: note = LCDR3
QQYYGTPFT

SEQ ID NO: 59 - protein, 238 aa, synthetic construct
  REGION 1..238: note = CD98hc7
QVQLVQSGAEVKKPGASVKVSCKASGYTFTGYYLHWVRQAPGQGLEWMGWINPNSGGSNSAQKFQGRVTMTRDTSISTAYMEVSRLTSDDTAVYYCLRGKTFDPWGQGTLVTVSSGTEGKSSGSGSESKSTDIQLTQSPSFLSASVGDRVTITCRASQGISSHLAWYQQKPGKAPKLLIYAASTLQSGVPSRFSGSGSGTEFTLTISSLQPEDFATYYCQQLNNYPLTFGQGTRLEIK

SEQ ID NO: 60 - protein, 8 aa, synthetic construct
  REGION 1..8: note = HCDR1
GYTFTGYY

SEQ ID NO: 61 - protein, 8 aa, synthetic construct
  REGION 1..8: note = HCDR2
INPNSGGS

SEQ ID NO: 62 - protein, 8 aa, synthetic construct
  REGION 1..8: note = HCDR3
LRGKTFDP

SEQ ID NO: 63 - protein, 6 aa, synthetic construct
  REGION 1..6: note = LCDR1
QGISSH

SEQ ID NO: 64 - skipped

SEQ ID NO: 65 - protein, 9 aa, synthetic construct
  REGION 1..9: note = LCDR3
QQLNNYPLT

SEQ ID NO: 66 - protein, 253 aa, synthetic construct
  REGION 1..253: note = CD98hc8
QVQLVQSGAEVKKPGASVKVSCKASGYTFTSYYMHWVRQAPGQGLEWMGLINPSGGSTSYAQKFQGRVTMTRDTSTSTVYMELSSLRSEDTAVYYCARAGYCRSTTSCFNWFDPWGQGTLVTVSSGTEGKSSGSGSESKSTDIVMTQSPDSLAVSLGERATINCKSSQSVLYNSNNKNYLAWYQQKPGQPPKLLIYWASTRESGVPDRFSGSGSGTDFTLTFSSLQAEDVAVYYCQQYYSTPFTFGPGTKVDI

SEQ ID NO: 67 - protein, 7 aa, synthetic construct
  REGION 1..7: note = HCDR2
NPSGGST

SEQ ID NO: 68 - protein, 18 aa, synthetic construct
  REGION 1..18: note = HCDR3
ARAGYCRSTTSCFNWFDP

SEQ ID NO: 69 - protein, 12 aa, synthetic construct
  REGION 1..12: note = LCDR1
QSVLYNSNNKNY

SEQ ID NO: 70 - protein, 9 aa, synthetic construct
  REGION 1..9: note = LCDR3
QQYYSTPFT

SEQ ID NO: 71 - protein, 246 aa, synthetic construct
  REGION 1..246: note = CD98hc9
EVQLVESGGGLVQPGGSLRLSCAASGFTLSSSWMHWVRQAPGKGLVWVSRINSDGSSTSYADSVKGRFTISRDNAKNTLYLQMNSLRAEDTALYYCARWNWGWYFDLWGRGTLVTVSSGTEGKSSGSGSESKSTDIVMTQSPLSLPVTPGEPASISCRSSQSLLHSHGDNYLDWFLQKPGQSPQLLIYLGSNRASGVPDRFSGSGSGTDFTLKISRVEAEDVGVYYCMQTLETPYTFGQGTKLQIK

SEQ ID NO: 72 - protein, 8 aa, synthetic construct
  REGION 1..8: note = HCDR1
GFTLSSSW

SEQ ID NO: 73 - protein, 8 aa, synthetic construct
  REGION 1..8: note = HCDR2
INSDGSST

SEQ ID NO: 74 - protein, 11 aa, synthetic construct
  REGION 1..11: note = HCDR3
ARWNWGWYFDL

SEQ ID NO: 75 - protein, 11 aa, synthetic construct
  REGION 1..11: note = LCDR1
QSLLHSHGDNY

SEQ ID NO: 76 - skipped

SEQ ID NO: 77 - protein, 9 aa, synthetic construct
  REGION 1..9: note = LCDR3
MQTLETPYT

SEQ ID NO: 78 - protein, 247 aa, synthetic construct
  REGION 1..247: note = CD98hc10
EVQLVESGGGLVQPGGSLRLSCVASGFTFSSYWMHWVRQAPGKGLVWVSRINSDGSIIRYADSVKGRFTISRDNAKNTLYLQMNSLRAEDTAVYYCVREGRYTGSSLDYWGQGALVTVSSGTEGKSSGSGSESKSTQSVLTQPPSVSGAPGQRVAISCTGNNSNIGAGYDVHWYQQLPGTAPQLLIYGNSDRPSGVPDRFSGSKSGTSASLAITGLQAEDEAAYYCQSYDSSLTGWVFGGGTKLTVL

SEQ ID NO: 79 - protein, 8 aa, synthetic construct
  REGION 1..8: note = HCDR1
GFTFSSYW

SEQ ID NO: 80 - protein, 8 aa, synthetic construct
  REGION 1..8: note = HCDR2
INSDGSII

SEQ ID NO: 81 - protein, 13 aa, synthetic construct
  REGION 1..13: note = HCDR3
VREGRYTGSSLDY

SEQ ID NO: 82 - protein, 9 aa, synthetic construct
  REGION 1..9: note = LCDR1
NSNIGAGYD

SEQ ID NO: 83 - skipped

SEQ ID NO: 84 - protein, 11 aa, synthetic construct
  REGION 1..11: note = LCDR3
QSYDSSLTGWV

SEQ ID NO: 85 - protein, 247 aa, synthetic construct
  REGION 1..247: note = CD98hc11
QVQLVESGGGVVQPGRSLRLSCAASGFTFSSYGMHWVRQAPGKGLEWVAIISYDGSNKHYADSVKGRFTISRDNSKNTLYLQMNSLRAEDTAVYYCARAPSAFYFDYWGQGTLVTVSSGTEGKSSGSGSESKSTDIVMTQSPDSLAVSLGERATINCKSSQSVLFSSNNKNYLAWYQQKPGQPPKLLIYWASTRESGVPDRFSGSGSGTDFTLTISSLQAEDVAVYYCQQYYSTPPTFGQGTKVVIK

SEQ ID NO: 86 - protein, 11 aa, synthetic construct
  REGION 1..11: note = HCDR3
ARAPSAFYFDY

SEQ ID NO: 87 - protein, 248 aa, synthetic construct
  REGION 1..248: note = CD98hc11 His-cap
QVQLVESGGGVVQPGRSLRLSCAASGFTFSSYGMHWVRQAPGKGLEWVAIISYDGSNKHYADSVKGRFTISRDNSKNTLYLQMNSLRAEDTAVYYCARAPSAFYFDYWGQGTLVTVSSGTEGKSSGSGSESKSTDIVMTQSPDSLAVSLGERATINCKSSQSVLFSSNNKNYLAWYQQKPGQPPKLLIYWASTRESGVPDRFSGSGSGTDFTLTISSLQAEDVAVYYCQQYYSTPPTFGQGTKVVIKH

SEQ ID NO: 88 - protein, 249 aa, synthetic construct
  REGION 1..249: note = CD98hc11 spFv
QVQLVESGGGVVQPGRSLRLSCAASGFTFSSYGMHWVRQAPGCGLEWVAIISYDGSNKHYADSVKGRFTISRDNSKNTLYLQMNSLRAEDTAVYYCARAPSAFYFDYWGQGTLVTVSSGGGSGGSGGCPPCGGSGGDIVMTQSPDSLAVSLGERATINCKSSQSVLFSSNNKNYLAWYQQKPGQPPKLLIYWASTRESGVPDRFSGSGSGTDFTLTISSLQAEDVAVYYCQQYYSTPPTFGCGTKVVIK

SEQ ID NO: 89 - protein, 250 aa, synthetic construct
  REGION 1..250: note = CD98hc11 spFv His-cap
QVQLVESGGGVVQPGRSLRLSCAASGFTFSSYGMHWVRQAPGCGLEWVAIISYDGSNKHYADSVKGRFTISRDNSKNTLYLQMNSLRAEDTAVYYCARAPSAFYFDYWGQGTLVTVSSGGGSGGSGGCPPCGGSGGDIVMTQSPDSLAVSLGERATINCKSSQSVLFSSNNKNYLAWYQQKPGQPPKLLIYWASTRESGVPDRFSGSGSGTDFTLTISSLQAEDVAVYYCQQYYSTPPTFGCGTKVVIKH

SEQ ID NO: 90 - protein, 247 aa, synthetic construct
  REGION 1..247: note = CD98hc12
QVQLVESGGGVVQPGRSLRLSCAASGFTFSSYGMHWVRQAPGKGLEWVAIISYDGSNKHYADSVKGRFTISRDNSKNTLYLQMNSLRAEDTAVYYCARAPSHFYFDYWGQGTLVTVSSGTEGKSSGSGSESKSTDIVMTQSPDSLAVSLGERATINCKSSQSVLFSSNNKNYLAWYQQKPGQPPKLLIYWASTRESGVPDRFSGSGSGTDFTLTISSLQAEDVAVYYCQQYYSTPPTFGQGTKVVIK

SEQ ID NO: 91 - protein, 11 aa, synthetic construct
  REGION 1..11: note = HCDR3
ARAPSHFYFDY

SEQ ID NO: 92 - protein, 248 aa, synthetic construct
  REGION 1..248: note = CD98hc12His-cap
QVQLVESGGGVVQPGRSLRLSCAASGFTFSSYGMHWVRQAPGKGLEWVAIISYDGSNKHYADSVKGRFTISRDNSKNTLYLQMNSLRAEDTAVYYCARAPSHFYFDYWGQGTLVTVSSGTEGKSSGSGSESKSTDIVMTQSPDSLAVSLGERATINCKSSQSVLFSSNNKNYLAWYQQKPGQPPKLLIYWASTRESGVPDRFSGSGSGTDFTLTISSLQAEDVAVYYCQQYYSTPPTFGQGTKVVIKH

SEQ ID NO: 93 - protein, 249 aa, synthetic construct
  REGION 1..249: note = CD98hc12 spFv
QVQLVESGGGVVQPGRSLRLSCAASGFTFSSYGMHWVRQAPGCGLEWVAIISYDGSNKHYADSVKGRFTISRDNSKNTLYLQMNSLRAEDTAVYYCARAPSHFYFDYWGQGTLVTVSSGGGSGGSGGCPPCGGSGGDIVMTQSPDSLAVSLGERATINCKSSQSVLFSSNNKNYLAWYQQKPGQPPKLLIYWASTRESGVPDRFSGSGSGTDFTLTISSLQAEDVAVYYCQQYYSTPPTFGCGTKVVIK

SEQ ID NO: 94 - protein, 250 aa, synthetic construct
  REGION 1..250: note = CD98hc12 spFv His-cap
QVQLVESGGGVVQPGRSLRLSCAASGFTFSSYGMHWVRQAPGCGLEWVAIISYDGSNKHYADSVKGRFTISRDNSKNTLYLQMNSLRAEDTAVYYCARAPSHFYFDYWGQGTLVTVSSGGGSGGSGGCPPCGGSGGDIVMTQSPDSLAVSLGERATINCKSSQSVLFSSNNKNYLAWYQQKPGQPPKLLIYWASTRESGVPDRFSGSGSGTDFTLTISSLQAEDVAVYYCQQYYSTPPTFGCGTKVVIKH

SEQ ID NO: 95 - protein, 6 aa, synthetic construct
  REGION 1..6: note = Linker 1
GGSGGS

SEQ ID NO: 96 - protein, 20 aa, synthetic construct
  REGION 1..20: note = Linker 2
GGGGSGGGGSGGGGSGGGGS

SEQ ID NO: 97 - protein, 16 aa, synthetic construct
  REGION 1..16: note = Linker 3
GTEGKSSGSGSESKST

SEQ ID NO: 98 - protein, 20 aa, synthetic construct
  REGION 1..20: note = Linker 4
GGSEGKSSGSGSESKSTGGS

SEQ ID NO: 99 - protein, 18 aa, synthetic construct
  REGION 1..18: note = Linker 5
GGGSGGSGGCPPCGGSGG

SEQ ID NO: 100 - protein, 719 aa, synthetic construct
  REGION 1..719: note = BBBB1819 HC1
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGKGGGGSGGGGSGGGGSGGGGSQVQLVESGGGVVQPGRSLRLSCAASGFTFSSYGMHWVRQAPGCGLEWVAIISYDGSNKHYADSVKGRFTISRDNSKNTLYLQMNSLRAEDTAVYYCARAPSAFYFDYWGQGTLVTVSSGGGSGGSGGCPPCGGSGGDIVMTQSPDSLAVSLGERATINCKSSQSVLFSSNNKNYLAWYQQKPGQPPKLLIYWASTRESGVPDRFSGSGSGTDFTLTISSLQAEDVAVYYCQQYYSTPPTFGCGTKVVIKH

SEQ ID NO: 101 - protein, 477 aa, synthetic construct
  REGION 1..477: note = BBBB1819 HC2
DIQMTQSPSSLSASVGDRVTITCRASQDVNTAVAWYQQKPGCAPKLLIYSASFLYSGVPSRFSGSRSGTDFTLTISSLQPEDFATYYCQQHYTTPPTFGQGTKVEIKGGGSGGSGGCPPCGGSGGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGCGTLVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 102 - protein, 719 aa, synthetic construct
  REGION 1..719: note = BBBB1809 HC1
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGKGGGGSGGGGSGGGGSGGGGSQVQLVESGGGVVQPGRSLRLSCAASGFTFSSYGMHWVRQAPGCGLEWVAIISYDGSNKHYADSVKGRFTISRDNSKNTLYLQMNSLRAEDTAVYYCARAPSAFYFDYWGQGTLVTVSSGGGSGGSGGCPPCGGSGGDIVMTQSPDSLAVSLGERATINCKSSQSVLFSSNNKNYLAWYQQKPGQPPKLLIYWASTRESGVPDRFSGSGSGTDFTLTISSLQAEDVAVYYCQQYYSTPPTFGCGTKVVIKH

SEQ ID NO: 103 - protein, 477 aa, synthetic construct
  REGION 1..477: note = BBBB1809 HC2
DIQMTQSPSSLSASVGDRVTITCRASQDVNTAVAWYQQKPGCAPKLLIYSASFLYSGVPSRFSGSRSGTDFTLTISSLQPEDFATYYCQQHYTTPPTFGQGTKVEIKGGGSGGSGGCPPCGGSGGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGCGTLVTVSSEPKSSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2026/0337295 A1
(43) Pub. Date: Oct. 29, 2026

(54) CD98 BINDING CONSTRUCTS FOR TREATING BRAIN TUMORS

(71) Applicant: Janssen Biotech, Inc., Horsham, PA (US)

(72) Inventors: Wan Cheung CHEUNG, Raritan, NJ (US); Suzanne EDAVETTAL, Horsham, PA (US); James PATTERSON, Raritan, NJ (US); Matthew BUSCHMAN, Raritan, NJ (US); Justin SCHEER, Raritan, NJ (US); Fern SHA, Raritan, NJ (US); Isha TANEJA, Raritan, NJ (US)

(21) Appl. No.: 19/101,325

(22) PCT Filed: Jul. 28, 2023
(86) PCT No.: PCT/IB2023/057723

§ 371 Date: Feb. 5, 2025

Related U.S. Application Data
(60) Provisional application No. 63/395,389, filed on Aug. 5, 2022.

(51) Int. Cl. C07K 16/32; A61K 39/00; A61P 35/00; C07K 16/28

(52) U.S. Cl. CPC C07K 16/32; A61P 35/00; C07K 16/2896; A61K 2039/505; C07K 2317/31; C07K 2317/35; C07K 2317/52; C07K 2317/55; C07K 2317/622; C07K 2317/732

(57) ABSTRACT

Provided are novel multispecific antibodies comprising at least one of a first antigen-binding region and a second antigen-binding region each capable of binding specifically to human epidermal growth factor receptor 2 (HER2), and a third antigen-binding region capable of binding specifically to CD98.

### CROSS REFERENCE TO RELATED APPLICATION

This application claims the benefit of U.S. Provisional Patent Application No. 63/395,389, filed Aug. 5, 2022, the disclosure of which is incorporated by reference herein in entirety.

### REFERENCE TO SEQUENCE LISTING SUBMITTED ELECTRONICALLY

This application contains a sequence listing, which is submitted electronically. The content of the electronic sequence listing (JBI6744WOPCT1 Sequence Listing.xml; size: 131,642 bytes; and date of creation: Jul. 11, 2023) is herein incorporated by reference in its entirety.

### TECHNICAL FIELD

The present disclosure relates to novel multispecific antibodies comprising a first means capable of binding to a first antigen; and a second means capable of binding to a second antigen on a particular tissue, e.g., on a blood brain barrier.

### BACKGROUND

Numerous approaches have been studied to improve the delivery of therapeutic monoclonal antibodies (mAbs). For example, while the blood-brain barrier (BBB) prevents harmful substances from entering the brain and is essential for brain homeostasis, it presents a formidable obstacle for efficiently delivering drugs to the brain. Large molecules, such as monoclonal antibodies and other biotherapeutics, have great therapeutic/diagnostic potential for treating/detecting pathology in the central nervous system (CNS). However, their route into the brain is prevented by the BBB. Numerous approaches have been studied to improve the brain delivery of therapeutic monoclonal antibodies (mAbs), including the use of receptor-mediated transcytosis (RMT). RMT utilizes abundantly expressed receptors on the luminal side of the BBB for transport through brain endothelial cells. Previous efforts to generate a clinically feasible platform for delivery of therapeutic mAbs into the brain have been focused on antibody engineering to increase the efficiency of transcytosis, with gains made through observations on valency of binding, pH dependency and affinity (reviewed in Goulatis et al., 2017, Curr Opin Struct Biol 45:109-115). However, translation into non-human primates (NHPs) and the clinic has been limited by rapid peripheral clearance from target-mediated drug disposition (TMDD) and safety from acute reticulocyte depletion (Gadkar K, et al. Eur J Pharm Biopharm. 2016; 101:53-61).

CD98 heavy chain subunit (CD98 hc) is a member of the solute carrier family and heterodimerizes with a number of CD98 light chain members to form amino acid transporters at the BBB (Zuchero Y J, et al. Neuron. 2016; 89(1): 70-82, citing Boado R J, et al. PNAS 1999; 96(21): 12079-84). The intracellular portion of CD98 hc functions to mediate integrin signaling, which plays a role in both cell growth and tumorigenesis (Zuchero Y J, et al. Neuron. 2016; 89(1): 70-82, citing Feral C C, et al. J Cell Biol 2007; 178: 701-711; Cantor J M and Ginsburg M H. J Cell Sci 2012; 125: 1373-82). CD98 hc is highly expressed on human brain microvasculature, and anti-CD98 hc bispecific antibodies have been used to deliver a therapeutic antibody to the mouse brain (Zuchero Y J, et al. Neuron. 2016; 89(1): 70-82).

Therefore, there is a need for a platform that can be used to shuttle drugs into the target tissue efficiently with improved safety and pharmacokinetics.

### BRIEF SUMMARY

In one aspect, provided herein is a multispecific antibody or antigen binding fragment thereof comprising at least one of a first antigen-binding region and a second antigen-binding region each capable of binding specifically to human epidermal growth factor receptor 2 (HER2), and a third antigen-binding region capable of binding specifically to CD98.

In certain embodiments, the first antigen-binding region comprises a first heavy chain variable region (VH1) comprising a heavy chain complementarity determining region 1 (HCDR1), a HCDR2, and a HCDR3 comprising the amino acid sequences of SEQ ID NOs: 5, 6 and 7, respectively; and a first light chain variable region (VL1) comprising a light chain complementarity determining region 1 (LCDR1), a LCDR2, and a LCDR3 comprising the amino acid sequences of SEQ ID NOs: 8, 9 and 10, respectively; the second antigen-binding region comprises second heavy chain variable region (VH2) comprising a heavy chain complementarity determining region 1 (HCDR1), a HCDR2, and a HCDR3 comprising the amino acid sequences of SEQ ID NOs: 13, 14 and 15, respectively; and a second light chain variable region (VL2) comprising a light chain complementarity determining region 1 (LCDR1), a LCDR2, and a LCDR3 comprising the amino acid sequences of SEQ ID NOs: 16, 17 and 18, respectively; and the third antigen-binding region comprises a first single chain variable fragment (scFv1) having: a third heavy chain variable region (VH3) comprising a heavy chain complementarity determining region 1 (HCDR1), a HCDR2, and a HCDR3, and a third light chain variable region (VL3) comprising a light chain complementarity determining region 1 (LCDR1), a LCDR2, and a LCDR3, wherein the HCDR1, HCDR2, HCDR3, LCDR1, LCDR2 and LCDR3 have any of the amino acid sequences of Table 2.

In certain embodiments, the VH1 an amino acid sequence identical to the VH of the HC sequence identified as SEQ ID NO: 1; and the VL1 comprises an amino acid sequence identical to the VL of the LC sequence identified as SEQ ID NO: 2; the VH2 an amino acid sequence identical to the VH of the HC sequence identified as SEQ ID NO: 11; and the VL2 comprises an amino acid sequence identical to the VL of the LC sequence identified as SEQ ID NO: 12; and the VH3 and the VL3 comprise amino acid sequences are identical to the respective VH and VL of a scFv identified as:
(i) SEQ ID NO: 19;
(ii) SEQ ID NO: 26;
(iii) SEQ ID NO: 33;
(iv) SEQ ID NO: 34;
(v) SEQ ID NO: 35;
(vi) SEQ ID NO: 36;
(vii) SEQ ID NO: 43;
(viii) SEQ ID NO: 49;
(ix) SEQ ID NO: 55;
(x) SEQ ID NO: 59;
(xi) SEQ ID NO: 66;
(xii) SEQ ID NO: 71;
(xiii) SEQ ID NO: 78;
(xiv) SEQ ID NO: 85;
(XV) SEQ ID NO: 87;
(xvi) SEQ ID NO: 88;
(xvii) SEQ ID NO: 89;
(xviii) SEQ ID NO: 90;
(xix) SEQ ID NO: 92;
(xx) SEQ ID NO: 93;
(xxi) SEQ ID NO: 94.

In certain embodiments, the multispecific antibody or antigen binding fragment thereof comprises (a) a first heavy chain (HC1) comprising the VH1, a first heavy chain constant region comprising a first Fc region (Fc1), and the scFv1, and (b) a first light chain (LC1) comprising the VL1 and a light chain constant region.

In certain embodiments, the multispecific antibody or antigen binding fragment thereof comprises (a) a second heavy chain (HC2) comprising the VH1 and a first heavy chain constant region comprising a second Fc region (Fc2), and (b) a second light chain (LC2) comprising the VL1 and a light chain constant region.

In certain embodiments, the multispecific antibody or antigen binding fragment thereof further comprises a second Fc region (Fc2).

In certain embodiments, the multispecific antibody or antigen binding fragment thereof comprises (a) a first heavy chain (HC1) comprising the VH2, and a first heavy chain constant region comprising a first Fc region (Fc1), (b) a first light chain (LC1) comprising the VL2 and a light chain constant region; and (c) a second heavy chain (HC2) comprising the scFv1, and a second heavy chain constant region comprising a second Fc region (Fc2).

In certain embodiments, the multispecific antibody or antigen binding fragment thereof comprises the first antigen binding region, the second antigen binding region and the third antigen binding region.

In certain embodiments, the multispecific antibody or antigen binding fragment thereof comprises (a) a first heavy chain (HC1) comprising the VH1, a first heavy chain constant region comprising a first Fc region (Fc1), and the scFv1, (b) a first light chain (LC1) comprising the VL1 and a light chain constant region; and (c) a second heavy chain (HC2) comprising a second single chain variable fragment (scFv2) and a first heavy chain constant region comprising a second Fc region (Fc2), wherein the scFv2 comprises the VH2 and VL2.

In certain embodiments, the multispecific antibody or antigen binding fragment thereof comprises (a) a first heavy chain (HC1) comprising the VH2, a first heavy chain constant region comprising a first Fc region (Fc1), and the scFv1, (b) a first light chain (LC1) comprising the VL2 and a light chain constant region; and (c) a second heavy chain (HC2) comprising a second single chain variable fragment (scFv2) and a first heavy chain constant region comprising a second Fc region (Fc2), wherein the scFv2 comprises the VH1 and VL1.

In certain embodiments, the scFv1 and/or scFv2 comprises at least one of (a) a first disulfide bond between a structurally conserved surface exposed VH cysteine (Cys) and a first L Cys; and b) a second disulfide bond between a structurally conserved surface exposed VL Cys and a second L Cys.

In certain embodiments, the scFv1 and scFv2 each independently comprise the first disulfide bond and the second disulfide bond.

In certain embodiments, the scFv2 comprises an amino acid sequence having an amino acid sequence of SEQ ID NO: 3 or 4.

In certain embodiments, the scFv1 comprises an amino acid sequence selected from the group consisting of: SEQ ID NO: 19, SEQ ID NO: 26, SEQ ID NO: 33, SEQ ID NO: 34, SEQ ID NO: 35, SEQ ID NO: 36, SEQ ID NO: 43, SEQ ID NO: 49, SEQ ID NO: 55, SEQ ID NO: 59, SEQ ID NO: 66, SEQ ID NO: 71, SEQ ID NO: 78, SEQ ID NO: 85, SEQ ID NO: 87, SEQ ID NO: 88, SEQ ID NO: 89, SEQ ID NO: 90, SEQ ID NO: 92, SEQ ID NO: 93, SEQ ID NO: 94.

In certain embodiments, each of the Fc1 and Fc2 comprises one or more heterodimeric mutations, or one or more knob and hole mutations.

In certain embodiments, the heterodimeric mutations comprise amino acid modifications at positions T350, L351, F405, and Y407 in one of Fc1 and Fc2, and amino acid modifications at positions T350, T366, K392 and T394 in the other one of Fc1 and Fc2, wherein the amino acid modification at position T350 is T350V, T350I, T350L or T350M; the amino acid modification at position L351 is L351Y; the amino acid modification at position F405 is F405A, F405V, F405T or F405S; the amino acid modification at position Y407 is Y407V, Y407A or Y407I; the amino acid modification at position T366 is T366L, T366I, T366V or T366M, the amino acid modification at position K392 is K392F, K392L or K392M, and the amino acid modification at position T394 is T394W, and wherein the numbering of amino acid residues is according to the EU index as set forth in Kabat.

In certain embodiments, one of Fc1 and Fc2 comprises mutations T350V, L351Y, F405A and Y407V, and the other of Fc1 and Fc2 comprises mutations T350V, T366L, K392L and T394W.

In certain embodiments, each of the Fc1 and Fc2 comprises one or more knob and hole mutations.

In certain embodiments, the multispecific antibody or antigen binding fragment thereof comprises a Fc domain having amino acid modifications that enhance binding of the multispecific antibody or antigen binding fragment thereof to a neonatal Fc receptor (RcRn), preferably the amino acid modifications enhance the binding at an acidic pH, more preferably the Fc domain has the M252Y/S254T/T256E (YTE) mutations, wherein the numbering of amino acid residues is according to the EU index as set forth in Kabat.

In certain embodiments, the multispecific antibody or antigen binding fragment thereof comprises a Fc domain having amino acid modifications that reduce or eliminate the effector function, preferably the Fc domain has one or more amino acid modifications at positions L234, L235, D265, D270, N297, E318, K320, K322, P331, and P329, such as one, two, three or four amino acid modifications of L234A, L235A, D265S and P331S, wherein the numbering of amino acid residues is according to the EU index as set forth in Kabat.

In certain embodiments, the multispecific antibody or antigen binding fragment thereof comprises a Fc domain having one or more amino acid modifications of M252Y, S254T, T256E, L234A, L235A and D265S, wherein the numbering of amino acid residues is according to the EU index as set forth in Kabat.

In certain embodiments, the multispecific antibody or antigen binding fragment thereof comprises a Fc domain not having amino acid modifications that reduce or eliminate the effector function.

Also provided is a multispecific antibody comprising a first heavy chain, a light chain and a second heavy chain each having the amino acid sequences at least 90% identical to (a) SEQ ID NO: 100, SEQ ID NO: 12, and SEQ ID NO: 101, respectively; or (b) SEQ ID NO: 102, SEQ ID NO: 12, and SEQ ID NO: 103, respectively;

wherein the first antigen-binding region is capable of binding specifically to a first epitope of HER2, the second antigen-binding region is capable of binding specifically to a second epitope of HER2, and the third antigen-binding region is capable of binding specifically to CD98.

In certain embodiments, the first heavy chain, the light chain and the second heavy chain each comprises the amino acid sequences of (a) SEQ ID NO: 100, SEQ ID NO: 12, and SEQ ID NO: 101, respectively; or (b) SEQ ID NO: 213, SEQ ID NO: 12, and SEQ ID NO: 103, respectively.

Another general aspect of the application relates to an isolated nucleic acid sequence encoding the multispecific antibody or antigen binding fragment thereof according to the application. Also provided is a vector comprising the isolated nucleic acid of the application, and a host cell comprising the isolated nucleic acids or vectors of the application.

Another general aspect of the application relates to a method of producing a multispecific antibody or antigen binding fragment thereof. The method comprises culturing a cell comprising a nucleic acid of the application under conditions to produce the multispecific antibody or antigen binding fragment thereof and recovering the multispecific antibody or antigen binding fragment thereof.

Further provided is a pharmaceutical composition comprising the multispecific antibody or antigen binding fragment thereof of the application and a pharmaceutically acceptable carrier.

Yet another general aspect of the invention relates to a method of treating or detecting a disorder, preferably a cancer, in a subject in need thereof, comprising administering to the subject the multispecific antibody or antigen-binding fragment of or the pharmaceutical composition of the application.

Other aspects, features and advantages of the invention will be apparent from the following disclosure, including the detailed description of the invention and its preferred embodiments and the appended claims.

### BRIEF DESCRIPTION OF THE FIGURES

FIG. 1 shows a representation of a biparatopic, multi-specific antibody fused with tissue targeting module.

FIG. 2 illustrates the molecular architecture of the TEM mAbs where the single CD98 scFv or CD98 spFv was fused to the C-terminus of one heavy chain of a biparatopic therapeutic mAb targeting HER2 by using a short, flexible linker.

FIGS. 3A-E show binding of biparatopic antibodies to (A) BT474, (B) MBA-MB-361, (C) JIMT-1, (D) U87-MG and (E) HCC1954 cells. Binding was compared to isotype control (CNTO3930) and silent trastuzumab and silent pertuzumab.

FIGS. 4A-D show curves of the spheroid area formed by (A, C) BT474 and (B, D) MDA-MB-361 cells cocultured at 1:1 E:T with iMG, in presence of anti HER2 TEM mAbs, trastuzumab, or isotype IgG1 at increasing doses.

FIGS. 5A-C show curves of red fluorescence for (A) JIMT-1, (B) MDA-MB-361, and (C) BT474 cells co-cultured at 10:1 E:T with human PBMCs in presence of anti HER2 TEM mAbs, trastuzumab, or isotype IgG1 at increasing doses.

FIG. 6 shows the PK of TEM mAbs in non-human primates. The TEM and control IgG1 mAbs were administered into cynomolgus monkeys at 10 mg/kg via slow bolus IV injection. Blood for PK was collected at 1, 6, 24, 72 and 168 h post dosing and processed to serum via the testing facility lab protocol. Following terminal blood collection, animals were euthanized at 72 and 168 h (n=2 at each time point). Approximately 200 mg of tissue was isolated from predefined brain locations (frontal lobe, hippocampus and temporal lobe).

FIGS. 7A-B show antitumor activity of BBBB1819 on SC BT474 human HER2-amplified breast cancer xenografts in mice (SC BT474 mice tumor xenograft model). FIG. 7A shows mean tumor volume in mice treated with vehicle or varying doses of BBBB1819 (CD98×HER2) antibody administered on Days 12, 15, 18, 21, 24, 27, 30, and 33 (indicated by black line beneath the X-axis). FIG. 7B shows mean tumor volume in mice treated with vehicle, BBB1819, ERBB2077 (the equivalent antibody format to BBBB1819, but lacking the CD98 spFv), or trastuzumab at 20 mg/kg on Days 12, 19, 26, and 33 (indicated by black line beneath the X-axis). Group tumor volumes are plotted as mean±SEM of n=10 animals/group. Data graphically represented when at least two thirds the number of mice remained in the study.

FIGS. 8A-D show pharmacokinetic (PK) analyses in the SC BT474 mice tumor xenograft model. FIG. 8A shows plasma concentration of varying doses of BBB1819 antibody over time. FIG. 8B shows plasma concentration of BBBB1819, ERBB2077 and trastuzumab antibodies over time. FIG. 8C shows tumor concentration of BBBB1819, ERBB2077 and trastuzumab antibodies over time. FIG. 8D shows tumor to plasma (%) ratio of BBBB1819, ERBB2077 and trastuzumab antibodies over time.

FIGS. 9A-C show in vivo biodistribution of Zr89-DFO*-HER2×CD98 (HER2×CD98) antibody in C57BL6 (B6) and human CD98 knock-in (CD98) mice. FIG. 9A shows standard uptake value (SUV) of the Zr89-DFO*-HER2×CD98 antibody in tissues on day 1. FIG. 9B shows SUV of the Zr89-DFO*-HER2×CD98 antibody in tissues on day 5. FIG. 9C shows SUV of the Zr89-DFO*-HER2×CD98 antibody in tissues on day 7.

FIGS. 10A-D show brain and heart uptake of HER2×CD98 and Zr89-DFO*-HER2 (HER2) antibodies in C57BL6 (BL6) and human CD98 knock-in (huCD98 KI) mice. FIG. 10A shows SUV in the brain on day 1. FIG. 10B shows SUV in the brain on day 5. FIG. 10C shows brain to heart ratio of SUV on day 1. FIG. 10D shows brain to heart ratio of SUV on day 5.

FIGS. 11A-C show brain, heart and total body uptake of HER2×CD98 antibodies in C57BL6 (BL6) and human CD98 knock-in (huCD98 KI) mice. FIG. 11A shows SUV in the brain post-injection. FIG. 11B shows SUV in the heart post-injection. FIG. 11C shows the megabecquerel (MBq) measurement in the total body of mice post-injection.

FIGS. 12A-12C show pharmacokinetics (PK) of BBB1819, ERBB2077, and trastuzumab (ERBB218) antibodies in human CD98 knock-in (CD98) mice at 24 hours post-injection. FIG. 12A shows BBB1819, ERBB2077, and ERBB128 antibodies concentration in the brain. FIG. 12B shows BBB1819, ERBB2077, and ERBB128 antibodies concentration in the plasma. FIG. 12C shows brain to plasma (%) ration of BBB1819, ERBB2077, and ERBB128 antibodies.

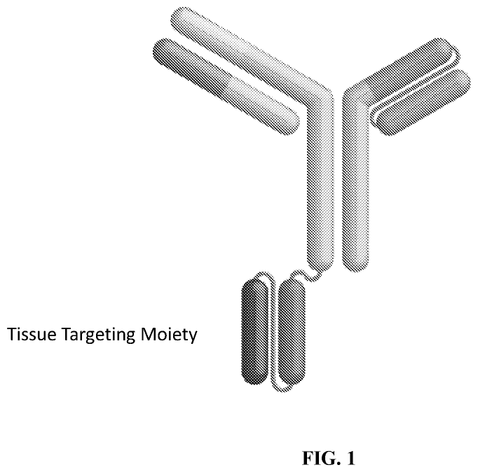

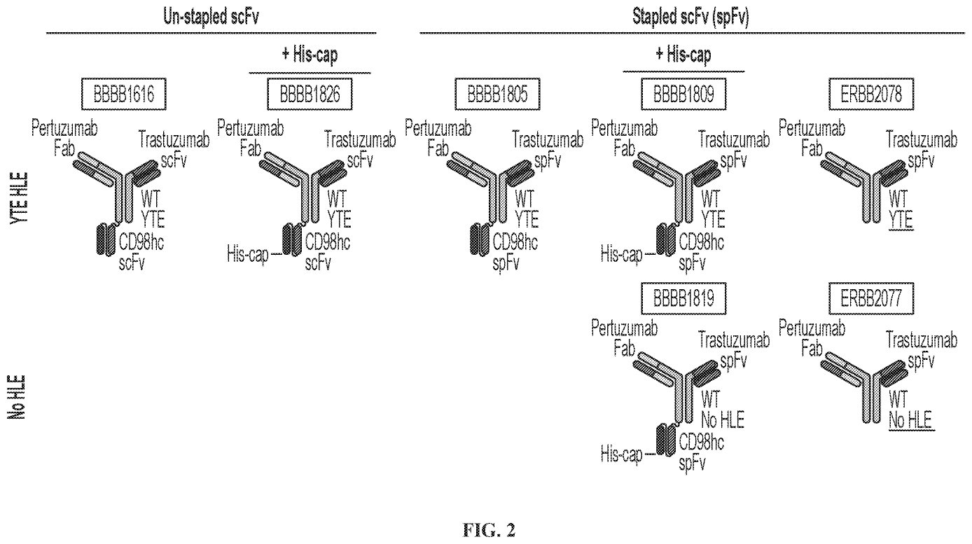

### DETAILED DESCRIPTION

The present invention relates to multispecific antibodies comprising at least one of a first antigen-binding region and a second antigen-binding region each capable of binding specifically to human epidermal growth factor receptor 2 (HER2), and a third antigen-binding region capable of binding specifically to CD98.

### Definitions

Techniques and procedures described or referenced herein include those that are generally well understood and/or commonly employed using conventional methodology by those skilled in the art, such as, for example, the widely utilized methodologies described in Sambrook et al., Molecular Cloning: A Laboratory Manual (3d ed. 2001); Current Protocols in Molecular Biology (Ausubel et al. eds., 2003); Therapeutic Monoclonal Antibodies: From Bench to Clinic (An ed. 2009); Monoclonal Antibodies: Methods and Protocols (Albitar ed. 2010); and Antibody Engineering Vols 1 and 2 (Kontermann and Dübel eds., 2d ed. 2010). Unless otherwise defined herein, technical and scientific terms used in the present description have the meanings that are commonly understood by those of ordinary skill in the art. For purposes of interpreting this specification, the following description of terms will apply and whenever appropriate, terms used in the singular will also include the plural and vice versa. In the event that any description of a term set forth conflicts with any document incorporated herein by reference, the description of the term set forth below shall control.

The term “antibody,” “immunoglobulin,” or “Ig” is used interchangeably herein, and is used in the broadest sense and specifically covers, for example, monoclonal antibodies (including agonist, antagonist, neutralizing antibodies, full length or intact monoclonal antibodies), antibody compositions with polyepitopic or monoepitopic specificity, polyclonal or monovalent antibodies, multivalent antibodies, multispecific antibodies (e.g., bispecific antibodies so long as they exhibit the desired biological activity), formed from at least two intact antibodies, single chain antibodies, and fragments thereof (e.g., domain antibodies), as described below. An antibody can be human, humanized, chimeric and/or affinity matured, as well as an antibody from other species, for example, mouse, rabbit, llama, etc. The term “antibody” is intended to include a polypeptide product of B cells within the immunoglobulin class of polypeptides that is able to bind to a specific molecular antigen and is composed of two identical pairs of polypeptide chains, wherein each pair has one heavy chain (about 50-70 kDa) and one light chain (about 25 kDa), each amino-terminal portion of each chain includes a variable region of about 100 to about 130 or more amino acids, and each carboxy-terminal portion of each chain includes a constant region. See, e.g., Antibody Engineering (Borrebaeck ed., 2d ed. 1995); and Kuby, Immunology (3d ed. 1997). Antibodies also include, but are not limited to, synthetic antibodies, recombinantly produced antibodies, antibodies including from Camelidae species (e.g., llama or alpaca) or their humanized variants, intrabodies, anti-idiotypic (anti-Id) antibodies, and functional fragments (e.g., antigen binding fragments) of any of the above, which refers to a portion of an antibody heavy or light chain polypeptide that retains some or all of the binding activity of the antibody from which the fragment was derived. Non-limiting examples of functional fragments (e.g., antigen binding fragments) include single-chain Fvs (scFv) (e.g., including monospecific, bispecific, etc.), Fab fragments, F(ab′) fragments, F(ab)₂ fragments, F(ab′)₂ fragments, disulfide-linked Fvs (dsFv), Fd fragments, Fv fragments, diabody, triabody, tetrabody, and minibody. In particular, antibodies provided herein include immunoglobulin molecules and immunologically active portions of immunoglobulin molecules, for example, antigen-binding domains or molecules that contain an antigen-binding site that binds to an antigen (e.g., one or more CDRs of an antibody). Such antibody fragments can be found in, for example, Harlow and Lane, Antibodies: A Laboratory Manual (1989); Mol. Biology and Biotechnology: A Comprehensive Desk Reference (Myers ed., 1995); Huston et al., 1993, Cell Biophysics 22:189-224; Plückthun and Skerra, 1989, Meth. Enzymol. 178:497-515; and Day, Advanced Immunochemistry (2d ed. 1990). The antibodies provided herein can be of any class (e.g., IgG, IgE, IgM, IgD, and IgA) or any subclass (e.g., IgG1, IgG2, IgG3, IgG4, IgA1, and IgA2) of immunoglobulin molecule. Antibodies may be agonistic antibodies or antagonistic antibodies. Antibodies may be neither agonistic nor antagonistic.

An “antigen” is a structure to which an antibody can selectively bind. A target antigen may be a polypeptide, carbohydrate, nucleic acid, lipid, hapten, or other naturally occurring or synthetic compound. In some embodiments, the target antigen is a polypeptide. In certain embodiments, an antigen is associated with a cell, for example, is present on or in a cell.

An “intact” antibody is one comprising an antigen-binding site as well as a CL and at least heavy chain constant regions, CH1, CH2 and CH3. The constant regions may include human constant regions or amino acid sequence variants thereof. In certain embodiments, an intact antibody has one or more effector functions.

The terms “binds” or “binding” refer to an interaction between molecules including, for example, to form a complex. Interactions can be, for example, non-covalent interactions including hydrogen bonds, ionic bonds, hydrophobic interactions, and/or van der Waals interactions. A complex can also include the binding of two or more molecules held together by covalent or non-covalent bonds, interactions, or forces. The strength of the total non-covalent interactions between a single antigen-binding site on an antibody and a single epitope of a target molecule, such as an antigen, is the affinity of the antibody or functional fragment for that epitope. The ratio of dissociation rate (k_{off}) to association rate (kₒₙ) of a binding molecule (e.g., an antibody) to a monovalent antigen (k_{off}/kₒₙ) is the dissociation constant K_{D}, which is inversely related to affinity. The lower the K_{D} value, the higher the affinity of the antibody. The value of K_{D} varies for different complexes of antibody and antigen and depends on both kₒₙ and k_{off}. The dissociation constant K_{D} for an antibody provided herein can be determined using any method provided herein or any other method well known to those skilled in the art. The affinity at one binding site does not always reflect the true strength of the interaction between an antibody and an antigen. When complex antigens containing multiple, repeating antigenic determinants, such as a polyvalent antigen, come in contact with antibodies containing multiple binding sites, the interaction of antibody with antigen at one site will increase the probability of a reaction at a second site. The strength of such multiple interactions between a multivalent antibody and antigen is called the avidity.

In connection with the binding molecules described herein terms such as “bind to,” “that specifically bind to,” and analogous terms are also used interchangeably herein and refer to binding molecules of antigen binding domains that specifically bind to an antigen, such as a polypeptide. A binding molecule or antigen binding domain that binds to or specifically binds to an antigen can be identified, for example, by immunoassays, Octet®, Biacore®, or other techniques known to those of skill in the art. In some embodiments, a binding molecule or antigen binding domain binds to or specifically binds to an antigen when it binds to an antigen with higher affinity than to any cross-reactive antigen as determined using experimental techniques, such as radioimmunoassay (RIA) and enzyme linked immunosorbent assay (ELISA). Typically, a specific or selective reaction will be at least twice background signal or noise and may be more than 10 times background. See, e.g., Fundamental Immunology 332-36 (Paul ed., 2d ed. 1989) for a discussion regarding binding specificity. In certain embodiments, the extent of binding of a binding molecule or antigen binding domain to a “non-target” protein is less than about 10% of the binding of the binding molecule or antigen binding domain to its particular target antigen, for example, as determined by fluorescence activated cell sorting (FACS) analysis or RIA. A binding molecule or antigen binding domain that binds to an antigen includes one that is capable of binding the antigen with sufficient affinity such that the binding molecule is useful, for example, as a therapeutic and/or diagnostic agent in targeting the antigen. In certain embodiments, a binding molecule or antigen binding domain that binds to an antigen has a dissociation constant (K_{D}) of less than or equal to 1 μM, 800 nM, 600 nM, 550 nM, 500 nM, 300 nM, 250 nM, 100 nM, 50 nM, 10 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, or 0.1 nM. In certain embodiments, a binding molecule or antigen binding domain binds to an epitope of an antigen that is conserved among the antigen from different species.

In certain embodiments, the binding molecules or antigen binding domains can comprise “chimeric” sequences in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; and Morrison et al., 1984, Proc. Natl. Acad. Sci. USA 81:6851-55). Chimeric sequences may include humanized sequences.

In certain embodiments, the binding molecules or antigen binding domains can comprise portions of “humanized” forms of nonhuman (e.g., camelid, murine, non-human primate) antibodies that include sequences from human immunoglobulins (e.g., recipient antibody) in which the native CDR residues are replaced by residues from the corresponding CDR of a nonhuman species (e.g., donor antibody) such as camelid, mouse, rat, rabbit, or nonhuman primate having the desired specificity, affinity, and capacity. In some instances, one or more FR region residues of the human immunoglobulin sequences are replaced by corresponding nonhuman residues. Furthermore, humanized antibodies can comprise residues that are not found in the recipient antibody or in the donor antibody. These modifications are made to further refine antibody performance. A humanized antibody heavy or light chain can comprise substantially all of at least one or more variable regions, in which all or substantially all of the CDRs correspond to those of a nonhuman immunoglobulin and all or substantially all of the FRs are those of a human immunoglobulin sequence. In certain embodiments, the humanized antibody will comprise at least a portion of an immunoglobulin constant region (Fc), typically that of a human immunoglobulin. For further details, see, Jones et al., Nature 321:522-25 (1986); Riechmann et al., Nature 332:323-29 (1988); Presta, Curr. Op. Struct. Biol. 2:593-96 (1992); Carter et al., Proc. Natl. Acad. Sci. USA 89:4285-89 (1992); U.S. Pat. Nos. 6,800,738; 6,719,971; 6,639,055; 6,407,213; and 6,054,297.

In certain embodiments, the binding molecules or antigen binding domains can comprise portions of a “fully human antibody” or “human antibody,” wherein the terms are used interchangeably herein and refer to an antibody that comprises a human variable region and, for example, a human constant region. The binding molecules may comprise an antibody sequence. In specific embodiments, the terms refer to an antibody that comprises a variable region and constant region of human origin. “Fully human” antibodies, in certain embodiments, can also encompass antibodies which bind polypeptides and are encoded by nucleic acid sequences which are naturally occurring somatic variants of human germline immunoglobulin nucleic acid sequence. The term “fully human antibody” includes antibodies having variable and constant regions corresponding to human germline immunoglobulin sequences as described by Kabat et al. (See Kabat et al. (1991) Sequences of Proteins of Immunological Interest, Fifth Edition, U.S. Department of Health and Human Services, NIH Publication No. 91-3242). A “human antibody” is one that possesses an amino acid sequence which corresponds to that of an antibody produced by a human and/or has been made using any of the techniques for making human antibodies. This definition of a human antibody specifically excludes a humanized antibody comprising non-human antigen-binding residues. Human antibodies can be produced using various techniques known in the art, including phage-display libraries (Hoogenboom and Winter, J. Mol. Biol. 227:381 (1991); Marks et al., J. Mol. Biol. 222:581 (1991)) and yeast display libraries (Chao et al., Nature Protocols 1:755-68 (2006)). Also available for the preparation of human monoclonal antibodies are methods described in Cole et al., Monoclonal Antibodies and Cancer Therapy 77 (1985); Boerner et al., J. Immunol. 147(1): 86-95 (1991); and van Dijk and van de Winkel, Curr. Opin. Pharmacol. 5:368-74 (2001). Human antibodies can be prepared by administering the antigen to a transgenic animal that has been modified to produce such antibodies in response to antigenic challenge, but whose endogenous loci have been disabled, e.g., mice (see, e.g., Jakobovits, Curr. Opin. Biotechnol. 6(5):561-66 (1995); Brüggemann and Taussing, Curr. Opin. Biotechnol. 8(4):455-58 (1997); and U.S. Pat. Nos. 6,075,181 and 6,150,584 regarding XENOMOUSE™ technology). See also, for example, Li et al., Proc. Natl. Acad. Sci. USA 103:3557-62 (2006) regarding human antibodies generated via a human B-cell hybridoma technology.

In certain embodiments, the binding molecules or antigen binding domains can comprise portions of a “recombinant human antibody,” wherein the phrase includes human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse or cow) that is transgenic and/or transchromosomal for human immunoglobulin genes (see, e.g., Taylor, L. D. et al., Nucl. Acids Res. 20:6287-6295 (1992)) or antibodies prepared, expressed, created or isolated by any other means that involves splicing of human immunoglobulin gene sequences to other DNA sequences. Such recombinant human antibodies can have variable and constant regions derived from human germline immunoglobulin sequences (See Kabat, E. A. et al. (1991) Sequences of Proteins of Immunological Interest, Fifth Edition, U.S. Department of Health and Human Services, NIH Publication No. 91-3242). In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

In certain embodiments, the binding molecules or antigen binding domains can comprise a portion of a “monoclonal antibody,” wherein the term as used herein refers to an antibody obtained from a population of substantially homogeneous antibodies, e.g., the individual antibodies comprising the population are identical except for possible naturally occurring mutations that may be present in minor amounts or well-known post-translational modifications such as amino acid isomerization or deamidation, methionine oxidation or asparagine or glutamine deamidation, each monoclonal antibody will typically recognize a single epitope on the antigen. In specific embodiments, a “monoclonal antibody,” as used herein, is an antibody produced by a single hybridoma or other cell. The term “monoclonal” is not limited to any particular method for making the antibody. For example, the monoclonal antibodies useful in the present disclosure may be prepared by the hybridoma methodology first described by Kohler et al., Nature 256:495 (1975), or may be made using recombinant DNA methods in bacterial or eukaryotic animal or plant cells (see, e.g., U.S. Pat. No. 4,816,567). The “monoclonal antibodies” may also be isolated from phage antibody libraries using the techniques described in Clackson et al., Nature 352:624-28 (1991) and Marks et al., J. Mol. Biol. 222:581-97 (1991), for example. Other methods for the preparation of clonal cell lines and of monoclonal antibodies expressed thereby are well known in the art. See, e.g., Short Protocols in Molecular Biology (Ausubel et al. eds., 5th ed. 2002).

A typical 4-chain antibody unit is a heterotetrameric glycoprotein composed of two identical light (L) chains and two identical heavy (H) chains. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (VH) followed by three constant domains (CH) for each of the α and γ chains and four CH domains for μ and ε isotypes. Each L chain has at the N-terminus, a variable domain (VL) followed by a constant domain (CL) at its other end. The VL is aligned with the VH, and the CL is aligned with the first constant domain of the heavy chain (CH1). Particular amino acid residues are believed to form an interface between the light chain and heavy chain variable domains. The pairing of a VH and VL together forms a single antigen-binding site. For the structure and properties of the different classes of antibodies, see, for example, Basic and Clinical Immunology 71 (Stites et al. eds., 8th ed. 1994); and Immunobiology (Janeway et al. eds., 5th ed. 2001).

The term “Fab” or “Fab region” refers to an antibody region that binds to antigens. A conventional IgG usually comprises two Fab regions, each residing on one of the two arms of the Y-shaped IgG structure. Each Fab region is typically composed of one variable region and one constant region of each of the heavy and the light chain. More specifically, the variable region and the constant region of the heavy chain in a Fab region are VH and CH1 regions, and the variable region and the constant region of the light chain in a Fab region are VL and CL regions. The VH, CH1, VL, and CL in a Fab region can be arranged in various ways to confer an antigen binding capability according to the present disclosure. For example, VH and CH1 regions can be on one polypeptide, and VL and CL regions can be on a separate polypeptide, similarly to a Fab region of a conventional IgG. Alternatively, VH, CH1, VL and CL regions can all be on the same polypeptide and oriented in different orders as described in more detail below.

The term “variable region,” “variable domain,” “V region,” or “V domain” refers to a portion of the light or heavy chains of an antibody that is generally located at the amino-terminal of the light or heavy chain and has a length of about 120 to 130 amino acids in the heavy chain and about 100 to 110 amino acids in the light chain, and are used in the binding and specificity of each particular antibody for its particular antigen. The variable region of the heavy chain may be referred to as “VH.” The variable region of the light chain may be referred to as “VL.” The term “variable” refers to the fact that certain segments of the variable regions differ extensively in sequence among antibodies. The V region mediates antigen binding and defines specificity of a particular antibody for its particular antigen. However, the variability is not evenly distributed across the 110-amino acid span of the variable regions. Instead, the V regions consist of less variable (e.g., relatively invariant) stretches called framework regions (FRs) of about 15-30 amino acids separated by shorter regions of greater variability (e.g., extreme variability) called “hypervariable regions” that are each about 9-12 amino acids long. The variable regions of heavy and light chains each comprise four FRs, largely adopting a β sheet configuration, connected by three hypervariable regions, which form loops connecting, and in some cases form part of, the β sheet structure. The hypervariable regions in each chain are held together in close proximity by the FRs and, with the hypervariable regions from the other chain, contribute to the formation of the antigen-binding site of antibodies (see, e.g., Kabat et al., Sequences of Proteins of Immunological Interest (5th ed. 1991)). The constant regions are not involved directly in binding an antibody to an antigen, but exhibit various effector functions, such as participation of the antibody in antibody dependent cellular cytotoxicity (ADCC) and complement dependent cytotoxicity (CDC). The variable regions differ extensively in sequence between different antibodies. In specific embodiments, the variable region is a human variable region.

The term “variable region residue numbering according to Kabat” or “amino acid position numbering as in Kabat”, and variations thereof, refer to the numbering system used for heavy chain variable regions or light chain variable regions of the compilation of antibodies in Kabat et al., supra. Using this numbering system, the actual linear amino acid sequence may contain fewer or additional amino acids corresponding to a shortening of, or insertion into, an FR or CDR of the variable domain. For example, a heavy chain variable domain may include a single amino acid insert (residue 52a according to Kabat) after residue 52 and three inserted residues (e.g., residues 82a, 82b, and 82c, etc. according to Kabat) after residue 82. The Kabat numbering of residues may be determined for a given antibody by alignment at regions of homology of the sequence of the antibody with a “standard” Kabat numbered sequence. The Kabat numbering system is generally used when referring to a residue in the variable domain (approximately residues 1-107 of the light chain and residues 1-113 of the heavy chain) (e.g., Kabat et al., supra). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG 1 EU antibody. Other numbering systems have been described, for example, by AbM, Chothia, Contact, IMGT, and AHon.

The term “heavy chain” when used in reference to an antibody refers to a polypeptide chain of about 50-70 kDa, wherein the amino-terminal portion includes a variable region of about 120 to 130 or more amino acids, and a carboxy-terminal portion includes a constant region. The constant region can be one of five distinct types, (e.g., isotypes) referred to as alpha (α), delta (δ), epsilon (ε), gamma (γ), and mu (μ), based on the amino acid sequence of the heavy chain constant region. The distinct heavy chains differ in size: α, δ, and γ contain approximately 450 amino acids, while μ and ε contain approximately 550 amino acids. When combined with a light chain, these distinct types of heavy chains give rise to five well known classes (e.g., isotypes) of antibodies, IgA, IgD, IgE, IgG, and IgM, respectively, including four subclasses of IgG, namely IgG1, IgG2, IgG3, and IgG4.

The term “light chain” when used in reference to an antibody refers to a polypeptide chain of about 25 kDa, wherein the amino-terminal portion includes a variable region of about 100 to about 110 or more amino acids, and a carboxy-terminal portion includes a constant region. The approximate length of a light chain is 211 to 217 amino acids. There are two distinct types, referred to as kappa (κ) or lambda (λ) based on the amino acid sequence of the constant domains.

As used herein, the terms “hypervariable region,” “HVR,” “Complementarity Determining Region,” and “CDR” are used interchangeably. A “CDR” refers to one of three hypervariable regions (H1, H2 or H3) within the non-framework region of the immunoglobulin (Ig or antibody) VH β-sheet framework, or one of three hypervariable regions (L1, L2 or L3) within the non-framework region of the antibody VL β-sheet framework. CDR1, CDR2 and CDR3 in VH domain are also referred to as HCDR1, HCDR2 and HCDR3, respectively. CDR1, CDR2 and CDR3 in VL domain are also referred to as LCDR1, LCDR2 and LCDR3, respectively. Accordingly, CDRs are variable region sequences interspersed within the framework region sequences.

CDR regions are well known to those skilled in the art and have been defined by well-known numbering systems. For example, the Kabat Complementarity Determining Regions (CDRs) are based on sequence variability and are the most commonly used (see, e.g., Kabat et al., supra; Nick Deschacht et al., J Immunol 2010; 184:5696-5704). Chothia refers instead to the location of the structural loops (see, e.g., Chothia and Lesk, J. Mol. Biol. 196:901-17 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software (see, e.g., Antibody Engineering Vol. 2 (Kontermann and Dübel eds., 2d ed. 2010)). The “contact” hypervariable regions are based on an analysis of the available complex crystal structures. Another universal numbering system that has been developed and widely adopted is ImMunoGeneTics (IMGT) Information System® (Lafranc et al., Dev. Comp. Immunol. 27(1):55-77 (2003)). IMGT is an integrated information system specializing in immunoglobulins (IG), T-cell receptors (TCR), and major histocompatibility complex (MHC) of human and other vertebrates. Herein, the CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues are readily identified. This information can be used in grafting and replacement of CDR residues from immunoglobulins of one species into an acceptor framework from, typically, a human antibody. An additional numbering system (AHon) has been developed by Honegger and Plückthun, J. Mol. Biol. 309:657-70 (2001). Correspondence between the numbering system, including, for example, the Kabat numbering and the IMGT unique numbering system, is well known to one skilled in the art (see, e.g., Kabat, supra; Chothia and Lesk, supra; Martin, supra; Lefranc et al., supra). The residues from each of these hypervariable regions or CDRs are exemplified in Table 1 below.

TABLE 1
Exemplary CDRs According to Various Numbering Systems

| Loop | Kabat | AbM | Chothia | Contact | IMGT |
|---|---|---|---|---|---|
| CDR L1 | L24--L34 | L24--L34 | L26--L32 | L30--L36 | L27--L38 |
| | | | or | | |
| | | | L24--L34 | | |
| CDR L2 | L50--L56 | L50--L56 | L50--L52 | L46--L55 | L56--L65 |
| | | | or | | |
| | | | L50--L56 | | |
| CDR L3 | L89--L97 | L89--L97 | L91--L96 | L89--L96 | L105-L117 |
| | | | or | | |
| | | | L89--L97 | | |
| CDR H1 | H31--H35B | H26--H35B | H26-- | H30--H35B | H27--H38 |
| | (Kabat | | H32..34 | | |
| | Numbering) | | | | |
| CDR H1 | H31--H35 | H26--H35 | H26--H32 | H30--H35 | |
| | (Chothia | | | | |
| | Numbering) | | | | |
| CDR H2 | H50--H65 | H50--H58 | H53--H55 | H47--H58 | H56--H65 |
| | | | or | | |
| | | | H52--H56 | | |
| CDR H3 | H95--H102 | H95--H102 | H96--H101 | H93--H101 | H105-H117 |
| | | | or | | |
| | | | H95--H102 | | |

The boundaries of a given CDR may vary depending on the scheme used for identification. Thus, unless otherwise specified, the terms “CDR” and “complementary determining region” of a given antibody or region thereof, such as a variable region, as well as individual CDRs (e.g., CDR-H1, CDR-H2) of the antibody or region thereof, should be understood to encompass the complementary determining region as defined by any of the known schemes described herein above. In some instances, the scheme for identification of a particular CDR or CDRs is specified, such as the CDR as defined by the IMGT, Kabat, Chothia, or Contact method. In other cases, the particular amino acid sequence of a CDR is given. It should be noted CDR regions may also be defined by a combination of various numbering systems, e.g., a combination of Kabat and Chothia numbering systems, or a combination of Kabat and IMGT numbering systems. Therefore, the term such as “a CDR1 as set forth in a specific VH” includes any CDR1 as defined by the exemplary CDR numbering systems described above, but is not limited thereby. Once a variable region (e.g., a VH or VL) is given, those skilled in the art would understand that CDRs within the region can be defined by different numbering systems or combinations thereof.

Hypervariable regions may comprise “extended hypervariable regions” as follows: 24-36 or 24-34 (L1), 46-56 or 50-56 (L2), and 89-97 or 89-96 (L3) in the VL, and 26-35 or 26-35A (H1), 50-65 or 49-65 (H2), and 93-102, 94-102, or 95-102 (H3) in the VH.

The term “constant region” or “constant domain” refers to a carboxy terminal portion of the light and heavy chain which is not directly involved in binding of the antibody to antigen but exhibits various effector function, such as interaction with the Fc receptor. The term refers to the portion of an immunoglobulin molecule having a more conserved amino acid sequence relative to the other portion of the immunoglobulin, the variable region, which contains the antigen binding site. The constant region may contain the CH1, CH2, and CH3 regions of the heavy chain and the CL region of the light chain.

The term “framework” or “FR” refers to those variable region residues flanking the CDRs. FR residues are present, for example, in chimeric, humanized, human, domain antibodies, diabodies, linear antibodies, and bispecific antibodies. FR residues are those variable domain residues other than the hypervariable region residues or CDR residues.

The term “Fc region” herein is used to define a C-terminal region of an immunoglobulin heavy chain, including, for example, native sequence Fc regions, recombinant Fc regions, and variant Fc regions. Although the boundaries of the Fc region of an immunoglobulin heavy chain might vary, the human IgG heavy chain Fc region is often defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. A “functional Fc region” possesses an “effector function” of a native sequence Fc region. Exemplary “effector functions” include C1q binding; CDC; Fc receptor binding; ADCC; phagocytosis; downregulation of cell surface receptors (e.g., B cell receptor), etc. Such effector functions generally require the Fc region to be combined with a binding region or binding domain (e.g., an antibody variable region or domain) and can be assessed using various assays known to those skilled in the art. A “variant Fc region” comprises an amino acid sequence which differs from that of a native sequence Fc region by virtue of at least one amino acid modification (e.g., substituting, addition, or deletion). In certain embodiments, the variant Fc region has at least one amino acid substitution compared to a native sequence Fc region or to the Fc region of a parent polypeptide, for example, from about one to about ten amino acid substitutions, or from about one to about five amino acid substitutions in a native sequence Fc region or in the Fc region of a parent polypeptide. The variant Fc region herein can possess at least about 80% homology with a native sequence Fc region and/or with an Fc region of a parent polypeptide, or at least about 90% homology therewith, for example, at least about 95% homology therewith.

As used herein, an “epitope” is a term in the art and refers to a localized region of an antigen to which a binding molecule (e.g., an antibody) can specifically bind. An epitope can be a linear epitope or a conformational, non-linear, or discontinuous epitope. In the case of a polypeptide antigen, for example, an epitope can be contiguous amino acids of the polypeptide (a “linear” epitope) or an epitope can comprise amino acids from two or more non-contiguous regions of the polypeptide (a “conformational,” “non-linear” or “discontinuous” epitope). It will be appreciated by one of skill in the art that, in general, a linear epitope may or may not be dependent on secondary, tertiary, or quaternary structure. For example, in some embodiments, a binding molecule binds to a group of amino acids regardless of whether they are folded in a natural three dimensional protein structure. In other embodiments, a binding molecule requires amino acid residues making up the epitope to exhibit a particular conformation (e.g., bend, twist, turn or fold) in order to recognize and bind the epitope.

The term “biparatopic” antigen binding molecule or “biparatopic” polypeptide as used herein shall mean a polypeptide comprising a first immunoglobulin single variable domain and a second immunoglobulin single variable domain as herein defined, wherein these two variable domains are capable of binding to two different epitopes of one antigen.

“Percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

The term “specificity” refers to selective recognition of an antigen binding protein for a particular epitope of an antigen. Natural antibodies, for example, are monospecific. The term “multispecific” as used herein denotes that an antigen binding protein has two or more antigen-binding sites of which at least two bind different antigens. “Bispecific” as used herein denotes that an antigen binding protein has two different antigen-binding specificities. The term “monospecific” antibody as used herein denotes an antigen binding protein that has one or more binding sites each of which bind the same antigen.

The term “valent” as used herein denotes the presence of a specified number of binding sites in an antigen binding protein. A natural antibody for example or a full length antibody has two binding sites and is bivalent. As such, the terms “trivalent”, “tetravalent”, “pentavalent” and “hexavalent” denote the presence of two binding site, three binding sites, four binding sites, five binding sites, and six binding sites, respectively, in an antigen binding protein.

The terms “polypeptide” and “peptide” and “protein” are used interchangeably herein and refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification. Also included within the definition are, for example, polypeptides containing one or more analogs of an amino acid, including but not limited to, unnatural amino acids, as well as other modifications known in the art. It is understood that, because the polypeptides of this disclosure may be based upon antibodies or other members of the immunoglobulin superfamily, in certain embodiments, a “polypeptide” can occur as a single chain or as two or more associated chains.

“Polynucleotide” or “nucleic acid,” as used interchangeably herein, refers to polymers of nucleotides of any length and includes DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. “Oligonucleotide,” as used herein, refers to short, generally single-stranded, synthetic polynucleotides that are generally, but not necessarily, fewer than about 200 nucleotides in length. The terms “oligonucleotide” and “polynucleotide” are not mutually exclusive. The description above for polynucleotides is equally and fully applicable to oligonucleotides. A cell that produces a binding molecule of the present disclosure may include a parent hybridoma cell, as well as bacterial and eukaryotic host cells into which nucleic acids encoding the antibodies have been introduced. Unless specified otherwise, the left-hand end of any single-stranded polynucleotide sequence disclosed herein is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA transcript that are 5′ to the 5′ end of the RNA transcript are referred to as “upstream sequences”; sequence regions on the DNA strand having the same sequence as the RNA transcript that are 3′ to the 3′ end of the RNA transcript are referred to as “downstream sequences.”

An “isolated nucleic acid” is a nucleic acid, for example, an RNA, DNA, or a mixed nucleic acids, which is substantially separated from other genome DNA sequences as well as proteins or complexes such as ribosomes and polymerases, which naturally accompany a native sequence. An “isolated” nucleic acid molecule is one which is separated from other nucleic acid molecules which are present in the natural source of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. In a specific embodiment, one or more nucleic acid molecules encoding an antibody as described herein are isolated or purified. The term embraces nucleic acid sequences that have been removed from their naturally occurring environment, and includes recombinant or cloned DNA isolates and chemically synthesized analogues or analogues biologically synthesized by heterologous systems. A substantially pure molecule may include isolated forms of the molecule. Specifically, an “isolated” nucleic acid molecule encoding an antibody described herein is a nucleic acid molecule that is identified and separated from at least one contaminant nucleic acid molecule with which it is ordinarily associated in the environment in which it was produced.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

As used herein, the term “operatively linked,” and similar phrases (e.g., genetically fused), when used in reference to nucleic acids or amino acids, refer to the operational linkage of nucleic acid sequences or amino acid sequence, respectively, placed in functional relationships with each other. For example, an operatively linked promoter, enhancer elements, open reading frame, 5′ and 3′ UTR, and terminator sequences result in the accurate production of a nucleic acid molecule (e.g., RNA). In some embodiments, operatively linked nucleic acid elements result in the transcription of an open reading frame and ultimately the production of a polypeptide (i.e., expression of the open reading frame). As another example, an operatively linked peptide is one in which the functional domains are placed with appropriate distance from each other to impart the intended function of each domain.

The term “vector” refers to a substance that is used to carry or include a nucleic acid sequence, including for example, a nucleic acid sequence encoding a binding molecule (e.g., an antibody) as described herein, in order to introduce a nucleic acid sequence into a host cell. Vectors applicable for use include, for example, expression vectors, plasmids, phage vectors, viral vectors, episomes, and artificial chromosomes, which can include selection sequences or markers operable for stable integration into a host cell's chromosome. Additionally, the vectors can include one or more selectable marker genes and appropriate expression control sequences. Selectable marker genes that can be included, for example, provide resistance to antibiotics or toxins, complement auxotrophic deficiencies, or supply critical nutrients not in the culture media. Expression control sequences can include constitutive and inducible promoters, transcription enhancers, transcription terminators, and the like, which are well known in the art. When two or more nucleic acid molecules are to be co-expressed (e.g., both an antibody heavy and light chain or an antibody VH and VL), both nucleic acid molecules can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The introduction of nucleic acid molecules into a host cell can be confirmed using methods well known in the art. Such methods include, for example, nucleic acid analysis such as Northern blots or polymerase chain reaction (PCR) amplification of mRNA, immunoblotting for expression of gene products, or other suitable analytical methods to test the expression of an introduced nucleic acid sequence or its corresponding gene product. It is understood by those skilled in the art that the nucleic acid molecules are expressed in a sufficient amount to produce a desired product and it is further understood that expression levels can be optimized to obtain sufficient expression using methods well known in the art.

The term “host” as used herein refers to an animal, such as a mammal (e.g., a human).

The term “host cell” as used herein refers to a particular subject cell that may be transfected with a nucleic acid molecule and the progeny or potential progeny of such a cell. Progeny of such a cell may not be identical to the parent cell transfected with the nucleic acid molecule due to mutations or environmental influences that may occur in succeeding generations or integration of the nucleic acid molecule into the host cell genome.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The term “pharmaceutically acceptable” as used herein means being approved by a regulatory agency of the Federal or a state government, or listed in United States Pharmacopeia, European Pharmacopeia, or other generally recognized Pharmacopeia for use in animals, and more particularly in humans.

“Excipient” means a pharmaceutically-acceptable material, composition, or vehicle, such as a liquid or solid filler, diluent, solvent, or encapsulating material. Excipients include, for example, encapsulating materials or additives such as absorption accelerators, antioxidants, binders, buffers, carriers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents and mixtures thereof. The term “excipient” can also refer to a diluent, adjuvant (e.g., Freunds' adjuvant (complete or incomplete) or vehicle.

In some embodiments, excipients are pharmaceutically acceptable excipients. Examples of pharmaceutically acceptable excipients include buffers, such as phosphate, citrate, and other organic acids; antioxidants, including ascorbic acid; low molecular weight (e.g., fewer than about 10 amino acid residues) polypeptide; proteins, such as serum albumin, gelatin, or immunoglobulins; hydrophilic polymers, such as polyvinylpyrrolidone; amino acids, such as glycine, glutamine, asparagine, arginine, or lysine; monosaccharides, disaccharides, and other carbohydrates, including glucose, mannose, or dextrins; chelating agents, such as EDTA; sugar alcohols, such as mannitol or sorbitol; salt-forming counterions, such as sodium; and/or nonionic surfactants, such as TWEEN™, polyethylene glycol (PEG), and PLURONICS™. Other examples of pharmaceutically acceptable excipients are described in Remington and Gennaro, Remington's Pharmaceutical Sciences (18th ed. 1990).

In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Lippincott Williams & Wilkins: Philadelphia, PA, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, FL, 2009. In some embodiments, pharmaceutically acceptable excipients are nontoxic to the cell or mammal being exposed thereto at the dosages and concentrations employed. In some embodiments, a pharmaceutically acceptable excipient is an aqueous pH buffered solution.

In some embodiments, excipients are sterile liquids, such as water and oils, including those of petroleum, animal, vegetable, or synthetic origin, such as peanut oil, soybean oil, mineral oil, sesame oil, and the like. Water is an exemplary excipient when a composition (e.g., a pharmaceutical composition) is administered intravenously. Saline solutions and aqueous dextrose and glycerol solutions can also be employed as liquid excipients, particularly for injectable solutions. An excipient can also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition, if desired, can also contain minor amounts of wetting or emulsifying agents, or pH buffering agents. Compositions can take the form of solutions, suspensions, emulsion, tablets, pills, capsules, powders, sustained-release formulations, and the like. Oral compositions, including formulations, can include standard excipients such as pharmaceutical grades of mannitol, lactose, starch, magnesium stearate, sodium saccharine, cellulose, magnesium carbonate, etc.

Compositions, including pharmaceutical compounds, may contain a binding molecule (e.g., an antibody), for example, in isolated or purified form, together with a suitable amount of excipients.

The term “effective amount” or “therapeutically effective amount” as used herein refers to the amount of an antibody or a therapeutic molecule comprising an agent and the antibody or pharmaceutical composition provided herein which is sufficient to result in the desired outcome.

The terms “subject” and “patient” may be used interchangeably. As used herein, in certain embodiments, a subject is a mammal, such as a non-primate or a primate (e.g., human). In specific embodiments, the subject is a human. In one embodiment, the subject is a mammal, e.g., a human, diagnosed with a disease or disorder. In another embodiment, the subject is a mammal, e.g., a human, at risk of developing a disease or disorder.

“Administer” or “administration” refers to the act of injecting or otherwise physically delivering a substance as it exists outside the body into a patient, such as by mucosal, intradermal, intravenous, intramuscular delivery, and/or any other method of physical delivery described herein or known in the art.

As used herein, the terms “treat,” “treatment” and “treating” refer to the reduction or amelioration of the progression, severity, and/or duration of a disease or condition resulting from the administration of one or more therapies. Treating may be determined by assessing whether there has been a decrease, alleviation and/or mitigation of one or more symptoms associated with the underlying disorder such that an improvement is observed with the patient, despite that the patient may still be afflicted with the underlying disorder. The term “treating” includes both managing and ameliorating the disease. The terms “manage,” “managing,” and “management” refer to the beneficial effects that a subject derives from a therapy which does not necessarily result in a cure of the disease.

The terms “prevent,” “preventing,” and “prevention” refer to reducing the likelihood of the onset (or recurrence) of a disease, disorder, condition, or associated symptom(s) (e.g., diabetes or a cancer).

As used herein, “delaying” the development of cancer means to defer, hinder, slow, retard, stabilize, and/or postpone development of the disease. This delay can be of varying lengths of time, depending on the history of the disease and/or individual being treated. As is evident to one skilled in the art, a sufficient or significant delay can, in effect, encompass prevention, in that the individual does not develop the disease. A method that “delays” development of cancer is a method that reduces probability of disease development in a given time frame and/or reduces the extent of the disease in a given time frame, when compared to not using the method. Such comparisons are typically based on clinical studies, using a statistically significant number of individuals. Cancer development can be detectable using standard methods, including, but not limited to, computerized axial tomography (CAT Scan), Magnetic Resonance Imaging (MRI), abdominal ultrasound, clotting tests, arteriography, or biopsy. Development may also refer to cancer progression that may be initially undetectable and includes occurrence, recurrence, and onset.

The term “HER2” (also known as HER2/neu and ErbB-2) stands for “Human Epidermal growth factor Receptor 2”. As used herein, it is intended to include variants, isoforms and species homologs of HER2.

The term “HER2 associated disease or disorder” as used herein refers to a disease or disorder that comprises a cell or tissue in which HER2 is expressed or overexpressed. In some embodiments, HER2 associated disease or disorder comprises a cell on which HER2 is abnormally expressed. In other embodiments, HER2 associated disease or disorder comprises a cell in or on which HER2 is deficient in at least one of its activities.

The “blood-brain barrier” or “BBB” refers a physiological barrier between the peripheral circulation and the brain and spinal cord which is formed by tight junctions within the brain capillary endothelial plasma membranes, creating a tight barrier that restricts the transport of molecules into the brain. The BBB can restrict the transport of even very small molecules such as urea (60 Daltons) into the brain. Examples of the BBB include the BBB within the brain, the blood-spinal cord barrier within the spinal cord, and the blood-retinal barrier within the retina, all of which are contiguous capillary barriers within the CNS. The BBB also encompasses the blood-CSF barrier (choroid plexus) where the barrier is comprised of ependymal cells rather than capillary endothelial cells.

A “blood-brain barrier receptor” (abbreviated “R/BBB” herein) is an extracellular membrane-linked receptor protein expressed on brain endothelial cells which is capable of transporting molecules across the BBB or be used to transport exogenous administrated molecules. Examples of R/BBB include, but are not limited to, Large neutral Amino acid Transporter (LAT) complex, including CD98 component, transferrin receptor (TfR), insulin receptor, insulin-like growth factor receptor (IGF-R), low density lipoprotein receptors including without limitation low density lipoprotein receptor-related protein 1 (LRP1) and low density lipoprotein receptor-related protein 8 (LRP8), and heparin-binding epidermal growth factor-like growth factor (HB-EGF). An exemplary R/BBB herein is CD98.

The term “CD98” or “CD98 hc” as used herein, refers to an integral membrane protein consisting of a cluster of differentiation 98 heavy chain (CD98 hc) that links to any of multiple light chain by a disulfide bond. When associated with LAT1 or LAT2, the heterodimer transporter complexes behave as obligatory amino acid exchangers. CD98 hc has a molecular weight of about 80 kDa. Preferably, the CD98 hc is a human CD98 hc (huCD98 hc). hcCD98 hc is encoded by the SLC3A2 gene.

The terms “about” and “approximately” mean within 20%, within 15%, within 10%, within 9%, within 8%, within 7%, within 6%, within 5%, within 4%, within 3%, within 2%, within 1%, or less of a given value or range.

As used in the present disclosure and claims, the singular forms “a”, “an” and “the” include plural forms unless the context clearly dictates otherwise.

It is understood that wherever embodiments are described herein with the term “comprising” otherwise analogous embodiments described in terms of “consisting of” and/or “consisting essentially of” are also provided. It is also understood that wherever embodiments are described herein with the phrase “consisting essentially of” otherwise analogous embodiments described in terms of “consisting of” are also provided.

The term “between” as used in a phrase as such “between A and B” or “between A-B” refers to a range including both A and B.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Multispecific Antibodies with Tissue Targeting Moiety

In one general aspect, the application relates to an optimized platform for a particular tissue delivery. In one aspect, the platform utilizes a binding molecule, in particular, an antibody or antigen-binding fragment thereof that binds to a target that expresses on a particular tissue.

In one aspect, provided herein is a multispecific antibody comprising at least one of a first antigen-binding region and a second antigen-binding region each capable of binding specifically to human epidermal growth factor receptor 2 (HER2), and a third antigen-binding region capable of binding specifically to CD98.

HER2 (also known as ErbB2 or Neu; UniProtKB/Swiss-Prot No. P04626) consists of 1233 amino acids and is structurally similar to EGFR with an extracellular domain consisting of four subdomains I-IV, a transmembrane domain, a juxtamembrane domain, an intracellular cytoplasmic tyrosine kinase and a regulatory C-terminal domain (Yamamoto et al. (1986) Nature 319:230-234). HER2 is activated via formation of heteromeric complexes with other ErbB family members and thereby indirectly regulated by EGFR and HER3 ligands (reviewed in Yarden et al. (2001) Nat Rev Mol Cell Biol. 2:127-137). HER2 is the preferred heterodimerization partner of the three other ErbB receptors (Graus-Porta et al. (1997) EMBO J 16:1647-1655; Tzahar et al. (1996) Mol Cell Biol. 16:5276-5287), enhancing the affinity of the other ErbB receptors for their ligands by slowing down the rate of ligand-receptor complex dissociation, whereby HER2 enhances and prolongs signaling (Pedersen et al. (2009) Mol Cancer Res. 7:275-284). Heterodimerization of HER2 and another ligand-bound receptor of the ErbB family induces cross-phosphorylation, leading to phosphorylation of the C-terminal amino acids. These in turns serve as scaffolds for signaling molecules (King et al. (1988) EMBO J 7:1647-1651). The most active HER2 heterodimer is the HER2-HER3 complex (Pinkas-Kramarski et al. (1996) EMBO J 15:2452-2467), where HER2 complements the kinase-deficient HER3 by providing an active kinase (Guy et al. (1994) Proc Natl Acad Sci USA 91:8132-8136.). In contrast to EGFR, HER2 is internalization resistant (Hommelgaard et al. (2004) Mol Biol Cell 15:1557-1567), escaping lysosomal degradation and thereby remaining at the plasma membrane.

In certain embodiments, an antigen-binding region provided herein comprises one or more CDR sequences. CDR sequences can be determined according to well-known numbering systems. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering.

In some embodiments, the first antigen-binding region provided herein comprises a first heavy chain variable region (VH1) comprising a heavy chain complementarity determining region 1 (HCDR1), HCDR2, and HCDR3 as set forth in the HC comprising the amino acid sequence of SEQ ID NO:1, and a first light chain variable region (VL1) comprising the LCDR1, LCDR2, and LCDR3 as set forth in the LC comprising the amino acid sequence of SEQ ID NO:2. In certain embodiments, the first heavy chain variable region (VH1) comprises a heavy chain complementarity determining region 1 (HCDR1), a HCDR2, and a HCDR3 comprising the amino acid sequences of SEQ ID NOs: 5, 6 and 7, respectively; and the first light chain variable region (VL1) comprises a light chain complementarity determining region 1 (LCDR1), a LCDR2, and a LCDR3 comprising the amino acid sequences of SEQ ID NOs: 8, 9 and 10, respectively.

In certain embodiments, the VH1 comprises an amino acid sequence at least 90%, at least 91%, at least 92%, at least 93%, at least 94%, at least 95%, at least 96%, at least 97%, at least 98%, or at least 99% identical to the VH of the HC sequence identified as SEQ ID NO: 1; and the VL1 comprises an amino acid sequence at least 90%, at least 91%, at least 92%, at least 93%, at least 94%, at least 95%, at least 96%, at least 97%, at least 98%, or at least 99% identical to the VL of the LC sequence identified as SEQ ID NO: 2. In certain embodiments, the VH1 an amino acid sequence identical to the VH of the HC sequence identified as SEQ ID NO: 1; and the VL1 comprises an amino acid sequence identical to the VL of the LC sequence identified as SEQ ID NO: 2.

In some embodiments, the second antigen-binding region provided herein comprises a first heavy chain variable region (VH2) comprising a heavy chain complementarity determining region 1 (HCDR1), HCDR2, and HCDR3 as set forth in the HC comprising the amino acid sequence of SEQ ID NO:11, and a second light chain variable region (VL2) comprising the LCDR1, LCDR2, and LCDR3 as set forth in the LC comprising the amino acid sequence of SEQ ID NO: 12. In certain embodiments, the second heavy chain variable region (VH1) comprises a heavy chain complementarity determining region 1 (HCDR1), a HCDR2, and a HCDR3 comprising the amino acid sequences of SEQ ID NOs: 13, 14 and 15, respectively; and the first light chain variable region (VL1) comprises a light chain complementarity determining region 1 (LCDR1), a LCDR2, and a LCDR3 comprising the amino acid sequences of SEQ ID NOs: 16, 17 and 18, respectively.

In certain embodiments, the VH1 comprises an amino acid sequence at least 90%, at least 91%, at least 92%, at least 93%, at least 94%, at least 95%, at least 96%, at least 97%, at least 98%, or at least 99% identical to the VH of the HC sequence identified as SEQ ID NO: 1; and the VL1 comprises an amino acid sequence at least 90%, at least 91%, at least 92%, at least 93%, at least 94%, at least 95%, at least 96%, at least 97%, at least 98%, or at least 99% identical to the VL of the LC sequence identified as SEQ ID NO: 2. In certain embodiments, the VH1 an amino acid sequence identical to the VH of the HC sequence identified as SEQ ID NO: 1; and the VL1 comprises an amino acid sequence identical to the VL of the LC sequence identified as SEQ ID NO: 2.

The determination of percent identity between two sequences (e.g., amino acid sequences or nucleic acid sequences) can be accomplished using a mathematical algorithm. A non-limiting example of a mathematical algorithm utilized for the comparison of two sequences is the algorithm of Karlin and Altschul, Proc. Natl. Acad. Sci. U.S.A. 87:2264 2268 (1990), modified as in Karlin and Altschul, Proc. Natl. Acad. Sci. U.S.A. 90:5873 5877 (1993). Such an algorithm is incorporated into the NBLAST and XBLAST programs of Altschul et al., J. Mol. Biol. 215:403 (1990). BLAST nucleotide searches can be performed with the NBLAST nucleotide program parameters set, e.g., for score=100, word length=12 to obtain nucleotide sequences homologous to a nucleic acid molecules described herein. BLAST protein searches can be performed with the XBLAST program parameters set, e.g., to score 50, word length=3 to obtain amino acid sequences homologous to a protein molecule described herein. To obtain gapped alignments for comparison purposes, Gapped BLAST can be utilized as described in Altschul et al., Nucleic Acids Res. 25:3389 3402 (1997). Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see, e.g., National Center for Biotechnology Information (NCBI) on the worldwide web, ncbi.nlm.nih.gov). Another non-limiting example of a mathematical algorithm utilized for the comparison of sequences is the algorithm of Myers and Miller, CABIOS 4:11-17 (1998). Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

In some embodiments, the antibody provide herein contains substitutions (e.g., conservative substitutions), insertions, or deletions relative to the reference sequence, but the anti-HER2 antibody comprising that sequence retains the ability to bind to HER2. In some embodiments, a total of 1 to 10 amino acids have been substituted, inserted and/or deleted in a reference amino acid sequence. In some embodiments, substitutions, insertions, or deletions occur in regions outside the CDRs (i.e., in the FRs). Optionally, the anti-HER2 antibody or antigen-binding region provided herein includes post-translational modifications of a reference sequence.

In some embodiments, functional epitopes can be mapped, e.g., by combinatorial alanine scanning, to identify amino acids in the HER2 protein that are necessary for interaction with anti-HER2 antibodies provided herein. In some embodiments, conformational and crystal structure of anti-HER2 antibody bound to HER2 may be employed to identify the epitopes. In some embodiments, the present disclosure provides an antibody that specifically binds to the same epitope as any of the anti-HER2 antibodies provided herein.

In certain embodiments, the multispecific antibodies of the invention comprise a third antigen-binding portion thereof that binds to a primate CD98, such as a human CD98 or a monkey CD98, and the antibody or an antigen binding fragment thereof is optimized for delivering an agent to the brain of a subject in need thereof. The inventors of the present invention surprisingly discovered a more nuanced relationship between affinity and transcytosis efficiency than what has been previously described, with influence from both on- and off-rates impacting brain concentration. In particular, a neutral off-rate that is neither too fast nor too slow is required for optimal brain PK and PD of an agent (such as an mAb) to be efficiently delivered by the anti-CD98 antibody or antigen binding fragment thereof. Anti-CD98 antibodies and antigen-binding fragments thereof are described in International Publication No. WO 2021205361, which is incorporated herein by reference in its entirety.

In certain embodiments, the third antigen-binding region comprises a first single chain variable fragment (scFv1) having: a third heavy chain variable region (VH3) comprising a HCDR1, a HCDR2, and a HCDR3, and a third light chain variable region (VL3) comprising a LCDR1, a LCDR2, and a LCDR3, wherein the HCDR1, HCDR2, HCDR3, LCDR1, LCDR2 and LCDR3 are identical to the respective CDRs of a scFv identified in Table 2.

In certain embodiments, the third antigen-binding region comprises a first single chain variable fragment (scFv1) having: a third heavy chain variable region (VH3) comprising a heavy chain complementarity determining region 1 (HCDR1), a HCDR2, and a HCDR3, and a third light chain variable region (VL3) comprising a light chain complementarity determining region 1 (LCDR1), a LCDR2, and a LCDR3, wherein the HCDR1, HCDR2, HCDR3, LCDR1, LCDR2 and LCDR3, respectively, have any of the amino acid sequences identified Table 2.

TABLE 2
Exemplary anti-CD98 antibodies or antigen
binding fragments thereof

| | Se- | | | | | | |
|---|---|---|---|---|---|---|---|
| Name | quence | HCDR1 | HCDR2 | HCDR3 | LCDR1 | LCDR2 | LCDR3 |
| CD98hc1 | SEQ ID | SEQ ID | SEQ ID | SEQ ID | SEQ ID | SEQ ID | SEQ ID |
| | NO: 19 | NO: 20 | NO: 21 | NO: 22 | NO: 23 | NO: 24 | NO: 25 |
| | | GGSIS | IYYSG | ARAGP | SSDVD | DVSNR | SSFTS |
| | | SGAYY | ST | KWELW | NYNY | PS | SSTWV |
| | | W | | FDY | | | |
| CD98hc2 | SEQ ID | SEQ ID | SEQ ID | SEQ ID | SEQ ID | SEQ ID | SEQ ID |
| | NO: 26 | NO: 27 | NO: 28 | NO: 29 | NO: 30 | NO: 31 | NO: 32 |
| | | GFTFS | ISYDG | ARAPS | QSVLF | WAS | QQYYS |
| | | SYG | SNK | SFYFD | SSNNK | | TPPT |
| | | | | Y | NY | | |
| CD98hc2- | SEQ ID | SEQ ID | SEQ ID | SEQ ID | SEQ ID | SEQ ID | SEQ ID |
| H | NO: 33 | NO: 27 | NO: 28 | NO: 29 | NO: 30 | NO: 31 | NO: 32 |
| | | GFTFS | ISYDG | ARAPS | QSVLF | WAS | QQYYS |
| | | SYG | SNK | SFYFD | SSNNK | | TPPT |
| | | | | Y | NY | | |
| CD98hc2 | SEQ ID | SEQ ID | SEQ ID | SEQ ID | SEQ ID | SEQ ID | SEQ ID |
| sp | NO: 34 | NO: 27 | NO: 28 | NO: 29 | NO: 30 | NO: 31 | NO: 32 |
| | | GFTFS | ISYDG | ARAPS | QSVLF | WAS | QQYYS |
| | | SYG | SNK | SFYFD | SSNNK | | TPPT |
| | | | | Y | NY | | |
| CD98hc2 | SEQ ID | SEQ ID | SEQ ID | SEQ ID | SEQ ID | SEQ ID | SEQ ID |
| sp-H | NO: 35 | NO: 27 | NO: 28 | NO: 29 | NO: 30 | NO: 31 | NO: 32 |
| | | GFTFS | ISYDG | ARAPS | QSVLF | WAS | QQYYS |
| | | SYG | SNK | SFYFD | SSNNK | | TPPT |
| | | | | Y | NY | | |
| CD98hc3 | SEQ ID | SEQ ID | SEQ ID | SEQ ID | SEQ ID | SEQ ID | SEQ ID |
| | NO: 36 | NO: 37 | NO: 38 | NO: 39 | NO: 40 | NO: 41 | NO: 42 |
| | | GGSIS | IYNSG | ARETY | QNVGI | SAA | QQYNS |
| | | SSY | ST | YFDY | N | | YPLT |
| CD98hc4 | SEQ ID | SEQ ID | SEQ ID | SEQ ID | SEQ ID | SEQ ID | SEQ ID |
| | NO: 43 | NO: 44 | NO: 45 | NO: 46 | NO: 47 | NO: 31 | NO: 48 |
| | | GYTFT | INPSG | ARDPN | QSVLY | WAS | HQYYS |
| | | SYY | GTT | DYGGP | RSNNK | | TPLT |
| | | | | KYFPH | NY | | |
| CD98hc5 | SEQ ID | SEQ ID | SEQ ID | SEQ ID | SEQ ID | SEQ ID | SEQ ID |
| | NO: 49 | NO: 50 | NO: 51 | NO: 52 | NO: 53 | NO: 31 | NO: 54 |
| | | EYTFT | INPSG | ARAGY | QSVLY | WAS | QYYST |
| | | SYY | GST | CTINS | ASRNK | | PFT |
| | | | | CFNWF | NY | | |
| | | | | DP | | | |
| CD98hc6 | SEQ ID | SEQ ID | SEQ ID | SEQ ID | SEQ ID | SEQ ID | SEQ ID |
| | NO: 55 | NO: 44 | NO: 56 | NO: 57 | NO: 47 | NO: 31 | NO: 58 |
| | | GYTFT | FNPSG | ARGGV | QSVLY | WAS | QQYYG |
| | | SYY | GST | GATGY | RSNNK | | TPFT |
| | | | | FDY | NY | | |
| CD98hc7 | SEQ ID | SEQ ID | SEQ ID | SEQ ID | SEQ ID | SEQ ID | SEQ ID |
| | NO: 59 | NO: 60 | NO: 61 | NO: 62 | NO: 63 | NO: 64 | NO: 65 |
| | | GYTFT | INPNS | LRGKT | QGISS | AAS | QQLNN |
| | | GYY | GGS | FDP | H | | YPLT |
| CD98hc8 | SEQ ID | SEQ ID | SEQ ID | SEQ ID | SEQ ID | SEQ ID | SEQ ID |
| | NO: 66 | NO: 44 | NO: 67 | NO: 68 | NO: 69 | NO: 31 | NO: 70 |
| | | GYTFT | NPSGG | ARAGY | QSVLY | WAS | QQYYS |
| | | SYY | ST | CRSTT | NSNNK | | TPFT |
| | | | | SCFNW | NY | | |
| | | | | FDP | | | |
| CD98hc9 | SEQ ID | SEQ ID | SEQ ID | SEQ ID | SEQ ID | SEQ ID | SEQ ID |
| | NO: 71 | NO: 72 | NO: 73 | NO: 74 | NO: 75 | NO: 76 | NO: 77 |
| | | GFTLS | INSDG | ARWNW | QSLLH | LGS | MQTLE |
| | | SSW | SST | GWYFD | SHGDN | | TPYT |
| | | | | L | Y | | |
| CD98hc10 | SEQ ID | SEQ ID | SEQ ID | SEQ ID | SEQ ID | SEQ ID | SEQ ID |
| | NO: 78 | NO: 79 | NO: 80 | NO: 81 | NO: 82 | NO: 83 | NO: 84 |
| | | GFTFS | INSDG | VREGR | NSNIG | GNS | QSYDS |
| | | SYW | SII | YTGSS | AGYD | | SLTGW |
| | | | | LDY | | | V |
| CD98hc11 | SEQ ID | SEQ ID | SEQ ID | SEQ ID | SEQ ID | SEQ ID | SEQ ID |
| | NO: 85 | NO: 27 | NO: 28 | NO: 86 | NO: 30 | NO: 31 | NO: 32 |
| | | GFTFS | ISYDG | ARAPS | QSVLF | WAS | QQYYS |
| | | SYG | SNK | AFYFD | SSNNK | | TPPT |
| | | | | Y | NY | | |
| CD98hc11- | SEQ ID | SEQ ID | SEQ ID | SEQ ID | SEQ ID | SEQ ID | SEQ ID |
| H | NO: 87 | NO: 27 | NO: 28 | NO: 86 | NO: 30 | NO: 31 | NO: 32 |
| | | GFTFS | ISYDG | ARAPS | QSVLF | WAS | QQYYS |
| | | SYG | SNK | AFYFD | SSNNK | | TPPT |
| | | | | Y | NY | | |
| CD98hc11 | SEQ ID | SEQ ID | SEQ ID | SEQ ID | SEQ ID | SEQ ID | SEQ ID |
| sp | NO: 88 | NO: 27 | NO: 28 | NO: 86 | NO: 30 | NO: 31 | NO: 32 |
| | | GFTFS | ISYDG | ARAPS | QSVLF | WAS | QQYYS |
| | | SYG | SNK | AFYFD | SSNNK | | TPPT |
| | | | | Y | NY | | |
| CD98hc11 | SEQ ID | SEQ ID | SEQ ID | SEQ ID | SEQ ID | SEQ ID | SEQ ID |
| sp-H | NO: 89 | NO: 27 | NO: 28 | NO: 86 | NO: 30 | NO: 31 | NO: 32 |
| | | GFTFS | ISYDG | ARAPS | QSVLF | WAS | QQYYS |
| | | SYG | SNK | AFYFD | SSNNK | | TPPT |
| | | | | Y | NY | | |
| CD98hc | SEQ ID | SEQ ID | SEQ ID | SEQ ID | SEQ ID | SEQ ID | SEQ ID |
| 12 | NO: 90 | NO: 27 | NO: 28 | NO: 91 | NO: 30 | NO: 31 | NO: 32 |
| | | GFTFS | ISYDG | ARAPS | QSVLF | WAS | QQYYS |
| | | SYG | SNK | HFYFD | SSNNK | | TPPT |
| | | | | Y | NY | | |
| CD98hc | SEQ ID | SEQ ID | SEQ ID | SEQ ID | SEQ ID | SEQ ID | SEQ ID |
| 12-H | NO: 92 | NO: 27 | NO: 28 | NO: 91 | NO: 30 | NO: 31 | NO: 32 |
| | | GFTFS | ISYDG | ARAPS | QSVLF | WAS | QQYYS |
| | | SYG | SNK | HFYFD | SSNNK | | TPPT |
| | | | | Y | NY | | |
| CD98hc | SEQ ID | SEQ ID | SEQ ID | SEQ ID | SEQ ID | SEQ ID | SEQ ID |
| 12sp | NO: 93 | NO: 27 | NO: 28 | NO: 91 | NO: 30 | NO: 31 | NO: 32 |
| | | GFTFS | ISYDG | ARAPS | QSVLF | WAS | QQYYS |
| | | SYG | SNK | HFYFD | SSNNK | | TPPT |
| | | | | Y | NY | | |
| CD98hc | SEQ ID | SEQ ID | SEQ ID | SEQ ID | SEQ ID | SEQ ID | SEQ ID |
| 12sp-H | NO: 94 | NO: 27 | NO: 28 | NO: 91 | NO: 30 | NO: 31 | NO: 32 |
| | | GFTFS | ISYDG | ARAPS | QSVLF | WAS | QQYYS |
| | | SYG | SNK | HFYFD | SSNNK | | TPPT |
| | | | | Y | NY | | |

In certain embodiments, the VH3 and the VL3 comprise amino acid sequences at least 90%, at least 91%, at least 92%, at least 93%, at least 94%, at least 95%, at least 96%, at least 97%, at least 98%, or at least 99% identical to the respective VH and VL of a scFv identified as: SEQ ID NO: 19, SEQ ID NO: 26, SEQ ID NO: 33, SEQ ID NO: 34, SEQ ID NO: 35, SEQ ID NO: 36, SEQ ID NO: 43, SEQ ID NO: 49, SEQ ID NO: 55, SEQ ID NO: 59, SEQ ID NO: 66, SEQ ID NO: 71, SEQ ID NO: 78, SEQ ID NO: 85, SEQ ID NO: 87, SEQ ID NO: 88, SEQ ID NO: 89, SEQ ID NO: 90, SEQ ID NO: 92, SEQ ID NO: 93, SEQ ID NO: 94.

In certain embodiments, the VH3 and the VL3 comprise amino acid sequences identical to the respective VH and VL of a scFv identified as: SEQ ID NO: 19, SEQ ID NO: 26, SEQ ID NO: 33, SEQ ID NO: 34, SEQ ID NO: 35, SEQ ID NO: 36, SEQ ID NO: 43, SEQ ID NO: 49, SEQ ID NO: 55, SEQ ID NO: 59, SEQ ID NO: 66, SEQ ID NO: 71, SEQ ID NO: 78, SEQ ID NO: 85, SEQ ID NO: 87, SEQ ID NO: 88, SEQ ID NO: 89, SEQ ID NO: 90, SEQ ID NO: 92, SEQ ID NO: 93, SEQ ID NO: 94.

In certain embodiments, the multispecific antibody or antigen binding fragment thereof comprises (a) a first heavy chain (HC1) comprising the VH1, a first heavy chain constant region comprising a first Fc region (Fc1), and the scFv1, and (b) a first light chain (LC1) comprising the VL1 and a light chain constant region.

In certain embodiments, the multispecific antibody or antigen binding fragment thereof comprises (a) a second heavy chain (HC2) comprising the VH1 and a first heavy chain constant region comprising a second Fc region (Fc2), and (b) a second light chain (LC2) comprising the VL1 and a light chain constant region.

In certain embodiments, the multispecific antibody or antigen binding fragment thereof further comprises a second Fc region (Fc2).

In certain embodiments, the multispecific antibody or antigen binding fragment thereof comprises (a) a first heavy chain (HC1) comprising the VH2, and a first heavy chain constant region comprising a first Fc region (Fc1), (b) a first light chain (LC1) comprising the VL2 and a light chain constant region; and (c) a second heavy chain (HC2) comprising the scFv1, and a second heavy chain constant region comprising a second Fc region (Fc2).

In certain embodiments, the multispecific antibody or antigen binding fragment thereof comprises the first antigen binding region, the second antigen binding region and the third antigen binding region.

In certain embodiments, the multispecific antibody or antigen binding fragment thereof comprises (a) a first heavy chain (HC1) comprising the VH1, a first heavy chain constant region comprising a first Fc region (Fc1), and the scFv1, (b) a first light chain (LC1) comprising the VL1 and a light chain constant region; and (c) a second heavy chain (HC2) comprising a second single chain variable fragment (scFv2) and a first heavy chain constant region comprising a second Fc region (Fc2), wherein the scFv2 comprises the VH2 and VL2.

In certain embodiments, the multispecific antibody or antigen binding fragment thereof comprises (a) a first heavy chain (HC1) comprising the VH2, a first heavy chain constant region comprising a first Fc region (Fc1), and the scFv1, (b) a first light chain (LC1) comprising the VL2 and a light chain constant region; and (c) a second heavy chain (HC2) comprising a second single chain variable fragment (scFv2) and a first heavy chain constant region comprising a second Fc region (Fc2), wherein the scFv2 comprises the VH1 and VL1.

In certain embodiments, a scFv of the invention comprises a heavy chain variable region (H_{V}) covalently linked to a light chain variable region (L_{V}) via a flexible linker. The scFv can retain the specificity of the original immunoglobulin, despite removal of the constant regions and the introduction of the linker. In a scFv, the order of the domains can be either H_{V}-linker-L_{V}, or L_{V}-linker-H_{V}. The linker can be designed de novo or derived from known protein structure to provide a compatible length and conformational in bridging the variable domains of a scFv without serious steric interference. The linker can have 10 to about 25 amino acids in length. Preferably, the linker is a peptide linker spanning about 3.5 nm (35 Å) between the carboxy terminus of the variable domain and the amino terminus of the other domain without affecting the ability of the domains to fold and form an intact antigen-binding site (Huston et al., Methods in Enzymology, vol. 203, pp. 46-88, 1991, which is incorporated herein by reference in its entirety). The linker preferably comprises a hydrophilic sequence in order to avoid intercalation of the peptide within or between the variable domains throughout the protein folding (Argos, Journal of Molecular Biology, vol. 211, no. 4, pp. 943-958, 1990). For example, the linker can comprise Gly and Ser residues and/or together with the charged residues such as Glu, Thr and Lys interspersed to enhance the solubility. In one embodiment, the linker has the amino acid sequence of SEQ ID NO: 97 (GTEGKSSGSGSESKST). In another embodiment, the linker has the amino acid sequence of SEQ ID NO: 98 (GGSEGKSSGSGSESKSTGGS). Any other suitable linker can also be used in view of the present disclosure.

In certain embodiments, a scFv of the invention may be a stabilized scFv, herein referred to as stapled Fv (spFv). “Staple” refers to the scFv linker that contains one or two Cys residues which are capable of forming a disulfide bond with the anchor point Cys. As used herein, “VH Cysteine” or “VH Cys” refers to a Cys residue that resides in the VH framework. “VL Cysteine” or “VL Cys” refers to a Cys residue that resides in the VL framework. “Stabilized” refers to a scFv retaining comparable binding to hK2 when compared to a non-heated scFv sample are referred to as being thermostable. Stapled Fv are described in International publication No. WO2021/030657, which is incorporated by reference herein in its entirety.

In certain embodiments, an isolated single chain variable fragment (scFv) comprises a heavy chain variable region (VH), a linker (L) and a light chain variable region (VL), wherein the scFv comprises a first disulfide bond between a structurally conserved surface exposed VH cysteine (Cys) and a first L Cys; a second disulfide bond between a structurally conserved surface exposed VL Cys and a second L Cys; or the first disulfide bond between the structurally conserved surface exposed VH Cys and the first L Cys and the second disulfide bond between the structurally conserved surface exposed VL Cys and the second L Cys.

In certain embodiments, the scFv1 and scFv2 each independently comprise the first disulfide bond and the second disulfide bond.

In certain embodiments, the linker has the amino acid sequence of SEQ ID NO: 99 (GGGSGGSGGCPPCGGSGG).

In certain embodiments, the scFv further comprises a histidine at the N-terminus.

In certain embodiments, the scFv1 comprises an amino acid sequence having at least 90%, at least 91%, at least 92%, at least 93%, at least 94%, at least 95%, at least 96%, at least 97%, at least 98%, or at least 99% sequence identity to an amino acid sequence selected from the group consisting of: SEQ ID NO: 19, SEQ ID NO: 26, SEQ ID NO: 33, SEQ ID NO: 34, SEQ ID NO: 35, SEQ ID NO: 36, SEQ ID NO: 43, SEQ ID NO: 49, SEQ ID NO: 55, SEQ ID NO: 59, SEQ ID NO: 66, SEQ ID NO: 71, SEQ ID NO: 78, SEQ ID NO: 85, SEQ ID NO: 87, SEQ ID NO: 88, SEQ ID NO: 89, SEQ ID NO: 90, SEQ ID NO: 92, SEQ ID NO: 93, and SEQ ID NO: 94, preferably, the scFv1 comprises an amino acid sequence selected from the group consisting of SEQ ID NO: 19, SEQ ID NO: 26, SEQ ID NO: 33, SEQ ID NO: 34, SEQ ID NO: 35, SEQ ID NO: 36, SEQ ID NO: 43, SEQ ID NO: 49, SEQ ID NO: 55, SEQ ID NO: 59, SEQ ID NO: 66, SEQ ID NO: 71, SEQ ID NO: 78, SEQ ID NO: 85, SEQ ID NO: 87, SEQ ID NO: 88, SEQ ID NO: 89, SEQ ID NO: 90, SEQ ID NO: 92, SEQ ID NO: 93, and SEQ ID NO: 94.

In certain embodiments, the scFv2 comprises an amino acid sequence having at least 90%, at least 91%, at least 92%, at least 93%, at least 94%, at least 95%, at least 96%, at least 97%, at least 98%, or at least 99% s sequence identity to an amino acid sequence SEQ ID NO: 3 or 4, preferably, the scFv2 comprises an amino acid sequence of SEQ ID NO: 3 or 4.

### Antibody Variants

In some embodiments, amino acid sequence modification(s) of the antibodies that bind to HER2 described herein are contemplated. For example, it may be desirable to optimize the binding affinity and/or other biological properties of the antibody, including but not limited to specificity, thermostability, expression level, effector functions, glycosylation, reduced immunogenicity, or solubility. Thus, in addition to the antibodies that bind to HER2 described herein, it is contemplated that variants of the antibodies that bind to HER2 described herein can be prepared. For example, antibody variants can be prepared by introducing appropriate nucleotide changes into the encoding DNA, and/or by synthesis of the desired antibody or polypeptide. Those skilled in the art who appreciate that amino acid changes may alter post-translational processes of the antibody.

### Chemical Modifications

In some embodiments, the antibodies provided herein are chemically modified, for example, by the covalent attachment of any type of molecule to the antibody. The antibody derivatives may include antibodies that have been chemically modified, for example, by glycosylation, acetylation, pegylation, phosphorylation, amidation, derivatization by known protecting/blocking groups, proteolytic cleavage, linkage to a cellular ligand or other protein, or conjugation to one or more immunoglobulin domains (e.g., Fc or a portion of an Fc). Any of numerous chemical modifications may be carried out by known techniques, including, but not limited to, specific chemical cleavage, acetylation, formulation, metabolic synthesis of tunicamycin, etc. Additionally, the antibody may contain one or more non-classical amino acids.

In some embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

When the antibody provided herein is fused to an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in the binding molecules provided herein may be made in order to create variants with certain improved properties.

In other embodiments, when the antibody provided herein is fused to an Fc region, antibody variants provided herein may have a carbohydrate structure that lacks fucose attached (directly or indirectly) to said Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g., complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 and US 2004/0093621. Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87:614 (2004). Examples of cell lines capable of producing defucosylated antibodies include Lec13 CHO cells deficient in protein fucosylation (Ripka et al. Arch. Biochem. Biophys. 249:533-545 (1986); US Patent Application No. US 2003/0157108; and WO 2004/056312, and knockout cell lines, such as alpha-1,6-fucosyltransferase gene, FUT8, knockout CHO cells (see, e.g., Yamane-Ohnuki et al. Biotech. Bioeng. 87:614 (2004); Kanda, Y. et al., Biotechnol. Bioeng., 94(4): 680-688 (2006); and WO2003/085107).

The binding molecules comprising an antibody provided herein are further provided with bisected oligosaccharides, e.g., in which a biantennary oligosaccharide attached to the Fc region is bisected by GlcNAc. Such variants may have reduced fucosylation and/or improved ADCC function. Examples of such variants are described, e.g., in WO 2003/011878 (Jean-Mairet et al.); U.S. Pat. No. 6,602,684 (Umana et al.); and US 2005/0123546 (Umana et al.). Variants with at least one galactose residue in the oligosaccharide attached to the Fc region are also provided. Such variants may have improved CDC function. Such variants are described, e.g., in WO 1997/30087; WO 1998/58964; and WO 1999/22764.

In molecules that comprise the present antibody and an Fc region, one or more amino acid modifications may be introduced into the Fc region, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In some embodiments, the present application contemplates variants that possesses some but not all effector functions, which make it a desirable candidate for applications in which the half life of the binding molecule in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the binding molecule lacks FcγR binding (hence likely lacking ADCC activity), but retains FcRn binding ability. Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, CA; and CytoTox 96® non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half life determinations can also be performed using methods known in the art (see, e.g., Petkova, S. B. et al., Int'l. Immunol. 18(12):1759-1769 (2006)).

Binding molecules with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain variants with improved or diminished binding to FcRs are described. (See, e.g., U.S. Pat. No. 6,737,056; WO 2004/056312, and Shields et al., J. Biol. Chem. 9(2): 6591-6604 (2001).)

In some embodiments, a variant comprises an Fc region with one or more amino acid substitutions which improve ADCC, e.g., substitutions at positions 298, 333, and/or 334 of the Fc region (EU numbering of residues). In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164:4178-4184 (2000).

Binding molecules with increased half lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those molecules comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826). See also Duncan & Winter, Nature 322:738-40 (1988); U.S. Pat. Nos. 5,648,260; 5,624,821; and WO 94/29351 concerning other examples of Fc region variants.

In some embodiments, it may be desirable to create cysteine engineered antibodies, in which one or more residues of an antibody are substituted with cysteine residues. In some embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein.

### Substitutions, Deletions, or Insertions

Variations may be a substitution, deletion, or insertion of one or more codons encoding the antibody or polypeptide that results in a change in the amino acid sequence as compared with the original antibody or polypeptide. Sites of interest for substitutional mutagenesis include the CDRs and FRs.

Amino acid substitutions can be the result of replacing one amino acid with another amino acid having similar structural and/or chemical properties, such as the replacement of a leucine with a serine, e.g., conservative amino acid replacements. Standard techniques known to those of skill in the art can be used to introduce mutations in the nucleotide sequence encoding a molecule provided herein, including, for example, site-directed mutagenesis and PCR-mediated mutagenesis which results in amino acid substitutions. Insertions or deletions may optionally be in the range of about 1 to 5 amino acids. In certain embodiments, the substitution, deletion, or insertion includes fewer than 25 amino acid substitutions, fewer than 20 amino acid substitutions, fewer than 15 amino acid substitutions, fewer than 10 amino acid substitutions, fewer than 5 amino acid substitutions, fewer than 4 amino acid substitutions, fewer than 3 amino acid substitutions, or fewer than 2 amino acid substitutions relative to the original molecule. In a specific embodiment, the substitution is a conservative amino acid substitution made at one or more predicted non-essential amino acid residues. The variation allowed may be determined by systematically making insertions, deletions, or substitutions of amino acids in the sequence and testing the resulting variants for activity exhibited by the parental antibodies.

Amino acid sequence insertions include amino- and/or carboxyl-terminal fusions ranging in length from one residue to polypeptides containing multiple residues, as well as intrasequence insertions of single or multiple amino acid residues. Examples of terminal insertions include an antibody with an N-terminal methionyl residue.

Antibodies generated by conservative amino acid substitutions are included in the present disclosure. In a conservative amino acid substitution, an amino acid residue is replaced with an amino acid residue having a side chain with a similar charge. As described above, families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed and the activity of the protein can be determined. Conservative (e.g., within an amino acid group with similar properties and/or side chains) substitutions may be made, so as to maintain or not significantly change the properties. Exemplary substitutions are shown in Table 3 below.

TABLE 3
Amino Acid Substitutions

| Original | Exemplary |
|---|---|
| Residue | Substitutions |
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; |
| | Norleucine |
| Leu (L) | Norleucine; Ile; Val; Met; |
| | Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; |
| | Norleucine |

Amino acids may be grouped according to similarities in the properties of their side chains (see, e.g., Lehninger, Biochemistry 73-75 (2d ed. 1975)): (1) non-polar: Ala (A), Val (V), Leu (L), Ile (I), Pro (P), Phe (F), Trp (W), Met (M); (2) uncharged polar: Gly (G), Ser(S), Thr (T), Cys (C), Tyr (Y), Asn (N), Gln (Q); (3) acidic: Asp (D), Glu (E); and (4) basic: Lys (K), Arg (R), His (H). Alternatively, naturally occurring residues may be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; and (6) aromatic: Trp, Tyr, Phe. For example, any cysteine residue not involved in maintaining the proper conformation of the antibody also may be substituted, for example, with another amino acid, such as alanine or serine, to improve the oxidative stability of the molecule and to prevent aberrant crosslinking. Non-conservative substitutions will entail exchanging a member of one of these classes for another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more CDR residues are mutated and the variant antibodies displayed on phage and screened for a particular biological activity (e.g. binding affinity).

Alterations (e.g., substitutions) may be made in CDRs, e.g., to improve antibody affinity. Such alterations may be made in CDR “hotspots,” i.e., residues encoded by codons that undergo mutation at high frequency during the somatic maturation process (see, e.g., Chowdhury, Methods Mol. Biol. 207:179-196 (2008)), and/or SDRs (a-CDRs), with the resulting variant antibody or fragment thereof being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves CDR-directed approaches, in which several CDR residues (e.g., 4-6 residues at a time) are randomized. CDR residues involved in antigen binding may be specifically identified, e.g., using alanine scanning mutagenesis or modeling. More detailed description regarding affinity maturation is provided below.

In some embodiments, substitutions, insertions, or deletions may occur within one or more CDRs so long as such alterations do not substantially reduce the ability of the antibody to bind antigen. For example, conservative alterations (e.g., conservative substitutions as provided herein) that do not substantially reduce binding affinity may be made in CDRs. In some embodiments of the variant antibody sequences provided herein, each CDR either is unaltered, or contains no more than one, two or three amino acid substitutions.

A useful method for identification of residues or regions of an antibody that may be targeted for mutagenesis is called “alanine scanning mutagenesis” as described by Cunningham and Wells, Science, 244:1081-1085 (1989). In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

The variations can be made using methods known in the art such as oligonucleotide-mediated (site-directed) mutagenesis, alanine scanning, and PCR mutagenesis. Site-directed mutagenesis (see, e.g., Carter, Biochem J. 237:1-7 (1986); and Zoller et al., Nucl. Acids Res. 10:6487-500 (1982)), cassette mutagenesis (see, e.g., Wells et al., Gene 34:315-23 (1985)), or other known techniques can be performed on the cloned DNA to produce the antibody variant DNA.

### Fc Mutations

To facilitate the formation of a heterodimer between the two heavy chains, e.g., one with a fusion of the anti-HER2 antibody or antigen-binding fragment thereof and one without, or one containing the Fc for the anti-HER2 arm and one for the tissue target arm, heterodimeric mutations introduced into the Fc of the two heavy chains. Examples of such Fc mutations include, but are not limited to, the Zymework mutations (see, e.g., U.S. Pat. No. 10,457,742) and the “knob in hole” mutations (see, e.g., Ridgway et al., Protein Eng., 9(7): 617-621, 1996). Other heterodimer mutations can also be used in the present disclosure. In some embodiment, a modified CH3 as described herein is used to facilitate the formation of a heterodimer between the two heavy chains.

In a specific embodiment, each of the two heavy chains of the antibody comprises one or more heterodimeric mutation(s) or one or more knob and hole mutation(s). In a specific embodiment, the one or more heterodimeric mutation(s) is in the CH3 domain.

In certain embodiments, each of the two heavy chains of the multispecific antibody or antigen binding fragment thereof comprises a modified constant heavy chain 3 (CH3) domain as compared to a wild-type CH3 domain to facilitate the formation of a heterodimer between the two heavy chains. Any mutation that facilitates the formation of a heterodimer between the two heavy chains can be used. Preferably, the modified CH3 domain of the first heavy chain comprises amino acid modifications at positions T350, L351, F405, and Y407, and the modified CH3 domain of the second heavy chain comprises amino acid modifications at positions T350, T366, K392 and T394. Preferably, the amino acid modification at position T350 is T350V, T350I, T350L or T350M; the amino acid modification at position L351 is L351Y; the amino acid modification at position F405 is F405A, F405V, F405T or F405S; the amino acid modification at position Y407 is Y407V, Y407A or Y407I; the amino acid modification at position T366 is T366L, T366I, T366V or T366M, the amino acid modification at position K392 is K392F, K392L or K392M, and the amino acid modification at position T394 is T394W. More preferably, the modified heterodimeric CH3 domain of the first heavy chain comprises mutations T350V, L351Y, F405A and Y407V, and the modified heterodimeric CH3 domain of the second heavy chain comprises mutations T350V, T366L, K392L and T394W. The numbering of amino acid residues in the antibody throughout the specification is performed according to the EU index as described in Kabat et al., Sequences of Proteins of Immunological Interest, 5th Ed. Public Health Service, National Institutes of Health, Bethesda, Md. (1991), unless otherwise explicitly stated. In a specific embodiment, the CH3 domain of one heavy chain comprises mutations T350V, L351Y, F405A and Y407V, and the CH3 domain of the other heavy chain comprises mutations T350V, T366L, K392L and T394W.

In addition to the heterodimeric mutations, other mutations can also be introduced. In some embodiment, the Fc region of the antibody further comprises one or more mutations that alter (increase or diminish), preferably eliminate ADCC/CDC (such as the AAS mutations described herein), and/or one or more mutations that alter (increase or diminish), preferably increase, the binding of the antibody to FcRn (such as the YTE mutations described herein). In some embodiment, one or more cysteine residues in the antibody are substituted with other amino acids, such as serine.

In certain embodiments, the fragment crystallizable region (Fc region) of the multispecific antibody or antigen binding fragment thereof contains substitutions that alter (increase or diminish), preferably eliminate, effector function, such as antibody dependent cellular cytotoxicity (ADCC) and/or complement dependent cytotoxicity (CDC). Preferably, the Fc region of the multispecific antibody or antigen binding fragment thereof comprises one or more amino acid modifications that decrease or abolish the binding of the multispecific antibody or antigen binding fragment thereof to Fc gamma receptors (FcγR) and avoid effector function mediated toxicity. For example, the Fc region of the multispecific antibody or antigen binding fragment thereof can comprise one or more amino acid modifications at positions L234, L235, D270, N297, E318, K320, K322, P331, and P329, such as one, two or three mutations of L234A, L235A and D265S, wherein the numbering of amino acid residues is according to the EU index as set forth in Kabat.

In certain embodiments, the Fc region of the multispecific antibody or antigen binding fragment thereof contains substitutions that alter (increase or diminish), preferably increase, the binding of the multispecific antibody or antigen binding fragment thereof to neonatal Fc receptor (FcRn). Preferably the one or more mutations enhance the binding at an acidic pH, more preferably the Fc has the M252Y/S254T/T256E (YTE) mutations, wherein the numbering of amino acid residues is according to the EU index as set forth in Kabat.

In certain embodiments, the Fc region of the multispecific antibody or antigen binding fragment thereof contains the one or more mutations at positions M252Y, S254T, T256E, and wherein the numbering of amino acid residues is according to the EU index as set forth in Kabat. In certain embodiments, the Fc region of the multispecific antibody or antigen binding fragment thereof contains the one or more mutations at positions M252Y, S254T, T256E, L234A, L235A and D265S, and wherein the numbering of amino acid residues is according to the EU index as set forth in Kabat.

In certain embodiments, the multispecific antibody or antigen binding fragment thereof comprises a Fc domain not having amino acid modifications that reduce or eliminate the effector function.

Also provided is a multispecific antibody comprising a first heavy chain, a light chain and a second heavy chain each having the amino acid sequences at least 90%, at least 91%, at least 92%, at least 93%, at least 94%, at least 95%, at least 96%, at least 97%, at least 98%, or at least 99% identical to SEQ ID NO: 100, SEQ ID NO: 12, and SEQ ID NO: 101, respectively; or SEQ ID NO: 102, SEQ ID NO: 12, and SEQ ID NO: 103, respectively; wherein the first antigen-binding region is capable of binding specifically to a first epitope of HER2, the second antigen-binding region is capable of binding specifically to a second epitope of HER2, and the third antigen-binding region is capable of binding specifically to CD98. Preferably the first heavy chain, the light chain and the second heavy chain each comprises the amino acid sequences of: SEQ ID NO: 100, SEQ ID NO: 12, and SEQ ID NO: 101, respectively; or SEQ ID NO: 102, SEQ ID NO: 12, and SEQ ID NO: 103, respectively.

### Polynucleotides

In certain embodiments, the disclosure provides polynucleotides that encode the present antibodies that bind to HER2 and fusion proteins comprising the antibodies that bind to HER2 described herein. The polynucleotides of the disclosure can be in the form of RNA or in the form of DNA. DNA includes cDNA, genomic DNA, and synthetic DNA; and can be double-stranded or single-stranded, and if single stranded can be the coding strand or non-coding (anti-sense) strand. In some embodiments, the polynucleotide is in the form of cDNA. In some embodiments, the polynucleotide is a synthetic polynucleotide.

The present disclosure further relates to variants of the polynucleotides described herein, wherein the variant encodes, for example, fragments, analogs, and/or derivatives of the antibody that binds HER2 of the disclosure. In certain embodiments, the present disclosure provides a polynucleotide comprising a polynucleotide having a nucleotide sequence at least about 75% identical, at least about 80% identical, at least about 85% identical, at least about 90% identical, at least about 95% identical, and in some embodiments, at least about 96%, 97%, 98% or 99% identical to a polynucleotide encoding the antibody that binds HER2 of the disclosure. As used herein, the phrase “a polynucleotide having a nucleotide sequence at least, for example, 95% “identical” to a reference nucleotide sequence” is intended to mean that the nucleotide sequence of the polynucleotide is identical to the reference sequence except that the polynucleotide sequence can include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence can be deleted or substituted with another nucleotide, or a number of nucleotides up to 5% of the total nucleotides in the reference sequence can be inserted into the reference sequence. These mutations of the reference sequence can occur at the 5′ or 3′ terminal positions of the reference nucleotide sequence or anywhere between those terminal positions, interspersed either individually among nucleotides in the reference sequence or in one or more contiguous groups within the reference sequence.

The polynucleotide variants can contain alterations in the coding regions, non-coding regions, or both. In some embodiments, a polynucleotide variant contains alterations which produce silent substitutions, additions, or deletions, but does not alter the properties or activities of the encoded polypeptide. In some embodiments, a polynucleotide variant comprises silent substitutions that results in no change to the amino acid sequence of the polypeptide (due to the degeneracy of the genetic code). Polynucleotide variants can be produced for a variety of reasons, for example, to optimize codon expression for a particular host (i.e., change codons in the human mRNA to those preferred by a bacterial host such as E. coli). In some embodiments, a polynucleotide variant comprises at least one silent mutation in a non-coding or a coding region of the sequence.

In some embodiments, a polynucleotide variant is produced to modulate or alter expression (or expression levels) of the encoded polypeptide. In some embodiments, a polynucleotide variant is produced to increase expression of the encoded polypeptide. In some embodiments, a polynucleotide variant is produced to decrease expression of the encoded polypeptide. In some embodiments, a polynucleotide variant has increased expression of the encoded polypeptide as compared to a parental polynucleotide sequence. In some embodiments, a polynucleotide variant has decreased expression of the encoded polypeptide as compared to a parental polynucleotide sequence.

Also provided are vectors comprising the nucleic acid molecules described herein. In an embodiment, the nucleic acid molecules can be incorporated into a recombinant expression vector. The present disclosure provides recombinant expression vectors comprising any of the nucleic acids of the disclosure. As used herein, the term “recombinant expression vector” means a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell. The vectors described herein are not naturally-occurring as a whole; however, parts of the vectors can be naturally-occurring. The described recombinant expression vectors can comprise any type of nucleotides, including, but not limited to DNA and RNA, which can be single-stranded or double-stranded, synthesized or obtained in part from natural sources, and which can contain natural, non-natural or altered nucleotides. The recombinant expression vectors can comprise naturally-occurring or non-naturally-occurring internucleotide linkages, or both types of linkages. The non-naturally occurring or altered nucleotides or internucleotide linkages do not hinder the transcription or replication of the vector.

In an embodiment, the recombinant expression vector of the disclosure can be any suitable recombinant expression vector, and can be used to transform or transfect any suitable host. Suitable vectors include those designed for propagation and expansion or for expression or both, such as plasmids and viruses. The vector can be selected from the group consisting of the pUC series (Fermentas Life Sciences, Glen Burnie, Md.), the pBluescript series (Stratagene, LaJolla, Calif.), the pET series (Novagen, Madison, Wis.), the pGEX series (Pharmacia Biotech, Uppsala, Sweden), and the pEX series (Clontech, Palo Alto, Calif.). Bacteriophage vectors, such as λGT10, λGT11, λEMBL4, and λNM1149, λZapII (Stratagene) can be used. Examples of plant expression vectors include pBI01, pBI01.2, pBI121, pBI101.3, and pBIN19 (Clontech). Examples of animal expression vectors include pEUK-Cl, pMAM, and pMAMneo (Clontech). The recombinant expression vector may be a viral vector, e.g., a retroviral vector, e.g., a gamma retroviral vector.

In an embodiment, the recombinant expression vectors are prepared using standard recombinant DNA techniques described in, for example, Sambrook et al., supra, and Ausubel et al., supra. Constructs of expression vectors, which are circular or linear, can be prepared to contain a replication system functional in a prokaryotic or eukaryotic host cell. Replication systems can be derived, e.g., from ColE1, SV40, 2μ plasmid, λ, bovine papilloma virus, and the like.

The recombinant expression vector may comprise regulatory sequences, such as transcription and translation initiation and termination codons, which are specific to the type of host (e.g., bacterium, plant, fungus, or animal) into which the vector is to be introduced, as appropriate, and taking into consideration whether the vector is DNA- or RNA-based.

The recombinant expression vector can include one or more marker genes, which allow for selection of transformed or transfected hosts. Marker genes include biocide resistance, e.g., resistance to antibiotics, heavy metals, etc., complementation in an auxotrophic host to provide prototrophy, and the like. Suitable marker genes for the described expression vectors include, for instance, neomycin/G418 resistance genes, histidinol×resistance genes, histidinol resistance genes, tetracycline resistance genes, and ampicillin resistance genes.

The recombinant expression vector can comprise a native or normative promoter operably linked to the nucleotide sequence of the disclosure. The selection of promoters, e.g., strong, weak, tissue-specific, inducible and developmental-specific, is within the ordinary skill of the artisan. Similarly, the combining of a nucleotide sequence with a promoter is also within the skill of the artisan. The promoter can be a non-viral promoter or a viral promoter, e.g., a cytomegalovirus (CMV) promoter, an RSV promoter, an SV40 promoter, or a promoter found in the long-terminal repeat of the murine stem cell virus.

The recombinant expression vectors can be designed for either transient expression, for stable expression, or for both. Also, the recombinant expression vectors can be made for constitutive expression or for inducible expression.

Further, the recombinant expression vectors can be made to include a suicide gene. As used herein, the term “suicide gene” refers to a gene that causes the cell expressing the suicide gene to die. The suicide gene can be a gene that confers sensitivity to an agent, e.g., a drug, upon the cell in which the gene is expressed, and causes the cell to die when the cell is contacted with or exposed to the agent. Suicide genes are known in the art and include, for example, the Herpes Simplex Virus (HSV) thymidine kinase (TK) gene, cytosine deaminase, purine nucleoside phosphorylase, and nitroreductase.

In certain embodiments, a polynucleotide is isolated. In certain embodiments, a polynucleotide is substantially pure.

Also provided are host cells comprising the nucleic acid molecules described herein. The host cell may be any cell that contains a heterologous nucleic acid. The heterologous nucleic acid can be a vector (e.g., an expression vector). For example, a host cell can be a cell from any organism that is selected, modified, transformed, grown, used or manipulated in any way, for the production of a substance by the cell, for example the expression by the cell of a gene, a DNA or RNA sequence, a protein or an enzyme. An appropriate host may be determined. For example, the host cell may be selected based on the vector backbone and the desired result. By way of example, a plasmid or cosmid can be introduced into a prokaryote host cell for replication of several types of vectors. Bacterial cells such as, but not limited to DH5α, JM109, and KCB, SURE® Competent Cells, and SOLOPACK Gold Cells, can be used as host cells for vector replication and/or expression. Additionally, bacterial cells such as E. coli LE392 could be used as host cells for phage viruses. Eukaryotic cells that can be used as host cells include, but are not limited to yeast (e.g., YPH499, YPH500 and YPH501), insects and mammals. Examples of mammalian eukaryotic host cells for replication and/or expression of a vector include, but are not limited to, HeLa, NIH3T3, Jurkat, 293, COS, Saos, PC12, SP2/0 (American Type Culture Collection (ATCC), Manassas, VA, CRL-1581), NS0 (European Collection of Cell Cultures (ECACC), Salisbury, Wiltshire, UK, ECACC No. 85110503), FO (ATCC CRL-1646) and Ag653 (ATCC CRL-1580) murine cell lines. An exemplary human myeloma cell line is U266 (ATCC CRL-TIB-196). Other useful cell lines include those derived from Chinese Hamster Ovary (CHO) cells such as CHO-KISV (Lonza Biologics, Walkersville, MD), CHO-K1 (ATCC CRL-61) or DG44.

### Preparation of Antibodies and Method of Making

Methods of preparing antibodies have been described. See, e.g., Els Pardon et al, Nature Protocol, 9(3): 674 (2014). Antibodies (such as scFv fragments) may be obtained using methods known in the art such as by immunizing a Camelid species (such as camel or llama) and obtaining hybridomas therefrom, or by cloning a library of antibodies using molecular biology techniques known in the art and subsequent selection by ELISA with individual clones of unselected libraries or by using phage display.

Antibodies provided herein may be produced by culturing cells transformed or transfected with a vector containing an antibody-encoding nucleic acids. Polynucleotide sequences encoding polypeptide components of the antibody of the present disclosure can be obtained using standard recombinant techniques. Desired polynucleotide sequences may be isolated and sequenced from antibody producing cells such as hybridomas cells or B cells. Alternatively, polynucleotides can be synthesized using nucleotide synthesizer or PCR techniques. Once obtained, sequences encoding the polypeptides are inserted into a recombinant vector capable of replicating and expressing heterologous polynucleotides in host cells. Many vectors that are available and known in the art can be used for the purpose of the present disclosure. Selection of an appropriate vector will depend mainly on the size of the nucleic acids to be inserted into the vector and the particular host cell to be transformed with the vector. Host cells suitable for expressing antibodies of the present disclosure include prokaryotes such as Archaebacteria and Eubacteria, including Gram-negative or Gram-positive organisms, eukaryotic microbes such as filamentous fungi or yeast, invertebrate cells such as insect or plant cells, and vertebrate cells such as mammalian host cell lines. Host cells are transformed with the above-described expression vectors and cultured in conventional nutrient media modified as appropriate for inducing promoters, selecting transformants, or amplifying the genes encoding the desired sequences. Antibodies produced by the host cells are purified using standard protein purification methods as known in the art.

Methods for antibody production including vector construction, expression, and purification are further described in Plückthun et al., Antibody Engineering: Producing antibodies in Escherichia coli: From PCR to fermentation 203-52 (McCafferty et al. eds., 1996); Kwong and Rader, E. coli Expression and Purification of Fab Antibody Fragments, in Current Protocols in Protein Science (2009); Tachibana and Takekoshi, Production of Antibody Fab Fragments in Escherichia coli, in Antibody Expression and Production (Al-Rubeai ed., 2011); and Therapeutic Monoclonal Antibodies: From Bench to Clinic (An ed., 2009).

It is, of course, contemplated that alternative methods, which are well known in the art, may be employed to prepare anti-HER2 antibodies. For instance, the appropriate amino acid sequence, or portions thereof, may be produced by direct peptide synthesis using solid-phase techniques (see, e.g., Stewart et al., Solid-Phase Peptide Synthesis (1969); and Merrifield, J. Am. Chem. Soc. 85:2149-54 (1963)). In vitro protein synthesis may be performed using manual techniques or by automation. Various portions of the anti-HER2 antibody may be chemically synthesized separately and combined using chemical or enzymatic methods to produce the desired anti-HER2 antibody. Alternatively, antibodies may be purified from cells or bodily fluids, such as milk, of a transgenic animal engineered to express the antibody, as disclosed, for example, in U.S. Pat. Nos. 5,545,807 and 5,827,690.

### Pharmaceutical Compositions

In one aspect, the present disclosure further provides pharmaceutical compositions comprising the multispecific antibody or antigen binding fragment thereof of the present disclosure. In some embodiments, a pharmaceutical composition comprises therapeutically effective amount of the multispecific antibody or antigen binding fragment thereof provided herein and a pharmaceutically acceptable excipient.

Pharmaceutical compositions comprising the multispecific antibody or antigen binding fragment thereof are prepared for storage by mixing the fusion protein having the desired degree of purity with optional physiologically acceptable excipients (see, e.g., Remington, Remington's Pharmaceutical Sciences (18th ed. 1980)) in the form of aqueous solutions or lyophilized or other dried forms.

The multispecific antibody or antigen binding fragment thereof of the present disclosure may be formulated in any suitable form for delivery to a target cell/tissue, e.g., as microcapsules or macroemulsions (Remington, supra; Park et al., 2005, Molecules 10:146-61; Malik et al., 2007, Curr. Drug. Deliv. 4:141-51), as sustained release formulations (Putney and Burke, 1998, Nature Biotechnol. 16:153-57), or in liposomes (Maclean et al., 1997, Int. J. Oncol. 11:325-32; Kontermann, 2006, Curr. Opin. Mol. Ther. 8:39-45).

An antibody or antigen binding fragment thereof provided herein can also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles, and nanocapsules) or in macroemulsions. Such techniques are disclosed, for example, in Remington, supra.

Various compositions and delivery systems are known and can be used with an antibody or antigen binding fragment thereof as described herein, including, but not limited to, encapsulation in liposomes, microparticles, microcapsules, recombinant cells capable of expressing the multispecific antibody or antigen binding fragment thereof, receptor-mediated endocytosis (see, e.g., Wu and Wu, 1987, J. Biol. Chem. 262:4429-32), construction of a nucleic acid as part of a retroviral or other vector, etc. In another embodiment, a composition can be provided as a controlled release or sustained release system. In one embodiment, a pump may be used to achieve controlled or sustained release (see, e.g., Langer, supra; Sefton, 1987, Crit. Ref. Biomed. Eng. 14:201-40; Buchwald et al., 1980, Surgery 88:507-16; and Saudek et al., 1989, N. Engl. J. Med. 321:569-74). In another embodiment, polymeric materials can be used to achieve controlled or sustained release of a prophylactic or therapeutic agent (e.g., an antibody or antigen binding fragment thereof as described herein) or a composition provided herein (see, e.g., Medical Applications of Controlled Release (Langer and Wise eds., 1974); Controlled Drug Bioavailability, Drug Product Design and Performance (Smolen and Ball eds., 1984); Ranger and Peppas, 1983, J. Macromol. Sci. Rev. Macromol. Chem. 23:61-126; Levy et al., 1985, Science 228:190-92; During et al., 1989, Ann. Neurol. 25:351-56; Howard et al., 1989, J. Neurosurg. 71:105-12; U.S. Pat. Nos. 5,679,377; 5,916,597; 5,912,015; 5,989,463; and 5,128,326; PCT Publication Nos. WO 99/15154 and WO 99/20253). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In one embodiment, the polymer used in a sustained release formulation is inert, free of leachable impurities, stable on storage, sterile, and biodegradable.

In yet another embodiment, a controlled or sustained release system can be placed in proximity of a particular target tissue, for example, the nasal passages or lungs, thus requiring only a fraction of the systemic dose (see, e.g., Goodson, Medical Applications of Controlled Release Vol. 2, 115-38 (1984)). Controlled release systems are discussed, for example, by Langer, 1990, Science 249:1527-33. Any technique known to one of skill in the art can be used to produce sustained release formulations comprising one or more antibody or antigen binding fragment thereof as described herein (see, e.g., U.S. Pat. No. 4,526,938, PCT publication Nos. WO 91/05548 and WO 96/20698, Ning et al., 1996, Radiotherapy & Oncology 39:179-89; Song et al., 1995, PDA J. of Pharma. Sci. & Tech. 50:372-97; Cleek et al., 1997, Pro. Int'l. Symp. Control. Rel. Bioact. Mater. 24:853-54; and Lam et al., 1997, Proc. Int'l. Symp. Control Rel. Bioact. Mater. 24:759-60).

### Methods of Using the Antibodies

In one aspect, provided herein is a method of treating or detecting a disorder, in a subject in need thereof, comprising administering to the subject the multispecific antibody or antibody fragment thereof provided herein.

In one aspect, provided herein is a method of delivering a therapeutic or diagnostic agent to a particular tissue of a subject in need thereof, comprising administering to the subject the multispecific antibody or antibody fragment provided herein.

In one aspect, provided herein is a method of inducing antibody dependent phagocytosis (ADP) without stimulating secretion of a pro-inflammatory cytokine in a subject in need thereof, comprising administering to the subject the multispecific antibody or antigen binding fragment provided herein.

In one aspect, provided herein is a method of reducing or eliminating the effector function.

In one aspect, provided herein is a method of attenuating an activity of HER2 on a cell, comprising exposing the cell to an effective amount of the multispecific antibody or antigen binding fragment thereof provided herein.

In another aspect, provided herein is a method of treating a disease or disorder in a subject comprising administering to the subject an effective amount of an antibody or antigen binding fragment thereof provided herein. In one embodiment, the disease or disorder is an HER2-mediated disease or disorder. Also provided herein is a method of treatment of a disease or disorder, wherein the subject is administered one or more therapeutic agents in combination with the antibody or antigen-binding fragment thereof or provided herein.

The disclosure also relates to methods of using the antibodies provided herein to inhibit, i.e. antagonize, function of HER2 in order to inhibit HER2 activation resulting in the treatment of a pathological disorder.

The pathological disorder may be cancer. Examples of cancer to be treated herein include, but are not limited to, carcinoma, lymphoma, blastoma, sarcoma, and leukemia or lymphoid malignancies. More particular examples of such cancers include squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

In certain embodiments, the disease or disorder is brain metastasis.

In another aspect, provided herein is the use of the multispecific antibody or antigen binding fragment thereof provided herein in the manufacture of a medicament for treating a disease or disorder in a subject.

In another aspect, provided herein is the use of a pharmaceutical composition provided herein in the manufacture of a medicament for treating a disease or disorder in a subject.

In another aspect, provided herein is the use of the multispecific antibody or antigen binding fragment thereof provided herein in the manufacture of a medicament, wherein the medicament is for use in a method for detecting the presence of a HER2 in a biological sample, the method comprising contacting the biological sample with the antibody under conditions permissive for binding of the antibody to the HER2 protein, and detecting whether a complex is formed between the antibody and the HER2 protein.

In other aspects, the antibodies and fragments thereof of the present disclosure are useful for detecting the presence of a HER2 in a biological sample. The term “detecting” as used herein encompasses quantitative or qualitative detection. In certain embodiments, a biological sample comprises bodily fluid, a cell, or a tissue. Diagnostic assays and methods are described in more detail below.

### Methods of Administration and Dosing

In a specific embodiment, provided herein is a composition for use in the prevention and/or treatment of a disease or condition comprising an antibody or antigen binding fragment thereof provided herein. In one embodiment, provided herein is a composition for use in the prevention of a disease or condition, wherein the composition comprises an antibody or antigen binding fragment thereof provided herein. In one embodiment, provided herein is a composition for use in the treatment of a disease or condition, wherein the composition comprises an antibody or antigen binding fragment thereof provided herein. In some embodiments, the disease or condition is an HER2-mediated disease. In some embodiments, the disease or disorder is associated with HER2. In some embodiments, the disease or disorder is cancer. Examples of cancer to be treated herein include, but are not limited to, carcinoma, lymphoma, blastoma, sarcoma, and leukemia or lymphoid malignancies. More particular examples of such cancers include squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer. In certain embodiments, the subject is a subject in need thereof. In some embodiments, the subject has the disease or condition. In other embodiments, the subject is at risk of having the disease or condition. In some embodiments, the administration results in the prevention, management, treatment or amelioration of the disease or condition.

In one embodiment, provided herein is a composition for use in the prevention and/or treatment of a symptom of a disease or condition, wherein the composition comprises an antibody or antigen binding fragment thereof provided herein. In one embodiment, provided herein is a composition for use in the prevention of a symptom of a disease or condition, wherein the composition comprises an antibody or antigen binding fragment thereof provided herein. In one embodiment, provided herein is a composition for use in the treatment of a symptom of a disease or condition, wherein the composition comprises an antibody or antigen binding fragment thereof provided herein. In some embodiments, the disease or condition is an HER2-mediated and/or HER2 mediated disease. In some embodiments, the disease or disorder is associated with HER2. In some embodiments, the disease or disorder is cancer. Examples of cancer to be treated herein include, but are not limited to, carcinoma, lymphoma, blastoma, sarcoma, and leukemia or lymphoid malignancies. More particular examples of such cancers include squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer. In certain embodiments, the subject is a subject in need thereof. In some embodiments, the subject has the disease or condition. In other embodiments, the subject is at risk of having the disease or condition. In some embodiments, the administration results in the prevention or treatment of the symptom of the disease or condition.

In another embodiment, provided herein is a method of preventing and/or treating a disease or condition in a subject, comprising administering an effective amount of an antibody or antigen binding fragment thereof provided herein. In one embodiment, provided herein is a method of preventing a disease or condition in a subject, comprising administering an effective amount of an antibody or antigen binding fragment thereof provided herein. In one embodiment, provided herein is a method of treating a disease or condition in a subject, comprising administering an effective amount of an antibody or antigen binding fragment thereof provided herein. In some embodiments, the disease or condition is an HER2-mediated and/or HER2 mediated disease. In some embodiments, the disease or disorder is associated with HER2. In some embodiments, the disease or disorder is cancer. Examples of cancer to be treated herein include, but are not limited to, carcinoma, lymphoma, blastoma, sarcoma, and leukemia or lymphoid malignancies. More particular examples of such cancers include squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer. In certain embodiments, the subject is a subject in need thereof. In some embodiments, the subject has the disease or condition. In other embodiments, the subject is at risk of having the disease or condition. In some embodiments, the administration results in the prevention or treatment of the disease or condition.

In another embodiment, provided herein is a method of preventing and/or treating a symptom of a disease or condition in a subject, comprising administering an effective amount of an antibody or antigen binding fragment thereof provided herein. In one embodiment, provided herein is a method of preventing a symptom of a disease or condition in a subject, comprising administering an effective amount of an antibody or antigen binding fragment thereof provided herein. In one embodiment, provided herein is a method of treating a symptom of a disease or condition in a subject, comprising administering an effective amount of an antibody or antigen binding fragment thereof provided herein. In some embodiments, the disease or disorder is an HER2-mediated and/or HER2-associated disease or disorder. In some embodiments, the disease or disorder is associated with HER2. In some embodiments, the disease or disorder is cancer. Examples of cancer to be treated herein include, but are not limited to, carcinoma, lymphoma, blastoma, sarcoma, and leukemia or lymphoid malignancies. More particular examples of such cancers include squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer. In certain embodiments, the subject is a subject in need thereof. In some embodiments, the subject has the disease or condition. In other embodiments, the subject is at risk of having the disease or condition. In some embodiments, the administration results in the prevention or treatment of the symptom of the disease or condition.

Also provided herein are methods of preventing and/or treating a disease or condition by administrating to a subject of an effective amount of an antibody or antigen binding fragment thereof provided herein, or pharmaceutical composition comprising an antibody or antigen binding fragment thereof provided herein. In one aspect, the multispecific antibody or antigen binding fragment thereof is substantially purified (i.e., substantially free from substances that limit its effect or produce undesired side-effects). The subject administered a therapy can be a mammal such as non-primate (e.g., cows, pigs, horses, cats, dogs, rats etc.) or a primate (e.g., a monkey, such as a cynomolgus macaque monkey, or a human). In a one embodiment, the subject is a human. In another embodiment, the subject is a human with a disease or condition.

Various delivery systems are known and can be used to administer a prophylactic or therapeutic agent (e.g., an antibody or antigen binding fragment thereof provided herein), including, but not limited to, encapsulation in liposomes, microparticles, microcapsules, recombinant cells capable of expressing the multispecific antibody or antigen binding fragment thereof, receptor-mediated endocytosis (see, e.g., Wu and Wu, J. Biol. Chem. 262:4429-4432 (1987)), construction of a nucleic acid as part of a retroviral or other vector, etc. Methods of administering a prophylactic or therapeutic agent (e.g., an antibody or antigen binding fragment thereof provided herein), or pharmaceutical composition include, but are not limited to, parenteral administration (e.g., intradermal, intramuscular, intraperitoneal, intravenous and subcutaneous), epidural, and mucosal (e.g., intranasal and oral routes). In a specific embodiment, a prophylactic or therapeutic agent (e.g., an antibody or antigen binding fragment thereof provided herein), or a pharmaceutical composition is administered intranasally, intramuscularly, intravenously, or subcutaneously. The prophylactic or therapeutic agents, or compositions may be administered by any convenient route, for example by infusion or bolus injection, by absorption through epithelial or mucocutaneous linings (e.g., oral mucosa, intranasal mucosa, rectal and intestinal mucosa, etc.) and may be administered together with other biologically active agents. Administration can be systemic or local. In addition, pulmonary administration can also be employed, e.g., by use of an inhaler or nebulizer, and formulation with an aerosolizing agent. See, e.g., U.S. Pat. Nos. 6,019,968, 5,985,320, 5,985,309, 5,934,272, 5,874,064, 5,855,913, 5,290,540, and 4,880,078; and PCT Publication Nos. WO 92/19244, WO 97/32572, WO 97/44013, WO 98/31346, and WO 99/66903, each of which is incorporated herein by reference their entirety.

In a specific embodiment, it may be desirable to administer a prophylactic or therapeutic agent, or a pharmaceutical composition provided herein locally to the area in need of treatment. This may be achieved by, for example, and not by way of limitation, local infusion, by topical administration (e.g., by intranasal spray), by injection, or by means of an implant, said implant being of a porous, non-porous, or gelatinous material, including membranes, such as sialastic membranes, or fibers. In some embodiments, when administering an antibody or antigen binding fragment thereof provided herein, care must be taken to use materials to which the antibody or antigen binding fragment thereof does not absorb.

In another embodiment, a prophylactic or therapeutic agent, or a composition provided herein can be delivered in a vesicle, in particular a liposome (see Langer, 1990, Science 249:1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.).

In another embodiment, a prophylactic or therapeutic agent, or a composition provided herein can be delivered in a controlled release or sustained release system. In one embodiment, a pump may be used to achieve controlled or sustained release (see Langer, supra; Sefton, 1987, CRC Crit. Ref. Biomed. Eng. 14:20; Buchwald et al., 1980, Surgery 88:507; Saudek et al., 1989, N. Engl. J. Med. 321:574). In another embodiment, polymeric materials can be used to achieve controlled or sustained release of a prophylactic or therapeutic agent (e.g., an antibody provided herein) or a composition provided herein (see e.g., Medical Applications of Controlled Release, Langer and Wise (eds.), CRC Pres., Boca Raton, Florida (1974); Controlled Drug Bioavailability, Drug Product Design and Performance, Smolen and Ball (eds.), Wiley, New York (1984); Ranger and Peppas, 1983, J., Macromol. Sci. Rev. Macromol. Chem. 23:61; see also Levy et al., 1985, Science 228:190; During et al., 1989, Ann. Neurol. 25:351; Howard et al., 1989, J. Neurosurg. 7 1:105); U.S. Pat. Nos. 5,679,377; 5,916,597; 5,912,015; 5,989,463; 5,128,326; PCT Publication No. WO 99/15154; and PCT Publication No. WO 99/20253. Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In an embodiment, the polymer used in a sustained release formulation is inert, free of leachable impurities, stable on storage, sterile, and biodegradable. In yet another embodiment, a controlled or sustained release system can be placed in proximity of the therapeutic target, i.e., the nasal passages or lungs, thus requiring only a fraction of the systemic dose (see, e.g., Goodson, in Medical Applications of Controlled Release, supra, vol. 2, pp. 115-138 (1984)). Controlled release systems are discussed in the review by Langer (1990, Science 249:1527-1533). Any technique known to one of skill in the art can be used to produce sustained release formulations comprising one or more antibody or antigen binding fragment thereof provided herein. See, e.g., U.S. Pat. No. 4,526,938, PCT publication WO 91/05548, PCT publication WO 96/20698, Ning et al., 1996, “Intratumoral Radioimmunotherapy of a Human Colon Cancer Xenograft Using a Sustained-Release Gel,” Radiotherapy & Oncology 39:179-189, Song et al., 1995, “Antibody Mediated Lung Targeting of Long-Circulating Emulsions,” PDA Journal of Pharmaceutical Science & Technology 50:372-397, Cleek et al., 1997, “Biodegradable Polymeric Carriers for a bFGF Antibody for Cardiovascular Application,” Pro. Int'l. Symp. Control. Rel. Bioact. Mater. 24:853-854, and Lam et al., 1997, “Microencapsulation of Recombinant Humanized Monoclonal Antibody for Local Delivery,” Proc. Int'l. Symp. Control Rel. Bioact. Mater. 24:759-760, each of which is incorporated herein by reference in their entirety.

In a specific embodiment, where the composition provided herein is a nucleic acid encoding a prophylactic or therapeutic agent (e.g., an antibody or antigen binding fragment thereof provided herein), the nucleic acid can be administered in vivo to promote expression of its encoded prophylactic or therapeutic agent, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell surface receptors or transfecting agents, or by administering it in linkage to a homeobox-like peptide which is known to enter the nucleus (see, e.g., Joliot et al., 1991, Proc. Natl. Acad. Sci. USA 88:1864-1868), etc. Alternatively, a nucleic acid can be introduced intracellularly and incorporated within host cell DNA for expression by homologous recombination.

In a specific embodiment, a composition provided herein comprises one, two or more antibodies or antigen binding fragments or thereof provided herein. In another embodiment, a composition provided herein comprises one, two or more antibodies or antigen binding fragments thereof provided herein and a prophylactic or therapeutic agent other than an antibody or antigen binding fragment thereof provided herein. In one embodiment, the agents are known to be useful for or have been or are currently used for the prevention, management, treatment and/or amelioration of a disease or condition. In addition to prophylactic or therapeutic agents, the compositions provided herein may also comprise an excipient.

The compositions provided herein include bulk drug compositions useful in the manufacture of pharmaceutical compositions (e.g., compositions that are suitable for administration to a subject or patient) that can be used in the preparation of unit dosage forms. In an embodiment, a composition provided herein is a pharmaceutical composition. Such compositions comprise a prophylactically or therapeutically effective amount of one or more prophylactic or therapeutic agents (e.g., an antibody or antigen binding fragment thereof provided herein or other prophylactic or therapeutic agent), and a pharmaceutically acceptable excipient. The pharmaceutical compositions can be formulated to be suitable for the route of administration to a subject.

In a specific embodiment, the term “excipient” can also refer to a diluent, adjuvant (e.g., Freunds' adjuvant (complete or incomplete) or vehicle. Pharmaceutical excipients can be sterile liquids, such as water and oils, including those of petroleum, animal, vegetable or synthetic origin, such as peanut oil, soybean oil, mineral oil, sesame oil and the like. Water is an exemplary excipient when the pharmaceutical composition is administered intravenously. Saline solutions and aqueous dextrose and glycerol solutions can also be employed as liquid excipients, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulation can include standard excipients such as pharmaceutical grades of mannitol, lactose, starch, magnesium stearate, sodium saccharine, cellulose, magnesium carbonate, etc. Examples of suitable pharmaceutical excipients are described in Remington's Pharmaceutical Sciences (1990) Mack Publishing Co., Easton, PA. Such compositions will contain a prophylactically or therapeutically effective amount of the multispecific antibody or antigen binding fragment thereof provided herein, such as in purified form, together with a suitable amount of excipient so as to provide the form for proper administration to the patient. The formulation should suit the mode of administration.

In an embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Such compositions, however, may be administered by a route other than intravenous.

Generally, the ingredients of compositions provided herein are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

An antibody or antigen binding fragment thereof provided herein can be packaged in a hermetically sealed container such as an ampoule or sachette indicating the quantity of antibody. In one embodiment, the multispecific antibody or antigen binding fragment thereof is supplied as a dry sterilized lyophilized powder or water free concentrate in a hermetically sealed container and can be reconstituted, e.g., with water or saline to the appropriate concentration for administration to a subject. The lyophilized antibody or antigen binding fragment thereof can be stored at between 2 and 8° C. in its original container and the multispecific antibody or antigen binding fragment thereof can be administered within 12 hours, such as within 6 hours, within 5 hours, within 3 hours, or within 1 hour after being reconstituted. In an alternative embodiment, an antibody or antigen binding fragment thereof provided herein is supplied in liquid form in a hermetically sealed container indicating the quantity and concentration of the antibody.

The compositions provided herein can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The amount of a prophylactic or therapeutic agent (e.g., an antibody or antigen binding fragment thereof provided herein), or a composition provided herein that will be effective in the prevention and/or treatment of a disease or condition can be determined by standard clinical techniques. In addition, in vitro assays may optionally be employed to help identify optimal dosage ranges. The precise dose to be employed in the formulation will also depend on the route of administration, and the seriousness of a disease or condition, and should be decided according to the judgment of the practitioner and each patient's circumstances.

Effective doses may be extrapolated from dose-response curves derived from in vitro or animal model test systems.

In certain embodiments, the route of administration for a dose of an antibody or antigen binding fragment thereof provided herein to a patient is intranasal, intramuscular, intravenous, subcutaneous, or a combination thereof, but other routes described herein are also acceptable. Each dose may or may not be administered by an identical route of administration. In some embodiments, an antibody or antigen binding fragment thereof provided herein may be administered via multiple routes of administration simultaneously or subsequently to other doses of the same or a different antibody or antigen binding fragment thereof provided herein.

In certain embodiments, the multispecific antibody or antigen binding fragment thereof or fusion construct provided herein are administered prophylactically or therapeutically to a subject. The multispecific antibody or antigen binding fragment thereof provided herein can be prophylactically or therapeutically administered to a subject so as to prevent, lessen or ameliorate a disease or symptom thereof.

### Diagnostic Assays and Methods

Labeled antibodies, derivatives, and analogs thereof, which immunospecifically bind to a HER2 antigen can be used for diagnostic purposes to detect, diagnose, or monitor a HER2-mediated disease. Thus, provided herein are methods for the detection of a HER2-mediated disease comprising: (a) assaying the expression of a HER2 antigen in cells or a tissue sample of a subject using one or more antibodies, provided herein that immunospecifically bind to the HER2 antigen; and (b) comparing the level of the HER2 antigen with a control level, e.g., levels in normal tissue samples (e.g., from a patient not having a HER2-mediated disease, or from the same patient before disease onset), whereby an increase in the assayed level of HER2 antigen compared to the control level of the HER2 antigen is indicative of a HER2-mediated disease.

Also provided herein is a diagnostic assay for diagnosing a HER2-mediated disease comprising: (a) assaying for the level of a HER2 antigen in cells or a tissue sample of an individual using one or more antibodies, provided herein that immunospecifically bind to a HER2 antigen; and (b) comparing the level of the HER2 antigen with a control level, e.g., levels in normal tissue samples, whereby an increase in the assayed HER2 antigen level compared to the control level of the HER2 antigen is indicative of a HER2-mediated disease. In certain embodiments, provided herein is a method of treating a HER2-mediated disease in a subject, comprising: (a) assaying for the level of a HER2 antigen in cells or a tissue sample of the subject using one or more antibodies, provided herein that immunospecifically bind to a HER2 antigen; and (b) comparing the level of the HER2 antigen with a control level, e.g., levels in normal tissue samples, whereby an increase in the assayed HER2 antigen level compared to the control level of the HER2 antigen is indicative of a HER2-mediated disease. In some embodiments, the method further comprises (c) administering an effective amount of an antibody, provided herein to the subject identified as having the HER2-mediated disease. A more definitive diagnosis of a HER2-mediated disease may allow health professionals to employ preventative measures or aggressive treatment earlier thereby preventing the development or further progression of the HER2-mediated disease.

Antibodies provided herein can be used to assay HER2 antigen levels in a biological sample using classical immunohistological methods as described herein or as known to those of skill in the art (e.g., see Jalkanen et al., 1985, J. Cell. Biol. 101:976-985; and Jalkanen et al., 1987, J. Cell. Biol. 105:3087-3096). Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase; radioisotopes, such as iodine (125I, 121I), carbon (14C), sulfur (35S), tritium (3H), indium (121In), and technetium (99Tc); luminescent labels, such as luminol; and fluorescent labels, such as fluorescein and rhodamine, and biotin.

One aspect provided herein is the detection and diagnosis of a HER2-mediated disease in a human. In one embodiment, diagnosis comprises: a) administering (for example, parenterally, subcutaneously, or intraperitoneally) to a subject an effective amount of a labeled antibody, that immunospecifically binds to a HER2 antigen; b) waiting for a time interval following the administering for permitting the labeled antibody to concentrate at sites in the subject where the HER2 antigen is expressed (and for unbound labeled molecule to be cleared to background level); c) determining background level; and d) detecting the labeled antibody, in the subject, such that detection of labeled antibody above the background level indicates that the subject has a HER2-mediated disease. Background level can be determined by various methods including, comparing the amount of labeled molecule detected to a standard value previously determined for a particular system.

It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of 99Tc. The labeled antibody will then accumulate at the location of cells which contain the specific protein. In vivo tumor imaging is described in S. W. Burchiel et al., “Immunopharmacokinetics of Radiolabeled Antibodies and Their Fragments.” (Chapter 13 in Tumor Imaging: The Radiochemical Detection of Cancer, S. W. Burchiel and B. A. Rhodes, eds., Masson Publishing Inc. (1982).

Depending on several variables, including the type of label used and the mode of administration, the time interval following the administration for permitting the labeled antibody, to concentrate at sites in the subject and for unbound labeled antibody, to be cleared to background level is 6 to 48 hours or 6 to 24 hours or 6 to 12 hours. In another embodiment the time interval following administration is 5 to 20 days or 5 to 10 days.

In one embodiment, monitoring of a HER2-mediated disease is carried out by repeating the method for diagnosing a HER2-mediated disease, for example, one month after initial diagnosis, six months after initial diagnosis, one year after initial diagnosis, etc.

Presence of the labeled molecule can be detected in the subject using methods known in the art for in vivo scanning. These methods depend upon the type of label used. Skilled artisans will be able to determine the appropriate method for detecting a particular label. Methods and devices that may be used in the diagnostic methods provided herein include, but are not limited to, computed tomography (CT), whole body scan such as position emission tomography (PET), magnetic resonance imaging (MRI), and sonography.

In a specific embodiment, the molecule is labeled with a radioisotope and is detected in the patient using a radiation responsive surgical instrument (Thurston et al., U.S. Pat. No. 5,441,050). In another embodiment, the molecule is labeled with a fluorescent compound and is detected in the patient using a fluorescence responsive scanning instrument. In another embodiment, the molecule is labeled with a positron emitting metal and is detected in the patient using positron emission-tomography. In yet another embodiment, the molecule is labeled with a paramagnetic label and is detected in a patient using magnetic resonance imaging (MRI).

### Kits

Also provided herein are kits comprising a multispecific antibody (e.g., an anti-HER2 antibody) provided herein, or a composition (e.g., a pharmaceutical composition) packaged into suitable packaging material. A kit optionally includes a label or packaging insert including a description of the components or instructions for use in vitro, in vivo, or ex vivo, of the components therein.

The term “packaging material” refers to a physical structure housing the components of the kit. The packaging material can maintain the components sterilely, and can be made of material commonly used for such purposes (e.g., paper, corrugated fiber, glass, plastic, foil, ampoules, vials, tubes, etc.).

Kits provided herein can include labels or inserts. Labels or inserts include “printed matter,” e.g., paper or cardboard, separate or affixed to a component, a kit or packing material (e.g., a box), or attached to, for example, an ampoule, tube, or vial containing a kit component. Labels or inserts can additionally include a computer readable medium, such as a disk (e.g., hard disk, card, memory disk), optical disk such as CD- or DVD-ROM/RAM, DVD, MP3, magnetic tape, or an electrical storage media such as RAM and ROM or hybrids of these such as magnetic/optical storage media, FLASH media, or memory type cards. Labels or inserts can include information identifying manufacturer information, lot numbers, manufacturer location, and date.

Kits provided herein can additionally include other components. Each component of the kit can be enclosed within an individual container, and all of the various containers can be within a single package. Kits can also be designed for cold storage. A kit can further be designed to contain antibodies provided herein, or cells that contain nucleic acids encoding the antibodies provided herein. The cells in the kit can be maintained under appropriate storage conditions until ready to use.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described herein.

As used herein, numerical values are often presented in a range format throughout this document. The use of a range format is merely for convenience and brevity and should not be construed as an inflexible limitation on the scope of the invention unless the context clearly indicates otherwise. Accordingly, the use of a range expressly includes all possible subranges, all individual numerical values within that range, and all numerical values or numerical ranges including integers within such ranges and fractions of the values or the integers within ranges unless the context clearly indicates otherwise. This construction applies regardless of the breadth of the range and in all contexts throughout this patent document. Thus, for example, reference to a range of 90-100% includes 91-99%, 92-98%, 93-95%, 91-98%, 91-97%, 91-96%, 91-95%, 91-94%, 91-93%, and so forth. Reference to a range of 90-100% also includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth.

In addition, reference to a range of 1-3, 3-5, 5-10, 10-20, 20-30, 30-40, 40-50, 50-60, 60-70, 70-80, 80-90, 90-100, 100-110, 110-120, 120-130, 130-140, 140-150, 150-160, 160-170, 170-180, 180-190, 190-200, 200-225, 225-250 includes 1, 2, 3, 4, 5, 6, 7, 8, 9, 10, 11, 12, 13, 14, 15, 16, 17, 18, 19, 20, etc. In a further example, reference to a range of 25-250, 250-500, 500-1,000, 1,000-2,500, 2,500-5,000, 5,000-25,000, 25,000-50,000 includes any numerical value or range within or encompassing such values, e.g., 25, 26, 27, 28, 29 . . . 250, 251, 252, 253, 254 . . . 500, 501, 502, 503, 504 . . . , etc.

As also used herein a series of ranges are disclosed throughout this document. The use of a series of ranges include combinations of the upper and lower ranges to provide another range. This construction applies regardless of the breadth of the range and in all contexts throughout this patent document. Thus, for example, reference to a series of ranges such as 5-10, 10-20, 20-30, 30-40, 40-50, 50-75, 75-100, 100-150, includes ranges such as 5-20, 5-30, 5-40, 5-50, 5-75, 5-100, 5-150, and 10-30, 10-40, 10-50, 10-75, 10-100, 10-150, and 20-40, 20-50, 20-75, 20-100, 20-150, and so forth.

For the sake of conciseness, certain abbreviations are used herein. One example is the single letter abbreviation to represent amino acid residues. The amino acids and their corresponding three letter and single letter abbreviations are as follows:

| | | |
|---|---|---|
| alanine | Ala | (A) |
| arginine | Arg | (R) |
| asparagine | Asn | (N) |
| aspartic acid | Asp | (D) |
| cysteine | Cys | (C) |
| glutamic acid | Glu | (E) |
| glutamine | Gln | (Q) |
| glycine | Gly | (G) |
| histidine | His | (H) |
| isoleucine | Ile | (I) |
| leucine | Leu | (L) |
| lysine | Lys | (K) |
| methionine | Met | (M) |
| phenylalanine | Phe | (F) |
| proline | Pro | (P) |
| serine | Ser | (S) |
| threonine | Thr | (T) |
| tryptophan | Trp | (W) |
| tyrosine | Tyr | (Y) |
| valine | Val | (V) |

The invention is generally disclosed herein using affirmative language to describe the numerous embodiments. The invention also specifically includes embodiments in which particular subject matter is excluded, in full or in part, such as substances or materials, method steps and conditions, protocols, procedures, assays or analysis. Thus, even though the invention is generally not expressed herein in terms of what the invention does not include, aspects that are not expressly included in the invention are nevertheless disclosed herein.

A number of embodiments of the invention have been described. Nevertheless, it will be understood that various modifications may be made without departing from the spirit and scope of the invention. Accordingly, the following examples are intended to illustrate but not limit the scope of invention described in the claims.

### EMBODIMENTS

The disclosure provided herein also provides the following non-limiting embodiments.
1. A multispecific antibody or antigen binding fragment thereof comprising at least one of a first antigen-binding region and a second antigen-binding region each capable of binding specifically to human epidermal growth factor receptor 2 (HER2), and a third antigen-binding region capable of binding specifically to CD98, wherein:
   (1) the first antigen-binding region comprises:
a first heavy chain variable region (VH1) comprising a heavy chain complementarity determining region 1 (HCDR1), a HCDR2, and a HCDR3 comprising the amino acid sequences of SEQ ID NOs: 5, 6 and 7, respectively; and a first light chain variable region (VL1) comprising a light chain complementarity determining region 1 (LCDR1), a LCDR2, and a LCDR3 comprising the amino acid sequences of SEQ ID NOs: 8, 9 and 10, respectively;
   (2) the second antigen-binding region comprises:
second heavy chain variable region (VH2) comprising a heavy chain complementarity determining region 1 (HCDR1), a HCDR2, and a HCDR3 comprising the amino acid sequences of SEQ ID NOs: 13, 14 and 15, respectively; and a second light chain variable region (VL2) comprising a light chain complementarity determining region 1 (LCDR1), a LCDR2, and a LCDR3 comprising the amino acid sequences of SEQ ID NOs: 16, 17 and 18, respectively; and
   (3) the third antigen-binding region comprises a first single chain variable fragment (scFv1) having: a third heavy chain variable region (VH3) comprising a heavy chain complementarity determining region 1 (HCDR1), a HCDR2, and a HCDR3, and a third light chain variable region (VL3) comprising a light chain complementarity determining region 1 (LCDR1), a LCDR2, and a LCDR3, wherein the HCDR1, HCDR2, HCDR3, LCDR1, LCDR2 and LCDR3 have any of the amino acid sequences of Table 2.
2. The multispecific antibody or antigen binding fragment thereof of embodiment 1, wherein:
   (1) the VH1 an amino acid sequence identical to the VH of the HC sequence identified as SEQ ID NO: 1; and the VL1 comprises an amino acid sequence identical to the VL of the LC sequence identified as SEQ ID NO: 2;
   (2) the VH2 an amino acid sequence identical to the VH of the HC sequence identified as SEQ ID NO: 11; and the VL2 comprises an amino acid sequence identical to the VL of the LC sequence identified as SEQ ID NO: 12; and
   (3) the VH3 and the VL3 comprise amino acid sequences are identical to the respective VH and VL of a scFv identified as:
      (xxii) SEQ ID NO: 19;
      (xxiii) SEQ ID NO: 26;
      (xxiv) SEQ ID NO: 33;
      (XXV) SEQ ID NO: 34;
      (xxvi) SEQ ID NO: 35;
      (xxvii) SEQ ID NO: 36;
      (xxviii) SEQ ID NO: 43;
      (xxix) SEQ ID NO: 49;
      (xxx) SEQ ID NO: 55;
      (xxxi) SEQ ID NO: 59;
      (xxxii) SEQ ID NO: 66;
      (xxxiii) SEQ ID NO: 71;
      (xxxiv) SEQ ID NO: 78;
      (XXXV) SEQ ID NO: 85;
      (xxxvi) SEQ ID NO: 87;
      (xxxvii) SEQ ID NO: 88;
      (xxxviii) SEQ ID NO: 89;
      (xxxix) SEQ ID NO: 90;
      (xl) SEQ ID NO: 92;
      (xli) SEQ ID NO: 93;
      (xlii) SEQ ID NO: 94.
3. The multispecific antibody or antigen binding fragment thereof of embodiment 1 or 2, comprising:
   (a) a first heavy chain (HC1) comprising the VH1, a first heavy chain constant region comprising a first Fc region (Fc1), and the scFv1, and
   (b) a first light chain (LC1) comprising the VL1 and a light chain constant region.
4. The multispecific antibody or antigen binding fragment thereof of embodiment 3, further comprising:
   (a) a second heavy chain (HC2) comprising the VH1 and a first heavy chain constant region comprising a second Fc region (Fc2), and
   (b) a second light chain (LC2) comprising the VL1 and a light chain constant region.
5. The multispecific antibody or antigen binding fragment thereof of claim 3, further comprising a second Fc region (Fc2).
6. The multispecific antibody or antigen binding fragment thereof of embodiment 1 or 2, comprising:
   (a) a first heavy chain (HC1) comprising the VH1, and a first heavy chain constant region comprising a first Fc region (Fc1),
   (b) a first light chain (LC1) comprising the VL1 and a light chain constant region; and
   (c) a second heavy chain (HC2) comprising the scFv1, and a second heavy chain constant region comprising a second Fc region (Fc2).
7. The multispecific antibody or antigen binding fragment thereof of embodiment 1 or 2, comprising:
   (a) a first heavy chain (HC1) comprising the VH2, a first heavy chain constant region comprising a first Fc region (Fc1), and the scFv1, and
   (b) a first light chain (LC1) comprising the VL2 and a light chain constant region.
8. The multispecific antibody or antigen binding fragment thereof of embodiment 7, further comprising:
   (a) a second heavy chain (HC2) comprising the VH2 and a first heavy chain constant region comprising a second Fc region (Fc2), and
   (b) a second light chain (LC2) comprising the VL2 and a light chain constant region.
9. The multispecific antibody or antigen binding fragment thereof of embodiment 7, further comprising a second Fc region (Fc2).
10. The multispecific antibody or antigen binding fragment thereof of embodiment 1 or 2, comprising:
   (a) a first heavy chain (HC1) comprising the VH2, and a first heavy chain constant region comprising a first Fc region (Fc1),
   (b) a first light chain (LC1) comprising the VL2 and a light chain constant region; and
   (c) a second heavy chain (HC2) comprising the scFv1, and a second heavy chain constant region comprising a second Fc region (Fc2).
11. The multispecific antibody or antigen binding fragment thereof of embodiment 1 or 2, comprising the first antigen binding region, the second antigen binding region and the third antigen binding region.
12. The multispecific antibody or antigen binding fragment thereof of embodiment 11, comprising:
   (a) a first heavy chain (HC1) comprising the VH1, a first heavy chain constant region comprising a first Fc region (Fc1), and the scFv1,
   (b) a first light chain (LC1) comprising the VL1 and a light chain constant region; and
   (c) a second heavy chain (HC2) comprising a second single chain variable fragment (scFv2) and a first heavy chain constant region comprising a second Fc region (Fc2), wherein the scFv2 comprises the VH2 and VL2.
13. The multispecific antibody or antigen binding fragment thereof of embodiment 11, comprising:
   (a) a first heavy chain (HC1) comprising the VH2, a first heavy chain constant region comprising a first Fc region (Fc1), and the scFv1,
   (b) a first light chain (LC1) comprising the VL2 and a light chain constant region; and
   (c) a second heavy chain (HC2) comprising a second single chain variable fragment (scFv2) and a first heavy chain constant region comprising a second Fc region (Fc2), wherein the scFv2 comprises the VH1 and VL1.
14. The multispecific antibody or antigen binding fragment thereof of any of the foregoing embodiments, wherein the scFv1 and/or scFv2 comprises at least one of (a) a first disulfide bond between a structurally conserved surface exposed VH cysteine (Cys) and a first L Cys; and b) a second disulfide bond between a structurally conserved surface exposed VL Cys and a second L Cys.
15. The multispecific antibody or antigen binding fragment thereof of embodiment 14, wherein the scFv1 and scFv2 each independently comprise the first disulfide bond and the second disulfide bond.
16. The multispecific antibody or antigen binding fragment thereof of embodiment 13, wherein the scFv2 comprises an amino acid sequence having an amino acid sequence of SEQ ID NO: 3 or 4.
17. The multispecific antibody or antigen binding fragment thereof of any one of the foregoing embodiments, wherein the scFv1 comprises an amino acid sequence selected from the group consisting of: SEQ ID NO: SEQ ID NO: 19, SEQ ID NO: 26, SEQ ID NO: 33, SEQ ID NO: 34, SEQ ID NO: 35, SEQ ID NO: 36, SEQ ID NO: 43, SEQ ID NO: 49, SEQ ID NO: 55, SEQ ID NO: 59, SEQ ID NO: 66, SEQ ID NO: 71, SEQ ID NO: 78, SEQ ID NO: 85, SEQ ID NO: 87, SEQ ID NO: 88, SEQ ID NO: 89, SEQ ID NO: 90, SEQ ID NO: 92, SEQ ID NO: 93, SEQ ID NO: 94.
18. The multispecific antibody or antigen binding fragment thereof of any one of embodiments 4-6, 8-10 and 12-17, wherein each of the Fc1 and Fc2 comprises one or more heterodimeric mutations, or one or more knob and hole mutations.
19. The multispecific antibody or antigen binding fragment thereof of embodiment 18, wherein the heterodimeric mutations comprise amino acid modifications at positions T350, L351, F405, and Y407 in one of Fc1 and Fc2, and amino acid modifications at positions T350, T366, K392 and T394 in the other one of Fc1 and Fc2, wherein the amino acid modification at position T350 is T350V, T350I, T350L or T350M; the amino acid modification at position L351 is L351Y; the amino acid modification at position F405 is F405A, F405V, F405T or F405S; the amino acid modification at position Y407 is Y407V, Y407A or Y407I; the amino acid modification at position T366 is T366L, T366I, T366V or T366M, the amino acid modification at position K392 is K392F, K392L or K392M, and the amino acid modification at position T394 is T394W, and wherein the numbering of amino acid residues is according to the EU index as set forth in Kabat.
20. The multispecific antibody or antigen binding fragment thereof of embodiment 19, wherein one of Fc1 and Fc2 comprises mutations T350V, L351Y, F405A and Y407V, and the other of Fc1 and Fc2 comprises mutations T350V, T366L, K392L and T394W.
21. The multispecific antibody or antigen binding fragment thereof of embodiment 18, wherein each of the Fc1 and Fc2 comprises one or more knob and hole mutations.
22. The multispecific antibody or antigen binding fragment thereof of any one of the foregoing embodiments, comprising a Fc domain having amino acid modifications that enhance binding of the multispecific antibody or antigen binding fragment thereof to a neonatal Fc receptor (RcRn), preferably the amino acid modifications enhance the binding at an acidic pH, more preferably the Fc domain has the M252Y/S254T/T256E (YTE) mutations, wherein the numbering of amino acid residues is according to the EU index as set forth in Kabat.
23. The multispecific antibody or antigen binding fragment thereof of any one of the foregoing embodiments, comprising a Fc domain having amino acid modifications that reduce or eliminate the effector function, preferably the Fc domain has one or more amino acid modifications at positions L234, L235, D265, D270, N297, E318, K320, K322, P331, and P329, such as one, two, three or four amino acid modifications of L234A, L235A, D265S and P331S, wherein the numbering of amino acid residues is according to the EU index as set forth in Kabat.
24. The multispecific antibody or antigen binding fragment thereof of any one of the foregoing embodiments, comprising a Fc domain having one or more amino acid modifications of M252Y, S254T, T256E, L234A, L235A and D265S, wherein the numbering of amino acid residues is according to the EU index as set forth in Kabat.
25. The multispecific antibody or antigen binding fragment thereof of any one of the foregoing embodiments, comprising a Fc domain not having amino acid modifications that reduce or eliminate the effector function.
26. A multispecific antibody comprising a first heavy chain, a light chain and a second heavy chain each having the amino acid sequences at least 90% identical to
   (1) SEQ ID NO: 100, SEQ ID NO: 12, and SEQ ID NO: 101, respectively; or
   (2) SEQ ID NO: 102, SEQ ID NO: 12, and SEQ ID NO: 103, respectively;
wherein the first antigen-binding region is capable of binding specifically to a first epitope of HER2, the second antigen-binding region is capable of binding specifically to a second epitope of HER2, and the third antigen-binding region is capable of binding specifically to CD98.
27. The multispecific antibody of embodiment 26, wherein the first heavy chain, the light chain and the second heavy chain each comprises the amino acid sequences of:
   (1) SEQ ID NO: 100, SEQ ID NO: 12, and SEQ ID NO: 101, respectively; or
   (2) SEQ ID NO: 102, SEQ ID NO: 12, and SEQ ID NO: 103, respectively.
28. An isolated nucleic acid encoding the multispecific antibody or antigen binding fragment thereof of any one of embodiments 1-27.
29. A vector comprising the isolated nucleic acid of embodiment 28.
30. A host cell comprising the isolated nucleic acid of embodiment 28 or the vector of embodiment 29.
31. A method of producing a multispecific antibody or antigen binding fragment thereof, the method comprising culturing the host cell of embodiment 30 under conditions to produce the multispecific antibody or antigen binding fragment thereof and recovering the multispecific antibody or antigen binding fragment thereof.
32. A pharmaceutical composition comprising the multispecific antibody or antigen binding fragment thereof of any one of embodiments 1-27 and a pharmaceutically acceptable carrier.
33. A method of treating or detecting a disorder, preferably a cancer, in a subject in need thereof, comprising administering to the subject the multispecific antibody or antigen-binding fragment of any one of embodiments 1-27, or the pharmaceutical composition of embodiment 32.
34. The method of embodiment 33, wherein the disease or disorder is a HER2 associated disease or disorder.
35. The method of embodiment 34, wherein the disease or disorder is brain metastasis.
36. A pharmaceutical composition comprising the isolated nucleic acid of claim 28, the vector of claim 29 or the host cell of claim 30 and a pharmaceutically acceptable carrier.
37. A method of treating a disorder, preferably a cancer, in a subject in need thereof, comprising administering to the subject the pharmaceutical composition of embodiment 36.
38. The method of embodiment 37, wherein the disease or disorder is a HER2 associated disease or disorder.
39. The method of embodiment 37, wherein the disease or disorder is brain metastasis.

### EXAMPLES

The following is a description of various methods and materials used in the studies, and are put forth so as to provide those of ordinary skill in the art with a complete disclosure and description of how to make and use the present disclosure, and are not intended to limit the scope of what the inventors regard as their disclosure nor are they intended to represent that the experiments below were performed and are all of the experiments that may be performed. It is to be understood that exemplary descriptions written in the present tense were not necessarily performed, but rather that the descriptions can be performed to generate the data and the like associated with the teachings of the present disclosure. Efforts have been made to ensure accuracy with respect to numbers used (e.g., amounts, percentages, etc.), but some experimental errors and deviations should be accounted for.

### Example 1. Materials and Methods

### CD98 Antibody Generation and Screening

Transgenic rodents were immunized with CD98 proteins with the repetitive immunization at multiple sites (RIMMS) protocol. Serum titers were assessed by ELISA to select the rodents for fusion. Lymph nodes were harvested from sera-positive rodents and fused with myeloma cells to generate hybridomas using standard methods. Supernatant hybridomas were screened by MSD for binding to proteins, CD98 expressing cells, and human brain endothelial cells. Positive clones were further assessed for internalization using brain endothelial cells, cross-reactivity to human and cyno proteins, or CD98/LAT1 transport activity. CD98 clones that bound both human and cyno CD98, internalized without interfering with CD98/LAT1 activity, were selected for variable sequence recovery and converted to scFv to generate TEM mAbs for further characterizations.

### Bispecific TEM Antibody Generation and Characterization

Anti-HER2 and anti-CD98 were used to generate bispecific antibodies using the knobs-into-holes heterodimerization technology (Ridgway et al., 1996, Protein Eng. 9, 617-621). The anti-HER2 binders are made as bivalent Fab, monovalent Fab, or monovalent scFv onto the N-terminus of the Fc, whereas the anti-CD98 binders are made as monovalent scFv for attachment to the C-terminus of one heavy chain Fc through a (G4S) 4 linker. In addition to the knob and hole mutations in the Fc, all antibodies except BBBB1819 contain mutations that enhance FcRn binding at acidic pH [M252Y/S254T/T256E (YTE)] for half-life extension (HLE). The BBBB1613 and BBBB1636 antibodies also contain mutations in the Fc that abrogated effector function [L234A/L235A/D265S (AAS)]. The constructed TEM mAbs were expressed in ExpiCHO-S™ cells by transient transfection with purified plasmid DNA following the manufacturer's recommendations. The harvested cell culture supernatants were purified by Protein A and cation exchange chromatography. Homogeneity and purity of the final antibodies were confirmed by SDS-PAGE, analytical size exclusion chromatography and mass spectrometry.

TABLE 4
TEM antibodies

| | | | | | | | | anti-HER2 | | anti-HER2 | |
|---|---|---|---|---|---|---|---|---|---|---|---|
| | | | TEM | | | | | HC1 | | HC2 | |
| B# | Fc | HLE | Format | ID | — | His-cap? | location | binder | Format | binder | Format |
| BBBB1819 | WT | none | spFv | CD98hc11sp-H | human | Yes | C-term | Pert | Fab | Trast | spFv |
| | | | | | | | HC1 | | | | |
| BBBB1809 | WT | YTE | spFv | CD98hc11sp-H | human | Yes | C-term | Pert | Fab | Trast | spFv |
| | | | | | | | HC1 | | | | |
| BBBB1615 | WT | YTE | scFv | CD98hc2 | cyno | no | C-term | Pert | Fab | Trast | scFv |
| | | | | | surrogate | | HC1 | | | | |
| BBBB1616 | WT | YTE | scFv | CD98hc11 | human | no | C-term | Pert | Fab | Trast | scFv |
| | | | | | | | HC1 | | | | |
| BBBB1617 | WT | YTE | scFv | CD98hc12 | weaker | no | C-term | Pert | Fab | Trast | scFv |
| | | | | | human | | HC1 | | | | |
| BBBB1826 | WT | YTE | scFv | CD98hc11-H | human | Yes | C-term | Pert | Fab | Trast | scFv |
| | | | | | | | HC1 | | | | |
| BBBB1805 | WT | YTE | spFv | CD98hc11sp | human | no | C-term | Pert | Fab | Trast | spFv |
| | | | | | | | HC1 | | | | |
| BBBB1640 | WT | YTE | scFv | CD98hc2 | cyno | no | C-term | Trast | Fab | Trast | Fab |
| | | | | | surrogate | | HC1 | | | | |
| BBBB1639 | WT | YTE | scFv | CD98hc12 | weaker | no | C-term | Trast | Fab | Trast | Fab |
| | | | | | human | | HC1 | | | | |
| BBBB1613 | AAS | YTE | scFv | CD98hc2 | cyno | no | C-term | Pert | Fab | Trast | scFv |
| | | | | | surrogate | | HC1 | | | | |
| BBBB1636 | AAS | YTE | scFv | CD98hc2 | cyno | no | C-term | Trast | Fab | Trast | Fab |
| | | | | | surrogate | | HC1 | | | | |
| BBBB1614 | WT | YTE | scFv | CD98hc2 | cyno | no | N-term | Trast | Fab | empty | |
| | | | | | surrogate | | HC2 | | | | |
| ERBB2077 | WT | none | N/A | N/A | N/A | no | N/A | Pert | Fab | Trast | spFv |

Culture and maintenance of HER2-expressing cancer cells. BT-474 (HTB-20), BT-474 Clone 5 (CRL-3247), HCC-1954 (CRL-2338) and MDA-MB-361 (HTB-27) and U87-MG (HTB-14) were obtained from ATCC. JIMT-1 cells were obtained from Creative Bioarray. U87-MG cells were cultured in EMEM supplemented with 10% low IgG FBS. All other cells were cultured in RPMI supplemented with 10% low IgG FBS and 1% non-essential amino acids (BT474 and BT-474 Clone 5 only). For imaging studies cells were transduced with Incucyte® NucLight Red (Essen Biosciences #4476) or rFLuc-T2A-GFP (System Biosciences #LL410VA-1) Lentivirus. Stable cell lines were selected for and maintained in medium containing 0.5 μg/mL (BT-474, BT-474 Clone5, MDA-MB-361) or 1 μg/mL Puromycin (HCC-1954, JIMT-1).

Antibody binding to cells. MDA-MB-361, BT474, HCC-1954, JIMT-1 and U87-MG cells were detached with accutase, washed twice with cold DPBS, and concentration adjusted to 2×10⁶ cells/mL in cold binding buffer (BD binding buffer with 2 mM EDTA), then added 50 μL/well in 96-well plates. 50 μL/well of antibody solution in cold binding buffer was added, plates were covered, and incubated at 4° C. for 1 h. Wells were subsequently washed with 100 μL/well cold DPBS and spun at 300×g for 5 min to remove supernatants. Next, pellets were resuspended in 100 μL/well LIVE/DEAD Fixable Dead Cell Dye (Invitrogen, 1/1,000 dilution) in cold DPBS and incubated 30 min on ice. Cells were washed two more time in binding buffer as above, resuspended in a final volume of 50 μL/well, and read at the iQue flow cytometer (Sartorius).

Human iPSC-derived microglia culture. Human microglia were derived from epithelial-derived iPSCs (IPSC0028, male, Sigma) and were handled as described previously {Haenseler, 2017 #607}. Briefly, iPSCs were plated into Aggrewell 800 plates and allowed to form embryonic bodies (EBs) to recapitulate the microglial development in embryos. Macrophage precursor cells were generated by culturing EBs in mTESR1 with bone morphogenetic protein 4 (BMP4, 50 ng/ml;), vascular endothelial growth factor (VEGF, 50 ng/mL) and stem cell factor (SCF, 25 ng/mL) for 3 days. EBs were then harvested and transferred to a six-well plate and cultured for 8 weeks in EX-VIVO15 (Lonza) supplemented with Glutamax, penicillin/streptomycin, β-mercaptoethanol, IL-3 (25 ng/ml) and M-CSF (100 ng/ml) to promote myeloid differentiation. Excreted macrophage precursors were collected from the supernatant and plated at 20000 cells per well in 96-well plate, where they were allowed to mature for 14 days in advanced DMEM/F12 supplemented with Glutamax, penicillin/streptomycin, β-mercaptoethanol, IL-34 (100 ng/ml) and GM-SCF (10 ng/ml). Microglia were characterized by positive immunostaining with antibodies to the following proteins: Iba1 (019-19741; 1:500; Wako), P2RY12 (HPA014518; 1:100; Sigma), CX3CR1 (2091; 1:200; ProSci Inc.), CD11b (MAB1699; 1:500, RnD Systems) and CB68 (M078, 1:500, Dako).

pH-rodo cell phagocytosis. Target cell lines were labeled with pHrodo® Red according to manufacturer's instructions (Essen Biosciences, 4649). pHrodo® labeled cells were opsonized with TEM mAbs or control mAbs at 37° C. for 30 min, before coculture with either iPSC-derived microglia at various E:T ratios. Microglia cocultures were monitored in Incucyte® SX5 imaging every 90 min, and cell phagocytosis was measured as Total pHrodoRed Area (μm²/Image) and total pHrodoRed Integrated Intensity (RCU×μm²/Image).

Co-culture killing assay. NucLight Red labeled target cell lines were detached, washed and opsonized with TEM mAbs or control mAbs at 37° C. for 30 min, before coculture with iPSC-derived microglia. Co-cultures were observed by imaging in an Incucyte® SX5. Target cell loss was determined by quantification of NucLight Red area per image.

### Non-Human Primate Cynomolgus Monkey PK Studies

Study design. TEM and control IgG1 mAbs were administered into cynomolgus monkeys at 10 mg/kg via slow bolus IV injection. Blood for PK was collected at 1, 6, 24, 72 and 168 h post dosing and processed to serum via the testing facility lab protocol. For brain tissue collection, cynomolgus monkeys were placed under deep anesthesia, and terminal blood draw was performed. Following terminal blood collection, animals were euthanized at 72 and 168 h (n=2 at each time point), and upper body perfusion was performed according to the test facility standard operating procedures (SOP) by perfusing cold saline solution for a minimum of 5 min at 250 mL/min. Approximately 200 mg of tissue was isolated from predefined brain locations (frontal lobe, hippocampus and temporal lobe), snap frozen in liquid nitrogen and stored at −70° C. until capillary depletion processing and tissue homogenization.

Brain tissue preparation. Each right/left hemisphere was weighed and processed to capillary-depleted brain tissue as previously described, with some modifications.²⁵ Briefly, brain tissue samples were slowly thawed on wet ice, added to a calculated volume (2.5 μL buffer/1 mg tissue) of a modified DPBS buffer containing protease inhibitor (Pierce; A32955) and transferred to Lysing Matrix D tubes (MP Biomedicals™; 6913-100). Total cell suspensions were produced by homogenizing the tissue at 2.8 m/s for 15 seconds with a Bead Ruptor 24 Elite (Omni International). The total cell suspension was transferred into a new tube and mixed with an equal volume of dextran buffer (Sigma; 31397) to a 13% final dextran concentration. Dextran-containing cell suspension was centrifuged at 2,000 g for 20 min at 4° C. The upper layer (capillary-depleted fraction) was carefully separated from the remaining sample and transferred to a new tube containing 10× radioimmune precipitation assay (RIPA) lysis buffer (Millipore™; 20-188). Capillary-depleted samples plus lysis buffer were vortexed well and centrifuged at 14,000 rpm for 30 min at 4° C., and supernatant was collected for analysis. Processed brain tissue lysates were tested for protein concentrations with a BCA protein assay kit (Pierce™; 23227), and final sample lysates were normalized to 7 mg/mL total protein concentration before immunoassay determination.

PK assays. The concentrations of TEM and control IgG1 mAbs in NHP brain tissue and plasma were determined with MSD immunoassays on small-spot streptavidin plates. Fresh standard curves were prepared by serial dilution for each mAb in assay diluent containing naïve mouse matrix (50% brain tissue lysates or 10% pooled plasma). Frozen quality controls prepared in 100% naïve mouse matrix were diluted and tested with each assay. Briefly, plates were blocked with 1% bovine serum albumin in PBS for 30 min and washed with 0.05% Tween-20 in PBS. Master mix containing capture and detection reagents (biotinylated and ruthenium-labeled anti-human Fc mAb) was combined in the assay plate with reference standards, quality controls, and samples at a 1:1 volume ratio and incubated for 1 h with shaking. The raw data signal was read on a Meso Sector S 600 imager and analyzed by the Watson LIMS software (Thermo Scientific). Data regression was performed with a five-parameter logistic fit with 1/Y² weighting. The quantifiable curve range for the brain tissue lysate assay was 1-512 ng/mL with a minimum required sample dilution of 1:2 for processed tissue. To calculate the total tissue drug concentration in brain tissue, the mAb concentration in brain lysates was multiplied by the total volume used to process to the final 7 mg/mL normalized sample. The total tissue drug concentration (ng) was then divided by the brain tissue wet weight to determine the drug: tissue (ng: mg) ratio. The quantifiable curve range for the plasma assay was 2-512 ng/mL with a minimum required sample dilution of 1:10. The assay had sensitivity limit of 2 ng/mL in brain tissue lysates and 10 ng/mL in plasma.

### Example 2. Antibody Binding in HER2-Positive Cancer Cells

To determine the ability of TEM mAb to mediate clearance of cells, TEM constructs were generated using the anti-human epidermal growth factor receptor 2 (HER2) binding antibody Trastuzumab (bivalent BBBB1639 and BBBB1640; and bivalent BBBB1636 with no Fc mediated effector function) as well as the biparatopic molecule containing both Trastuzumab and Pertuzumab (biparatopic BBBB1819, BBBB1809, BBBB1615, BBBB1616, BBBB1617, BBBB1826 and BBBB1805; and biparatopic BBBB1613 with no Fc mediated effector function) (FIG. 2B). Both Trastuzumab and Pertuzumab have been extensively characterized both in vitro and in vivo and are FDA approved drugs.

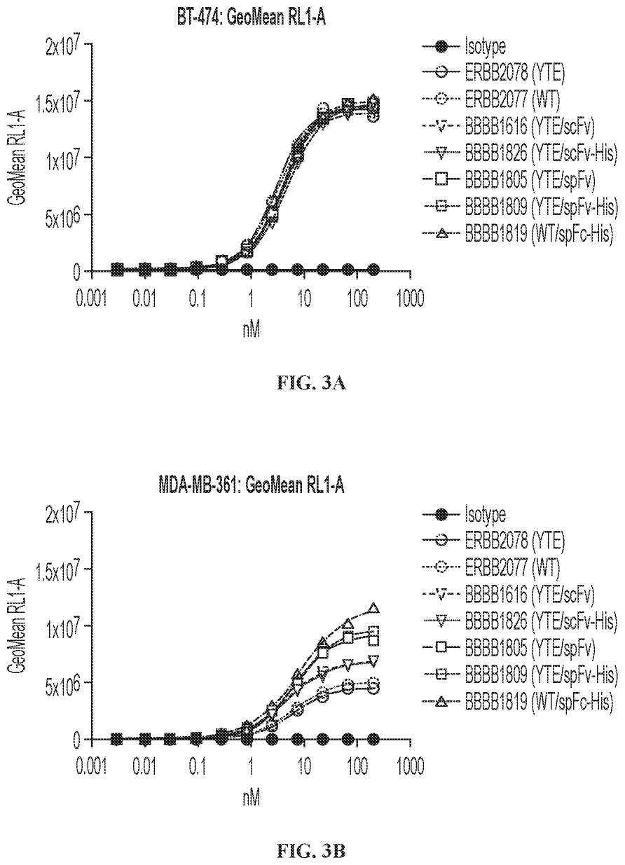

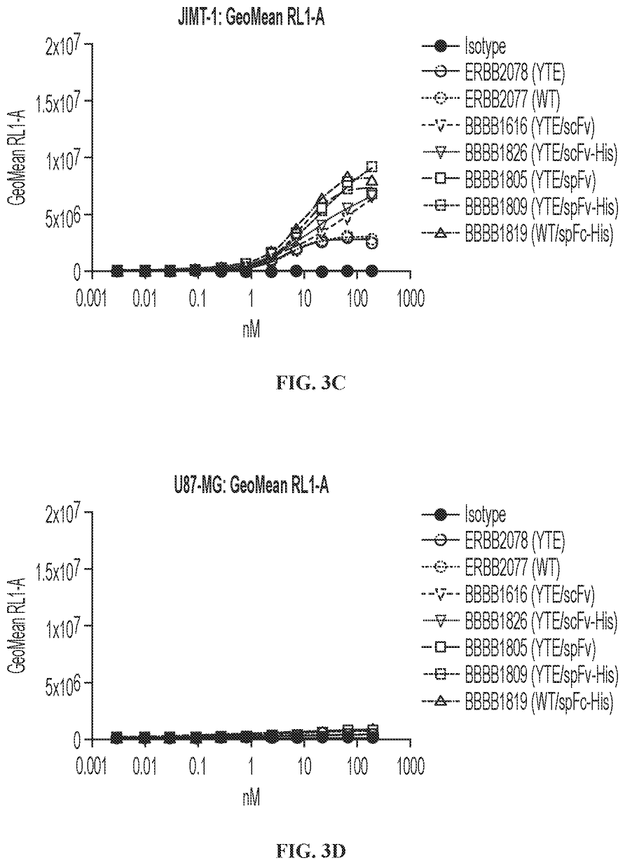

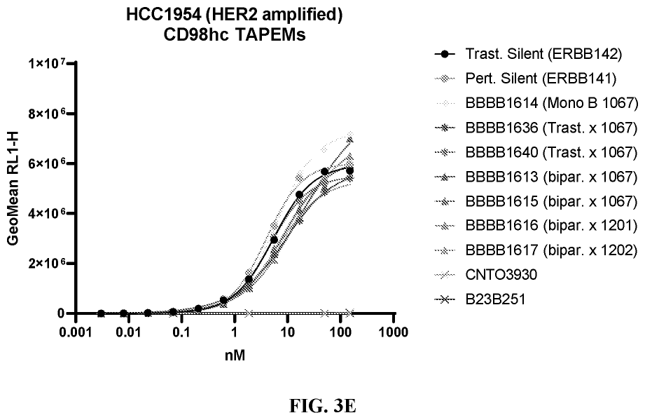

Binding of the TEM mAbs was first tested in HER2 expressing cell lines BT-474, MDA-MB-361, JIMT-1, and U87-MG (FIGS. 3A-D) compared to Isotype control. At very high HER2 expression, binding was mediated by HER2 expression independent of CD98 hc scFv (FIG. 3A). In cell lines expressing high to moderate expression of HER2, the binding of the mAbs was influenced by the CD98 hc with the mAbs containing CD98 spFv showing improved binding over CD98 scFv (FIGS. 3B-C).

TEM mAbs were further test in HCC1954 cell line with amplified HER2 expression. Monovalent (BBBB1614), bivalent (BBBB1636 and BBBB1640), and biparatopic (BBBB1613, BBBB1615, BBBB1616, BBBB1617) were tested along with silent trastuzumab, silent pertuzumab and isotype control (CNTO3930). All antibodies showed a dose dependent binding (FIG. 3E).

### Example 3. TEM Antibodies Promote Antibody-Dependent Cell Cytotoxicity/Phagocytosis

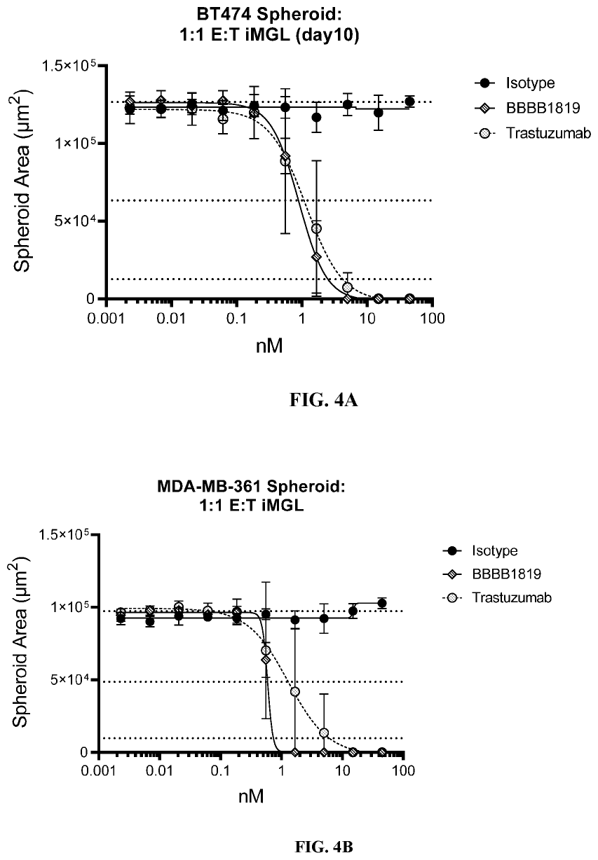

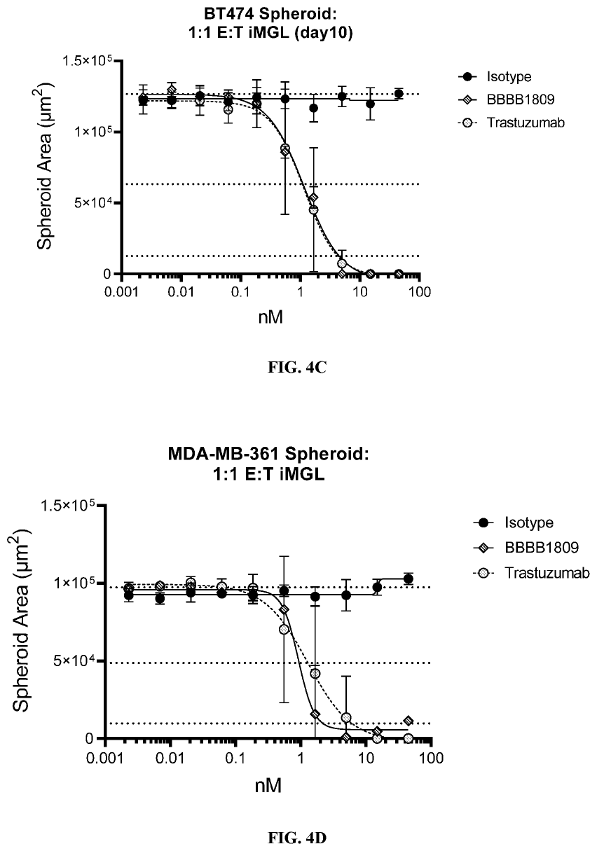

To further test the anti-HER2 TEMs, the HER2-expressing breast cancer cell lines BT474 and MDA-MB-361 were chosen and engineered to stably express red fluorescent protein (RFP) in the nuclei by lentiviral transduction, and used to create tumor spheroids, which are widely believed to be a more physiologic model of tumor growth as compared to 2D cultures. Spheroids were cocultured with human induced pluripotent stem cell (iPSC)-derived microglia cells (iMG) in the presence of anti-HER2 TEMs or controls and fluorescent signal was monitored over time for 10 days (FIGS. 4A-D). For both cell lines, trastuzumab caused a dose-dependent reduction in red fluorescence area at day 10 indicating tumor cell killing. Both biparatopic mAbs (BBBB1809 and BBBB1819) showed a strong dose-dependent decrease in the fluorescent area that was not significantly different than trastuzumab. Taken together, these results demonstrated that anti-HER2 TEM have a strong cytotoxic effect on HER2+ tumor cells that is due to induction of microglial ADCP.

### Example 4. TEM Antibodies Promote ADCC/ADCP of Live Target Cells by PBMCs

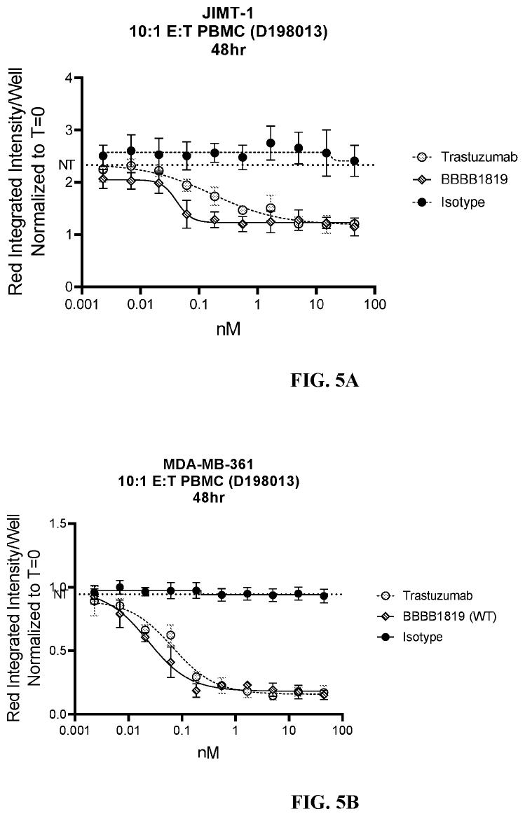

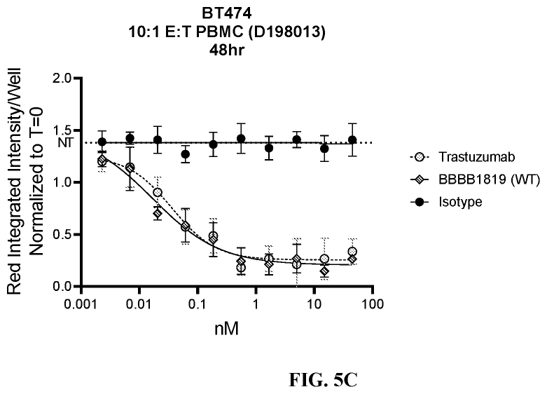

The ability of anti-HER2 TEM molecules to promote clearance of HER2+ tumor cells by human PBMC was next tested. Over the course of 48 hrs in co-culture, a strong cytotoxicity mediated by biparatopic TEMs against JIMT-1, MDA-MB-361, BT474 cell lines was observed (FIG. 5A-C). Potent dose-dependent inhibition of tumor growth by Trastuzumab and biparatopic TEMs was observed. Compared to trastuzumab, the biparatopic HER2-TEM molecules show increased potency toward HER2 moderate cell lines (JIMT-1 and MDA-MB-361), and comparable potency in HER2 high cell line (BT-474).

In total, these data demonstrated that anti HER2 TEM molecules enable microglia and peripheral immune cells to clear tumor cells in an antigen dependent manner.

### Example 5. TEM Results in Enhanced Brain Delivery in Cynomolgus Monkeys

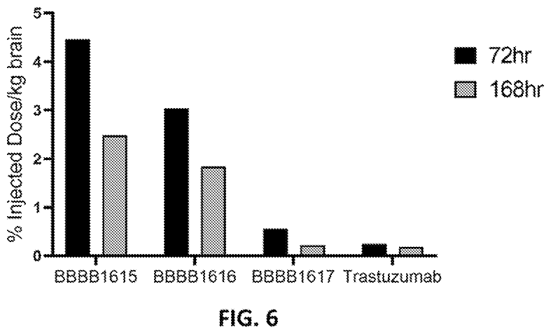

NHPs dosed with the TEM mAbs (BBBB1615, BBBB1616, and BBBB1617) were compared to NHP groups injected with Trastuzumab. Brain mAb concentrations at 72 h and 168 h post IV dosing (10 mg/kg) were measured in eight perfused and capillary-depleted brain regions. Presence of the TEM increased the concentration of mAbs in frontal lobe, hippocampus and temporal lobe compared to trastuzumab, with a wide range of fold increases (FIG. 6). These data demonstrated that anti-Her2 TEMs led to increased mAb uptake in the brain versus trastuzumab.

### Example 6. TEM Anti-Tumor Activity in Mice

The antitumor activity of BBBB1819 as a single agent was evaluated in the BT474 mammary ductal carcinoma cell line xenograft model grown subcutaneous in female immune-compromised NSG (i.e., non-obese diabetic [NOD] severe combined immunodeficiency [scid] gamma, or NOD.Cg Prkdc^{scid} Il2rg^{tm1Wjl}/Szh) mice. Mice were implanted with 1×10⁷ cells and intraperitoneal (IP) dosing was initiated after subcutaneous tumors were established to a target randomization volume of 146 to 155 mm³.

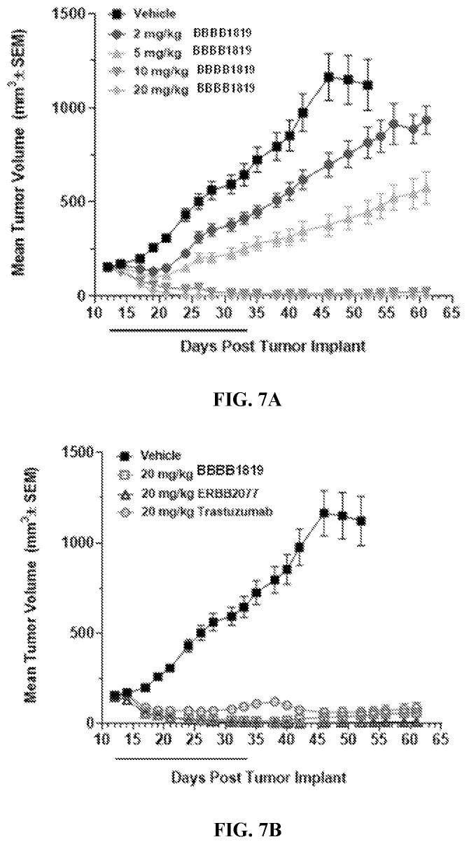

In one portion of the study, the dose-dependent antitumor activity of BBBB1819 was evaluated using a once each 3 days for a total of 8 doses (Q3D×8) schedule, with groups of n=10 animals dosed at 2, 5, 10, or 20 mg/kg BBBB1819, or with phosphate-buffered saline (PBS) control vehicle (FIG. 7A). Δ tumor growth inhibition (TGI) of subcutaneous BT474 xenografts was calculated on Day 52, when at least two thirds of control animals remained on study. Statistically significant ΔTGI was observed with BBBB1819 at 2 mg/kg (31%, p<0.05; Table 5), 5 mg/kg (70%, p<0.05), 10 mg/kg (114%, p<0.05), and 20 mg/kg (116%, p<0.05). Dose-dependent complete responses (defined as no measurable tumor remaining) to BBBB1819 were observed in 0 of 10 animals at 2 mg/kg, 0 of 10 animals at 5 mg/kg, 4 of 10 animals at 10 mg/kg, and 9 of 10 animals at 20 mg/kg by the end of the study.

In another portion of the study, the antitumor activity of BBBB1819 was compared to that of the reference molecules ERBB2077 (the equivalent antibody format to BBBB1819, but lacking the CD98 spFv) and trastuzumab. NSG mice bearing established subcutaneous tumors randomized to groups of n=10 animals were dosed with BBBB1819, ERBB2077, or trastuzumab IP at 20 mg/kg using a once weekly for a total of 4 doses (QW×4) schedule (FIG. 7B). The PBS-treated group from the parallel Q3D×8 cohort was used as a control group. ΔTGI of SC BT474 xenografts was calculated on Day 52, when at least two thirds of control animals remained on study. Statistically significant ΔTGI was observed with BBBB1819 (111%, p<0.05), ERBB2077 (115%, p<0.05), and trastuzumab (109%, p<0.05). BBBB1819 had similar antitumor activity to trastuzumab and ERBB2077. Complete responses were observed in 4 of 10 animals dosed with BBBB1819, 9 of 10 animals dosed with ERBB2077, and 1 of 10 animals dosed with trastuzumab by the end of the study.

TABLE 5
Summary of tumor volumes and % ATGI
from the BT-474 SC tumor study.

| | | Mean | Mean | |
|---|---|---|---|---|
| | | tumor | tumor | |
| | | volume | volume | |
| | | (mm³) | (mm³) | % |
| Group | Schedule | Day 12 | Day 52^{a} | ATGI |
| Vehicle control (PBS) | Q3Dx8 | 155.3 | 1,121.9 | — |
| 2 mg/kg BBBB1819 | Q3Dx8 | 151.3 | 815.4 | 31 |
| 5 mg/kg BBBB1819 | Q3Dx8 | 152.1 | 446.5 | 70 |
| 10 mg/kg BBBB1819 | Q3Dx8 | 152.3 | 15.1 | 114 |
| 20 mg/kg BBBB1819 | Q3Dx8 | 154.4 | 1.5 | 116 |
| 20 mg/kg BBBB1819 | QWx4 | 152.9 | 45.3 | 111 |
| 20 mg/kg ERBB2077 | QWx4 | 145.7 | 5.3 | 115 |
| 20 mg/kg Trastuzumab | QWx4 | 148.6 | 62.5 | 109 |

PBS, phosphate-buffered saline; Q3Dx8, once each 3 days for a total of 8 doses; QWx4, once weekly for a total of 4 doses; SC, subcutaneous; TGI, tumor growth inhibition; TV_{c}, mean tumor volume of a given control group; TV_{c0}, mean initial tumor volume of a given control group; TVₜ, mean tumor volume of treatment group; TVₜ₀, mean initial tumor volume of treatment group.
The pvalue for antitumor activity was p ≤ 0.05 for all treatment groups and thus statistically significant.
% ΔTGI = ([(TV_{c} − TV_{c0}) − (TVₜ − TVₜ₀)] / [TV_{c} − TV_{c0}]) × 100.
^{a}Final day when at least two thirds of animals remained in control group.

Treatment with BBBB1819, ERBB2077, or trastuzumab did not result in significant body weight loss as compared to the PBS-treated control group at any dose in these studies, though it is noted that none of these antibodies are cross-reactive with murine Erbb2 or Slc3a2 (Cd98).

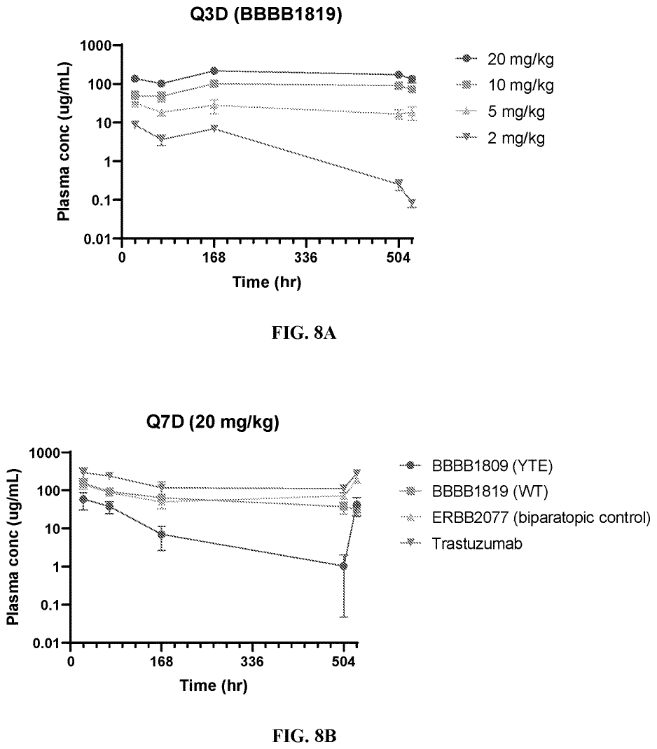

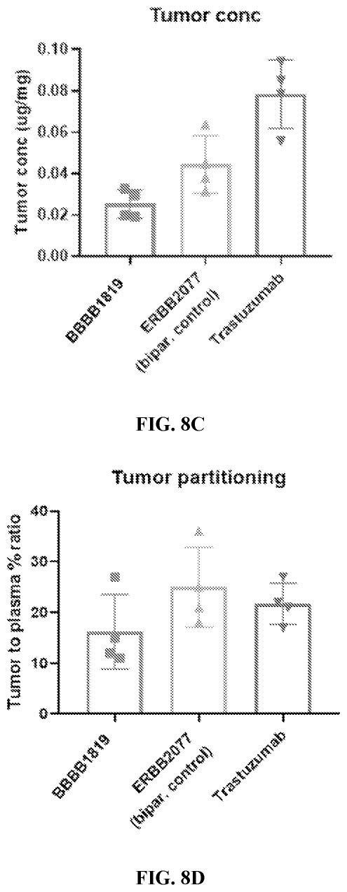

In addition to the efficacy assessments, blood and tumor PK analyses were conducted from satellite groups (n=4 per dose group), with retro-orbital bleeds performed 24 hours post first dose, prior to second dose, 24 hours post third dose, prior to eighth dose (Q3D×8 groups only), and 24 hours post eighth dose (Q3D×8 groups only) (FIGS. 8A and 8B). Additionally, tumor PK analyses were performed from each QW×4 group, with tumor tissues collected 24 hours post first dose (FIGS. 8C and 8D).

The tumor-to-plasma ratio of BBBB1819 was found to be ~15%. Based on mice tumor regression and PK data modeling, the tumor concentration required for 50% tumor cytotoxicity was estimated to be 1.5 μg/mL.

### Example 7. TEM PET/CT Imaging Studies

The in vivo distribution of [Zr89]-DFO*-CD98×HER2 (BBBB1616) IgG antibody in human CD98 knock-in female mice (Biocytogen 110983) and female C57BL6 mice (Jax) mice was evaluated. On the day of the imaging experiment, the tracer precursor was labeled by [Zr89]. Shortly after, the quality control (QC) for compound radiochemical purity and specific activity were measured.

Animal body weight were measured using Mettler Toledo scale. Animals were then anesthetized in anesthesia chamber using Isoflurane with oxygen (3.0%-4.5% for induction and 1.0%-3.0% for maintaining) via Somni AMD-3. After the animal maintaining stable breathing pattern was ensured, the animal was placed on the procedure table for injection with heating pad to maintain the body temperature.

Mice were separated into three groups (A, B, and C). Same group of animals were injected with PET tracer within ~2 min interval. At the designated time point, mice were placed on the 4-mice hotel of Sofie GNEXT PET/CT scanner (Sofie, Culver City, CA, USA) with heating pad to maintain body temperature along with breathing monitoring. Imaging protocol was started with 30 min static PET scan and follow by 1 min standard CT scan acquisition protocol.

### Acquisition and Reconstruction Parameters:

PET/CT study was performed on days 1, 5, and 7 post-tracer injection. The whole body was centered in the axial FOV of the scanner to maximize sensitivity and resolution. CT scan was used for attenuation correction, anatomical images and scatter correction for PET images. PET scan energy window was set between 350 and 650 KeV, 3.438 ns timing window. Emission data were collected in list mode for 30 minutes. PET images reconstructed with iterative 2-dimensional ordered-subsets expectation maximization (OSEM2D) algorithm (4 OSEM2D-iterations, with Fourier rebinning) into single frame. The data were reconstructed into 128× 128 matrix size images.

### Results

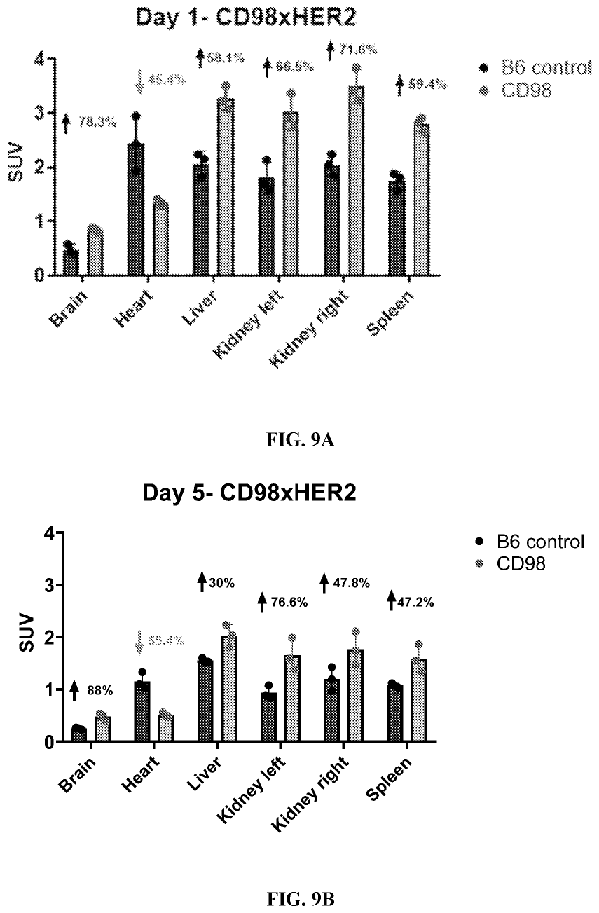

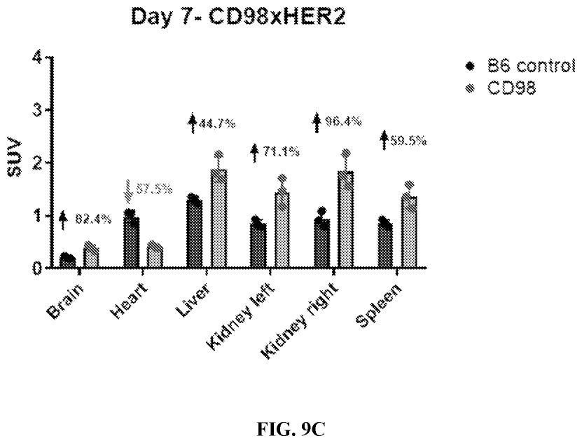

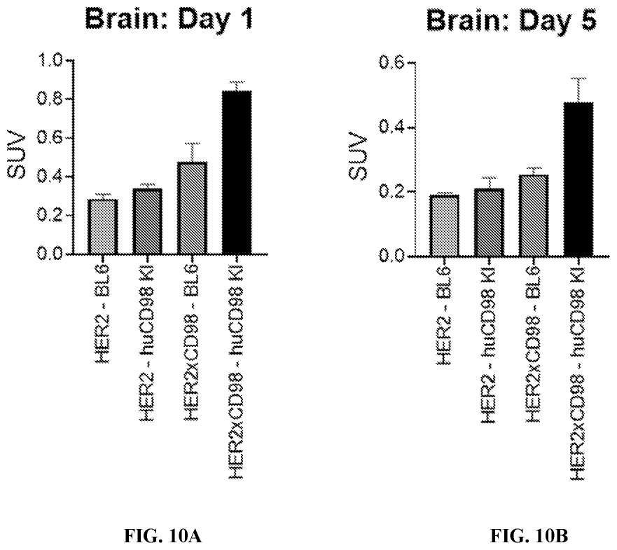

FIGS. 9A-C show distribution of Zr89-DFO*-HER2×CD98 antibody in CD98 knock-in female mice (Biocytogen 110983) and female C57BL6 mice (Jax) mice on 1, 5 and 7 days after injection. The Zr89-DFO*-HER2×CD98 antibody showed higher and homogeneous brain uptake in CD98 knock-in mice (FIGS. 10A-10D). The higher Zr89-DFO*-HER2×CD98 antibody uptake in the brain was seen throughout the experiment while whole body activity was the same in the C57BL6 mice and CD98 knock in mice (FIGS. 11A-C). The Zr89-DFO*-HER2 antibody showed higher peripheral organ uptake (liver, kidney, and spleen) compared to the Zr89-DFO*-HER2×CD98 antibody (Data not shown).

### Example 8. TEM Pharmacokinetic (PK) Analyses in CD98 Knock-In Mice

Additional studies were conducted to determine the pharmacokinetics (PK) of TEM antibodies in human CD98 knock-in (huCD98KI) female mice (Biocytogen 110983). Mice were injected intravenously (i.v.) with 13 mg/kg BBB1819, ERBB2077 or trastuzumab (ERBB128) antibodies. Retro-orbital bleeds were performed 24 hours post-injection. Mice were sacrificed and brain tissue was collected.

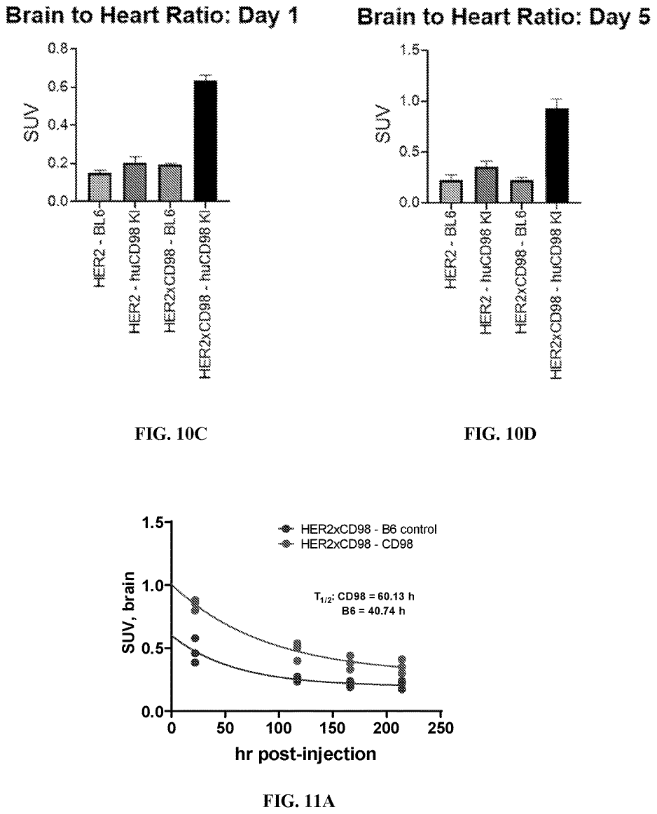

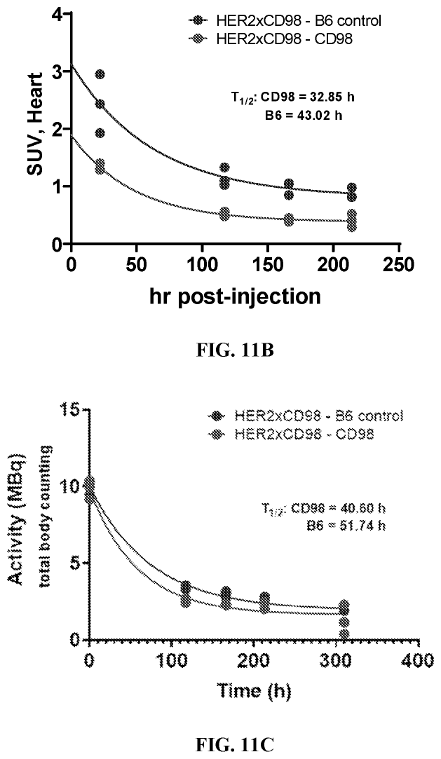

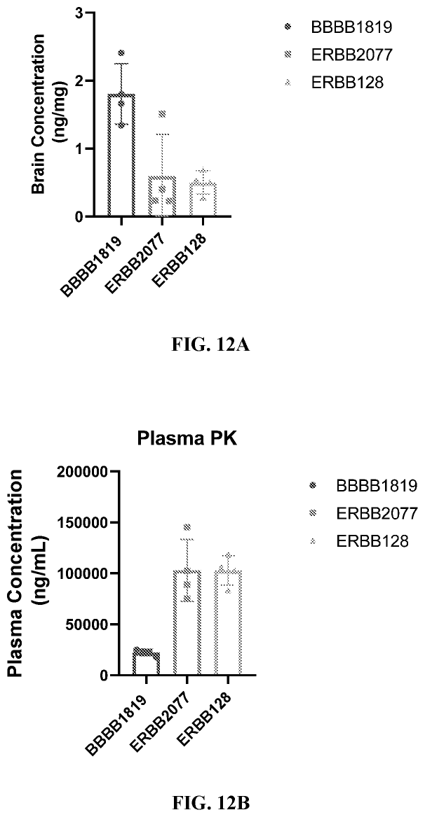

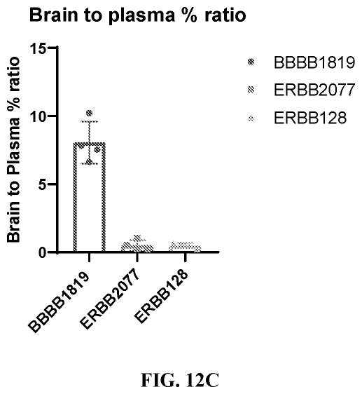

BBBB1819 showed higher concentration in the brain and lower concentration in the plasma of huCD98KI mice compared to ERBB2077 and trastuzumab (FIGS. 12A-C). These data demonstrated that BBBB1819 TEM led to increased mAb uptake in the brain of huCD98KI mice compared to antibodies without the CD98 spFv.

1. A multispecific antibody or antigen binding fragment thereof comprising at least one of a first antigen-binding region and a second antigen-binding region each capable of binding specifically to human epidermal growth factor receptor 2 (HER2), and a third antigen-binding region capable of binding specifically to CD98, wherein:
(1) the first antigen-binding region comprises:
a first heavy chain variable region (VH1) comprising a heavy chain complementarity determining region 1 (HCDR1), a HCDR2, and a HCDR3 comprising the amino acid sequences of SEQ ID NOs: 5, 6 and 7, respectively;
and a first light chain variable region (VL1) comprising a light chain complementarity determining region 1 (LCDR1), a LCDR2, and a LCDR3 comprising the amino acid sequences of SEQ ID NOs: 8, 9 and 10, respectively;
(2) the second antigen-binding region comprises:
second heavy chain variable region (VH2) comprising a heavy chain complementarity determining region 1 (HCDR1), a HCDR2, and a HCDR3 comprising the amino acid sequences of SEQ ID NOs: 13, 14 and 15, respectively; and a second light chain variable region (VL2) comprising a light chain complementarity determining region 1 (LCDR1), a LCDR2, and a LCDR3 comprising the amino acid sequences of SEQ ID NOs: 16, 17 and 18, respectively; and
(3) the third antigen-binding region comprises a first single chain variable fragment (scFv1) having: a third heavy chain variable region (VH3) comprising a heavy chain complementarity determining region 1 (HCDR1), a HCDR2, and a HCDR3, and a third light chain variable region (VL3) comprising a light chain complementarity determining region 1 (LCDR1), a LCDR2, and a LCDR3, wherein the HCDR1, HCDR2, HCDR3, LCDR1, LCDR2 and LCDR3 have any of the amino acid sequences of Table 2.

2. The multispecific antibody or antigen binding fragment thereof of claim 2, wherein:
(1) the VH1 an amino acid sequence identical to the VH of the HC sequence identified as SEQ ID NO: 1; and the VL1 comprises an amino acid sequence identical to the VL of the LC sequence identified as SEQ ID NO: 2;
(2) the VH2 an amino acid sequence identical to the VH of the HC sequence identified as SEQ ID NO: 11; and the VL2 comprises an amino acid sequence identical to the VL of the LC sequence identified as SEQ ID NO: 12; and
(3) the VH3 and the VL3 comprise amino acid sequences are identical to the respective VH and VL of a scFv identified as:
(i) SEQ ID NO: 19;
(ii) SEQ ID NO: 26;
(iii) SEQ ID NO: 33;
(iv) SEQ ID NO: 34;
(v) SEQ ID NO: 35;
(vi) SEQ ID NO: 36;
(vii) SEQ ID NO: 43;
(viii) SEQ ID NO: 49;
(ix) SEQ ID NO: 55;
(x) SEQ ID NO: 59;
(xi) SEQ ID NO: 66;
(xii) SEQ ID NO: 71;
(xiii) SEQ ID NO: 78;
(xiv) SEQ ID NO: 85;
(xv) SEQ ID NO: 87;
(xvi) SEQ ID NO: 88;
(xvii) SEQ ID NO: 89;
(xviii) SEQ ID NO: 90;
(xix) SEQ ID NO: 92;
(xx) SEQ ID NO: 93;
(xxi) SEQ ID NO: 94.

3. The multispecific antibody or antigen binding fragment thereof of claim 1, comprising:
(a) a first heavy chain (HC1) comprising the VH1, a first heavy chain constant region comprising a first Fc region (Fc1), and the scFv1, and
(b) a first light chain (LC1) comprising the VL1 and a light chain constant region.

4. The multispecific antibody or antigen binding fragment thereof of claim 3, further comprising:
(a) a second heavy chain (HC2) comprising the VH1 and a first heavy chain constant region comprising a second Fc region (Fc2), and
(b) a second light chain (LC2) comprising the VL1 and a light chain constant region.

5. The multispecific antibody or antigen binding fragment thereof of claim 3, further comprising a second Fc region (Fc2).

6. The multispecific antibody or antigen binding fragment thereof of claim 1, comprising:
(a) a first heavy chain (HC1) comprising the VH1, and a first heavy chain constant region comprising a first Fc region (Fc1),
(b) a first light chain (LC1) comprising the VL1 and a light chain constant region; and
(c) a second heavy chain (HC2) comprising the scFv1, and a second heavy chain constant region comprising a second Fc region (Fc2).

7. The multispecific antibody or antigen binding fragment thereof of claim 1, comprising:
(a) a first heavy chain (HC1) comprising the VH2, a first heavy chain constant region comprising a first Fc region (Fc1), and the scFv1, and
(b) a first light chain (LC1) comprising the VL2 and a light chain constant region.

8. The multispecific antibody or antigen binding fragment thereof of claim 7, further comprising:
(a) a second heavy chain (HC2) comprising the VH2 and a first heavy chain constant region comprising a second Fc region (Fc2), and
(b) a second light chain (LC2) comprising the VL2 and a light chain constant region.

9. (canceled)

10. The multispecific antibody or antigen binding fragment thereof of claim 1, comprising:
(a) a first heavy chain (HC1) comprising the VH2, and a first heavy chain constant region comprising a first Fc region (Fc1),
(b) a first light chain (LC1) comprising the VL2 and a light chain constant region; and
(c) a second heavy chain (HC2) comprising the scFv1, and a second heavy chain constant region comprising a second Fc region (Fc2).

11. The multispecific antibody or antigen binding fragment thereof of claim 1, comprising the first antigen binding region, the second antigen binding region and the third antigen binding region.

12. The multispecific antibody or antigen binding fragment thereof of claim 11, comprising:
(a) a first heavy chain (HC1) comprising the VH1, a first heavy chain constant region comprising a first Fc region (Fc1), and the scFv1,
(b) a first light chain (LC1) comprising the VL1 and a light chain constant region; and
(c) a second heavy chain (HC2) comprising a second single chain variable fragment (scFv2) and a first heavy chain constant region comprising a second Fc region (Fc2), wherein the scFv2 comprises the VH2 and VL2.

13. The multispecific antibody or antigen binding fragment thereof of claim 11, comprising:
(a) a first heavy chain (HC1) comprising the VH2, a first heavy chain constant region comprising a first Fc region (Fc1), and the scFv1,
(b) a first light chain (LC1) comprising the VL2 and a light chain constant region; and
(c) a second heavy chain (HC2) comprising a second single chain variable fragment (scFv2) and a first heavy chain constant region comprising a second Fc region (Fc2), wherein the scFv2 comprises the VH1 and VL1.

14. The multispecific antibody or antigen binding fragment thereof of claim 12, wherein the scFv1 and/or scFv2 comprises at least one of (a) a first disulfide bond between a structurally conserved surface exposed VH cysteine (Cys) and a first L Cys; and b) a second disulfide bond between a structurally conserved surface exposed VL Cys and a second L Cys.

15. (canceled)

16. The multispecific antibody or antigen binding fragment thereof of claim 13, wherein the scFv2 comprises an amino acid sequence having an amino acid sequence of SEQ ID NO: 3 or 4.

17. The multispecific antibody or antigen binding fragment thereof of claim 1, wherein the scFv1 comprises an amino acid sequence selected from the group consisting of: SEQ ID NO: SEQ ID NO: 19, SEQ ID NO: 26, SEQ ID NO: 33, SEQ ID NO: 34, SEQ ID NO: 35, SEQ ID NO: 36, SEQ ID NO: 43, SEQ ID NO: 49, SEQ ID NO: 55, SEQ ID NO: 59, SEQ ID NO: 66, SEQ ID NO: 71, SEQ ID NO: 78, SEQ ID NO: 85, SEQ ID NO: 87, SEQ ID NO: 88, SEQ ID NO: 89, SEQ ID NO: 90, SEQ ID NO: 92, SEQ ID NO: 93, SEQ ID NO: 94.

18. The multispecific antibody or antigen binding fragment thereof of claim 6, wherein each of the Fc1 and Fc2 comprises one or more heterodimeric mutations, or one or more knob and hole mutations.

19. The multispecific antibody or antigen binding fragment thereof of claim 18, wherein the heterodimeric mutations comprise amino acid modifications at positions T350, L351, F405, and Y407 in one of Fc1 and Fc2, and amino acid modifications at positions T350, T366, K392 and T394 in the other one of Fc1 and Fc2, wherein the amino acid modification at position T350 is T350V, T350I, T350L or T350M; the amino acid modification at position L351 is L351Y; the amino acid modification at position F405 is F405A, F405V, F405T or F405S; the amino acid modification at position Y407 is Y407V, Y407A or Y407I; the amino acid modification at position T366 is T366L, T366I, T366V or T366M, the amino acid modification at position K392 is K392F, K392L or K392M, and the amino acid modification at position T394 is T394W, and wherein the numbering of amino acid residues is according to the EU index as set forth in Kabat.

20.-32. (canceled)

33. A method of treating or detecting a disorder, preferably a cancer, in a subject in need thereof, comprising administering to the subject the multispecific antibody or antigen-binding fragment of claim 1, or the pharmaceutical composition of claim 32.

34. The method of claim 33, wherein the disease or disorder is a HER2 associated disease or disorder.

35. The method of claim 34, wherein the disease or disorder is brain metastasis.
